(19) United States
(12) Patent Application Publication
(10) Pub. No.: US 2023/0053729 A1
(43) Pub. Date: Feb. 23, 2023

(54) A RECOMBINANT FILAMENTOUS FUNGUS FOR PRODUCING ETHANOL AND ITS CONSTRUCTION AND APPLICATION

(71) Applicant: TIANJIN INSTITUTE OF INDUSTRIAL BIOTECHNOLOGY, CHINESE ACADEMY OF SCIENCES, Tianjin (CN)

(72) Inventors: Chaoguang TIAN, Tianjin (CN); Yongli ZHANG, Tianjin (CN); Jingen LI, Tianjin (CN); Tao SUN, Tianjin (CN); Qian LIU, Tianjin (CN); Wenliang SUN, Tianjin (CN)

(21) Appl. No.: 17/786,997

(22) PCT Filed: Dec. 9, 2020
(86) PCT No.: PCT/CN2020/134922

§ 371 Date: Jun. 17, 2022

(30) Foreign Application Priority Data
Dec. 24, 2019 (CN) 201911349044.4
Aug. 27, 2020 (CN) 202010874879.8
Nov. 17, 2020 (CN) 202011283377.4

(51) Int. Cl. C12N 15/80; C12N 9/02; C12N 1/14

(52) U.S. Cl. CPC C12N 15/80; C12N 9/0004; C12N 1/14; C12Y 101/01001

(57) ABSTRACT

The invention discloses a construction method of genetic engineering fungi of filamentous fungi. Through the genetic engineering method, the filamentous fungi overexpress the positive regulation genes of ethanol synthesis, and/or down regulate the negative regulation genes of endogenous ethanol synthesis to obtain genetic engineering strains. Or overexpression of acetaldehyde dehydrogenase and ethanol dehydrogenase containing mitochondrial localization signal sequence, or overexpression of pyruvate decarboxylase and ethanol dehydrogenase containing mitochondrial localization signal sequence, or overexpression of acetaldehyde dehydrogenase, ethanol dehydrogenase and pyruvate decarboxylase containing mitochondrial localization signal sequence in filamentous fungal cells. Compared with the original strain, the ethanol synthesis ability of the obtained genetically engineered strains are improved.

### TECHNICAL FIELD

The invention belongs to the field of genetic engineering and biotechnology. Specifically, the invention relates to a method for constructing a recombinant engineering strain for producing ethanol and the obtained recombinant engineering strain. The invention also relates to a method for producing ethanol by the recombinant engineering fungus.

### BACKGROUND

With the rapid development of the world economy, human beings demand for energy is increasing. On the one hand, the reserves of fossil energy such as petroleum are limited, and a lot of pollution will be produced in the combustion process. Energy and environmental issues have become a major challenge for human development. Transportation industry is the main sector of energy consumption and an important way of greenhouse gas emission, so there is an urgent need to develop clean and renewable alternative fuels. Among them, fuel ethanol has the characteristics of rich raw materials, clean and renewable and good integration with the existing fuel system, and has become the most important alternative energy at present (Zabed, H., Sahu, J. N., Suely, A., Boyce, A. N. and Faruq, G. (2017) Bioethanol production from renewable sources: current perspectives and technological progress. Renewable and Sustainable Energy Reviews, 71, 475-501). In the production of fuel ethanol, the raw materials used in the production of the first generation of fuel ethanol are mainly sugarcane and corn. With the rapid growth of fuel ethanol consumption, the disadvantages of the first generation of fuel ethanol “competing with people for food and land” are becoming increasingly prominent. The second generation fuel ethanol with non grain lignocellulose as raw materials can solve this problem well, and is in line with the strategic policy of sustainable development of China's energy economy. China is rich in lignocellulose resources, and the total output of crop straw produced each year is about 900 million tons (Tian F, Li F, Yuan J, Xu K, Wang K, Wang C, Shu M, Li Y, Tong Y, Cui Z. Current situation of cellulose ethanol industry and technical difficulties in key processes Modern Chemical Industry, 2019,48 (09): 2051-2056). Most of them are treated by incineration or rough return to the field, which not only causes a huge waste of resources, but also causes serious environmental pollution. Making full use of this huge amount of biomass resources to produce fuel ethanol will provide an important guarantee for China's energy security, optimize the energy structure, improve the ecological environment and promote the economic development of rural areas.

At present, the main fermentation microorganisms used in the production of fuel ethanol are S. cerevisiae and Zymomonas mobilis (Sarris, D. and Papanikolaou, S. (2016) Biotechnological production of ethanol: Biochemistry, processes and technologies. Engineering in Life Sciences, 16, 307-329), which showed excellent production performance in the first generation of grain ethanol production. However, in the production of the second generation fuel ethanol, S. cerevisiae and Zymomonas mobilis can not meet the production requirements due to the lack of cellulase secretion and pentose metabolism (especially xylose and arabinose). The main components of lignocellulose are lignin, cellulose and hemicellulose. Cellulose is D-glucose β-1,4 glycosidic bond linked chain polymer compound; hemicellulose is a heterogeneous polymer composed of several different types of monosaccharides, including pentoses such as xylose, arabinose and hexoses such as mannose and galactose. The saccharification products of lignocellulose are mainly about 70% cellobiose and glucose and about 30% xylose (Carroll A, Somerville C. Cellulosic biofuels. Annu Rev Plant Biol. 2009; 60:165-82). In order to improve the fermentation efficiency of xylose, scientists have made many efforts. However, the productivity and yield of ethanol produced by xylose fermentation of S. cerevisiae and Zymomonas mobilis engineering strains are still lower compared with that of glucose fermentation (Sharma N K, Behera S, Arora R, Kumar S, Sani R K. Xylose transport in yeast for lignocellulosic ethanol production: Current status. J Biosci Bioeng. 2018; 125 (3): 259-67). In addition, due to the existence of glucose inhibition effect, natural S. cerevisiae can not use xylose in the presence of glucose, which also hinders the effective utilization of mixed sugar in cellulose hydrolysates.

Cellulase from fungi can hydrolyze cellulose to produce cellobiose, while glucosidase can further hydrolyze cellobiose to glucose. Therefore, the production of biofuels from biomass requires microorganisms that can effectively use cellobiose and glucose. Because glucose inhibits the activity of cellulase and the utilization of other sugars in the fermentation process, the direct utilization of cellobiose by microorganisms is a better method. The degradation of cellobiose to glucose occurs in the body, so it can effectively reduce the inhibitory effect of carbon metabolism in the process of mixed sugar fermentation. However, natural S. cerevisiae cannot use disaccharide fermentation. Only the introduction of exogenous cellobiose utilization pathway can make use of cellobiose, which mainly includes cellobiose transporters from Neurospora for cellobiose induction (Kim H, Lee W H, Galazka J M, Cate J H, Jin YS. Analysis of cellodextrin transporters from M. thermophila in S. cerevisiae for cellobiose fermentation. Appl Microbiol Biotechnol. 2014; 98(3):1087-94) and hydrolysis system or cellobiose phosphorylation system replaced by marine bacterium Saccharophagus degradans (Sadie C J, Rose S H, den Haan R, van Zyl W H. Co-expression of a cellobiose phosphorylase and lactose permease enables intracellular cellobiose utilisation by S. cerevisiae. Appl Microbiol Biotechnol. 2011; 90(4):1373-80). Although S. cerevisiae has been further modified, its ability to use cellobiose is still greatly limited.

Cellulose degrading thermophilic fungi are excellent producers of high temperature resistant cellulases. They naturally have the ability to secrete a large number of biomass degrading enzymes, and can make full use of a variety of sugars such as cellobiose, glucose and xylose produced by degradation (Karnaouri A, Topakas E, Antonopoulou I, Christakopoulos P. Genomic insights into the fungal lignocellulolytic system of M. thermophila. Front Microbiol. 2014; 5:281). At the same time, the optimum growth temperature of these microorganisms is high, which can save the amount of cooling water in the fermentation process and reduce the production cost. It has been proved that it can be used for large-scale industrial fermentation (Visser H, Joosten V, Punt P J, Gusakov A V, Olson P T, Joosten R, Bartels J, Visser J. Development of a mature fungal technology and production platform for industrial enzymes based on a M. thermophila isolate, previously known as Chrysosporium lucknowense Cl. Ind Biotechnol. 2011; 7:214-23). However, the ethanol production of wild-type filamentous fungi (including M. thermophila) is low, so they can not be directly used in the actual production of ethanol. Therefore, genetic transformation of cellulose degrading fungi to realize the direct fermentation of biomass raw materials to ethanol is an urgent problem to be overcome in the production of second-generation fuel ethanol, and it also has great application potential.

In order to realize the one-step fermentation of filamentous fungi from cellulose to ethanol, the characteristics of low ethanol yield and slow metabolic rate of filamentous fungi must be overcome. The level of ethanol production and the speed of metabolic rate largely depend on the strength of glycolysis pathway, and the most important regulatory enzyme in glycolysis pathway is phosphofructokinase. Therefore, regulating the activity of phosphofructokinase is one of the keys to accelerate the glycolysis rate of filamentous fungi and improve ethanol production. At the same time, in order to realize the efficient production of cellulosic ethanol by filamentous fungi, it is necessary to improve the ethanol synthesis capacity of filamentous fungi. However, unlike Saccharomyces cerevisiae, which can anaerobic ferment ethanol, most filamentous fungi are aerobic fungi, and a large amount of carbon sources will enter mitochondria and eventually convert into CO₂ through tricarboxylic acid cycle and respiration, which makes it difficult to greatly improve the yield of ethanol. Therefore, if we want to use filamentous fungi to synthesize ethanol efficiently with cellulose as substrates in one step, we must overcome the above bottleneck.

### Invention Contents

At present, the fermentation production of ethanol mainly takes grain crops such as corn and sugarcane and non grain crops such as cassava as raw materials. Cellulose degrading filamentous fungi represented by M. thermophila can directly use lignocellulose to produce ethanol, which greatly reduces the pretreatment cost and simplifies the production process. However, the ethanol production of wild-type filamentous fungi (including M. thermophila) is low, which can not meet the production demand. Moreover, the genetic operation of filamentous fungi is complex, inefficient and long cycle, which also restricts the process of using filamentous fungi for ethanol production through genetic operation. In recent years, the inventors have successively established the genetic operating system of Rhizopus thermophila (Xu, J., Li, J., Lin, L., Liu, Q., Sun, W., Huang, B. and Tian, C. (2015) Development of genetic tools for M. thermophila. BMC Biotechnol, 15, 35) and gene editing system (Liu, Q., Gao, R., Li, J., Lin, L., Zhao, J., Sun, W. and Tian, C. (2017) Development of a genome-editing CRISPR/Cas9 system in thermophilic fungal M. species and its application to hyper-cellulase production strain engineering. Biotechnol Biofuels, 10, 1), greatly accelerated the genetic transformation process of M. thermophila. On the basis of the above, the inventors also systematically studied the mechanism of ethanol production by filamentous fungi (especially M. thermophila), and significantly improved the efficiency of ethanol production by M. thermophila (the carbon sources used include glucose, xylose, fructose, cellobiose, cellulose and xylan), providing a new strategy for the production of second-generation fuel ethanol. The strategies of genetic transformation include: overexpression of glucose and cellobiose transporter to improve the absorption rate of cellulose degradation products by M. thermophila; overexpression of key genes in ethanol synthesis pathway to improve the efficiency of ethanol synthesis; knock out the main branching pathways; increase the level of cytoplasmic NADH; accelerate the rate of glycolysis; knock out the endogenous ethanol consumption pathway; and overexpression of ethanol synthesis gene containing mitochondrial localization signal sequence for ethanol synthesis.

The invention first provides a construction method of filamentous fungi recombinant engineering fungi, which is characterized in that the key genes of ethanol synthesis pathway are overexpressed in the filamentous fungi, and/or the expression of endogenous ethanol synthesis competitive pathway genes is down regulated, and/or the ethanol synthesis gene containing mitochondrial localization signal sequence is overexpressed in the filamentous fungi for ethanol synthesis through genetic engineering, compared with the original strain, the ethanol synthesis ability of the genetically engineered strain is improved.

Preferably, the filamentous fungi are cellulose degrading filamentous fungi. Specifically, the filamentous fungi are selected from Neurospora, Aspergillus, Trichoderma, Penicillium, Myceliophthora, Sporotrichum, Fusarium, Rhizopus, Mucor and Paecilomyces.

In a more preferred manner, the Myceliophthora is selected from the group consisting of M thermophila and M. heterothalica.

On the other hand, the positive regulation gene of ethanol synthesis is selected from the genes that strengthen the pathway of ethanol synthesis, improve the ability of sugar transport, accelerate the rate of glycolysis and improve the level of cytoplasmic NADH; the negative regulation gene of ethanol synthesis is selected from the genes in the branch pathway of ethanol synthesis, energy metabolism, the shuttle of cytoplasmic reducing force to mitochondria and the endogenous ethanol metabolism pathway.

In some examples, the shuttle of cytoplasmic reducing force to mitochondria is reduced or blocked, and/or the energy metabolism pathway is modified, and/or the ethanol synthesis pathway is strengthened, and/or the sugar molecule transport is strengthened, and/or the ethanol synthesis by-product pathway is weakened, and/or the glycolysis rate is accelerated.

In another aspect, the overexpression of the positive regulation gene of ethanol synthesis is realized by introducing exogenous and/or endogenous positive regulation genes of ethanol synthesis, wherein the positive regulation gene of ethanol synthesis is selected from one of ethanol dehydrogenase, glucose transporter, cellobiose transporter, or pyruvate decarboxylase, or a variety of combinations thereof. More specifically, overexpression of exogenous ethanol dehydrogenase to improve ethanol production capacity, or overexpression of exogenous glucose transporter to improve ethanol production capacity, or overexpression of exogenous cellobiose transporter to improve ethanol production capacity, and overexpression of exogenous pyruvate decarboxylase to improve ethanol production capacity.

Preferably, the introduction is to transfer the expression vector carrying the positive regulation genes of exogenous or endogenous ethanol synthesis into the host cell, and the preferred promoters are tef, gpdA, trpC, cbhl and glaA promoters.

More preferably, the introduced positive regulatory gene of exogenous ethanol synthesis comes from yeast, preferably from S. cerevisiae and Zymomonas mobilis.

In some examples, the ethanol dehydrogenase coding gene Scadh1 and/or pyruvate decarboxylase gene Scpdc1 and/or glucose transporter coding gene glt-1 and/or cellobiose transporter coding gene cdt-1/cdt-2 are overexpressed in the filamentous fungi.

In some specific examples, the overexpression of the positive regulation gene of ethanol synthesis in the filamentous fungus is selected from exogenous ethanol dehydrogenase, and the preferred ethanol dehydrogenase is ethanol dehydrogenase Scadh1, and/or ZmADH1 from Zymomonas mobilis, the preferred pyruvate decarboxylases are ScPDC1 and ScPDC5 from Saccharomyces cerevisiae, and/or ZmPDC from Zymomonas mobilis.

In another example, the down-regulated gene expression is to inactivate the related genes or reduce the expression or activity by gene knockout or small RNA interfering or changing the promoter or gene mutation. Preferably, the gene editing is a genome editing method based on CRISPR/Cas9.

In some specific examples, the endogenous ethanol synthesis negative regulation gene is selected from the genes in the branch pathway of ethanol synthesis: ldh1, ldh2, mpd; the genes in the shuttle pathway of cytoplasmic reducing power to mitochondria: mdh, gpd, nde1, nde2; the genes in the electron transport chain (respiratory chain): cox; and the genes of ethanol metabolism: Mtadh, Mtaldh. More specifically, it is selected from one of lactate dehydrogenase, mannitol-1-phosphate dehydrogenase, cytoplasmic malate dehydrogenase, glycerol-3-phosphate dehydrogenase, cytochrome C oxidase, outer NADH dehydrogenase, ethanol dehydrogenase, acetaldehyde dehydrogenase, or a variety of combinations thereof. More specifically, the expression of lactate dehydrogenase gene, a negative regulator of endogenous ethanol synthesis, was decreased or lost in the strain, so as to improve the ability of ethanol production; either reduce or lose the expression of endogenous mannitol-1-phosphate dehydrogenase gene to improve the ability of ethanol production, or reduce or lose the expression of endogenous cytoplasmic malate dehydrogenase gene to improve the ability of ethanol production, or reduce or lose the expression of endogenous glycerol-3-phosphate dehydrogenase gene to improve the ability of ethanol production, or reduce or lose the expression of endogenous ethanol dehydrogenase (Mycth_55576) gene, or reduce or lose the expression of one or more combination genes in endogenous acetaldehyde dehydrogenase, or reduce the expression of endogenous cytochrome C oxidase in the strain.

In some examples, the endogenous ethanol synthesis negative regulatory gene is selected from the lactate dehydrogenase gene and/or 1-phosphate mannitol dehydrogenase.

In other examples, the endogenous ethanol synthesis negative regulatory gene is a cytochrome C oxidase coding gene and/or an external NADH dehydrogenase gene.

In some examples, the filamentous fungi overexpress the ethanol dehydrogenase of Saccharomyces cerevisiae, the cellobiose transporter coding gene cdt-1/cdt-2 from Neurospora crassa, and down regulate the expression of endogenous lactate dehydrogenase genes ldh-1 and/or ldh-2.

In some examples, the ethanol dehydrogenase of Saccharomyces cerevisiae is overexpressed in the filamentous fungus and the expression of the outer NADH dehydrogenase gene nde is down regulated, wherein one or both of the outer NADH dehydrogenase genes nde1 and nde2 are down regulated.

In some examples, ethanol dehydrogenase of Saccharomyces cerevisiae is overexpressed in the filamentous fungus and the expression of cytochrome C oxidase gene is down regulated.

In some examples, ethanol dehydrogenase of Saccharomyces cerevisiae is overexpressed in the filamentous fungus, the expression of cytoplasmic malate dehydrogenase gene mdh is down regulated, and the expression of glycerol-3-phosphate dehydrogenase gene gpd is optionally further down regulated.

In some specific examples, the ethanol dehydrogenase gene Scadh1 and pyruvate decarboxylase gene Scpdc1 of Saccharomyces cerevisiae are overexpressed in the filamentous fungus, optionally further overexpressing the glucose transporter coding gene glt-1, and further regulating the expression of lactate dehydrogenase gene and mannitol 1-phosphate dehydrogenase gene. Or further down regulate the cytoplasmic malate dehydrogenase gene mdh, and/or down regulate the ethanol dehydrogenase gene Mtadh and/or acetaldehyde dehydrogenase gene Mtaldh.

In some specific examples, the ethanol dehydrogenase gene Scadh1 and pyruvate decarboxylase gene Scpdc1 of Saccharomyces cerevisiae are overexpressed in the filamentous fungi, optionally further overexpressing the glucose transporter coding gene glt-1, and further regulating the expression of endogenous lactate dehydrogenase gene ldh1/ldh2 and 1-phosphate mannitol dehydrogenase gene mpd.

In some examples, the Saccharomyces cerevisiae ethanol dehydrogenase gene Scadh1 and the pyruvate decarboxylase gene Scpdc1 are overexpressed in the filamentous fungus.

In another examples, the endogenous phosphofructokinase 2 gene is knocked out in the filamentous fungus, and the mutant phosphofructokinase 2 gene is preferably further expressed, wherein the mutant phosphofructokinase 2 refers to the retention of kinase activity and the loss or reduction of phosphokinase activity after mutation; the phosphofructose kinase 2 refers to the gene encoding the synthesis and degradation of fructose-2,6-diphosphate or its homologous gene; the mutated phosphofructose kinase 2 refers to gene ID (herein, gene ID refers to NCBI https://www.ncbi.nlm.nih.gov/gene number; the same below): 11510101 is a mutant corresponding to one or two or three sites in H233, E306 and H371 in the amino acid sequence to reduce or lose phosphatase activity. In this examples, in order to promote the glucose metabolism rate and improve the ethanol yield, the invention mutates the phosphofructokinase 2 gene to obtain a mutant gene that can promote the glucose metabolism rate and improve the ethanol yield. At the same time, in order to further verify whether knockout of phosphofructokinase 2 and overexpression of mutant phosphofructokinase 2 can also promote glucose metabolism rate and improve ethanol production, the invention has carried out experimental verification on phosphofructokinase 2 knockout strain and strain overexpressing the gene mutant.

In other examples, overexpression of ethanol synthesis gene containing mitochondrial localization signal sequence in the filamentous fungus is used to synthesize ethanol by overexpression of acetaldehyde dehydrogenase and ethanol dehydrogenase genes containing mitochondrial localization signal sequence, or overexpression of pyruvate decarboxylase and ethanol dehydrogenase genes containing mitochondrial localization signal sequence, or overexpression of acetaldehyde dehydrogenase, ethanol dehydrogenase and pyruvate decarboxylase genes containing mitochondrial localization signal sequence. Preferably, the pyruvate decarboxylase and ethanol dehydrogenase are derived from Saccharomyces cerevisiae or Zymomonas mobil's; the acetaldehyde dehydrogenase comes from Thermoanaerobacterium saccharolyticum. In a preferred examples, the mitochondrial localization signal sequence refers to the N-terminal of the amino acid sequence used to guide the transmembrane transfer of protein to mitochondria. Preferably, the localization signal sequence derived from the mitochondrial matrix protein of M. thermophila is selected. In these examples, the invention hopes to use the abundant pyruvate, acetyl CoA and NADH in mitochondria to directly synthesize ethanol in mitochondria. The verification shows that the above strategy overcomes the defect that filamentous fungi make a large number of carbon sources enter mitochondria due to aerobic respiration, and finally convert into carbon dioxide after passing through the tricarboxylic acid cycle, which makes it difficult to improve ethanol production, and realizes the effective improvement of ethanol production.

The invention also provides a genetically engineered fungus obtained by the construction method as described above.

In the examples of the invention, compared with the original strain, the ethanol production capacity is enhanced or increased by at least 10%; preferably at least 20%, 30%, 40%, 50%.

The invention further provides the use of the recombinant fungus with improved ethanol production capacity obtained by the above construction method in the production of ethanol.

Among them, it uses monosaccharide or/and glycan, or substances containing monosaccharide or/and glycan as the substrates.

Preferably, the monosaccharide is glucose, xylose, arabinose or a combination thereof; the glycan includes cellobiose, xylobiose, sucrose, maltose, xylooligosaccharide, cellooligosaccharide, cellulose, crystalline cellulose, hemicellulose, starch, plant woody biomass or a combination thereof. More preferably, the plant woody biomass is selected from crop straw, forestry waste, energy plants or some or all of their decomposition products. Further preferably, the crop straw is selected from corn straw, wheat straw, rice straw, sorghum straw, soybean straw, cotton straw, bagasse and corncob; the forestry waste is selected from branches and leaves and sawdust; the energy plant is selected from sweet sorghum, switchgrass, miscanthus, reed or a combination thereof.

In some examples, the filamentous fungi are selected from Neurospora, Aspergillus, Trichoderma, Penicillium, Myceliophthora, Sporotrichum, Fusarium, Rhizopus, Mucor and Paecilomyces. Preferably, the Myceliophthora is selected from the group consisting of Myceliophthora thermophila and Myceliophthora heterothalica. More preferably, Myceliophthora thermophila.

In the preferred mode, the recombinant strain is Myceliophthora thermophila, and the fermentation temperature is 40-60° C., preferably 45-52, more preferably 48-50° C.

The invention also provides a method for producing ethanol. In the culture medium containing monosaccharide or/and glycan, the genetic engineering fungi obtained by the above construction method are cultured to collect ethanol from the culture.

Preferably, the monosaccharide is glucose, xylose, arabinose or a combination thereof; the glycan includes cellobiose, xylobiose, sucrose, maltose, xylooligosaccharide, cellooligosaccharide, qcellulose, crystalline cellulose, hemicellulose, starch, plant biomass or a combination thereof. Further, the plant biomass is selected from crop straw, forestry waste, energy plants or some or all of their decomposition products, more preferably, the crop straw is selected from corn straw, wheat straw, rice straw, sorghum straw, soybean straw, cotton straw, bagasse and corncob. The forestry waste is selected from branches and leaves and sawdust; the energy plant is selected from sweet sorghum, switchgrass, miscanthus, reed or a combination thereof.

In some examples, the filamentous fungi are selected from Neurospora, Aspergillus, Trichoderma, Penicillium, Myceliophthora, Sporotrichum, Fusarium, Rhizopus, Mucor and Paecilomyces. Preferably, the Myceliophthora is selected from M. thermophila and M. heterothalica; more preferably, M. thermophila. Preferably, the filamentous fungi are Myceliophthora or Trichoderma. More preferably, the Myceliophthora is selected from M. thermophila and M heterothalica. The most preferred Myceliophthora fungus is selected from M. thermophila, and more preferably, the fermentation temperature is 40-60° C., preferably 45-52° C., more preferably 48-50° C. The specific steps include culturing the recombinant fungi in a substrate; collect the culture medium and extract ethanol.

For the first time, the invention carries out genetic transformation on filamentous fungi, especially the strains of Myceliophthora, so as to enhance the ethanol production capacity of the strains. In particular, it has the ability to effectively use the fermentation substrates such as glycans or plant biomass that can not be used by ordinary strains to synthesize ethanol. In particular, using M. thermophila, through genetic transformation, ethanol can be synthesized in high yield under high temperature that ordinary strains cannot tolerate. The use of M. thermophila can realize the one-step conversion of biomass raw materials to ethanol and reduce the production cost of cellulosic ethanol. The invention verifies the above effect through experiments and has outstanding application potential and wide practical application prospects.

### BRIEF DESCRIPTION OF THE DRAWINGS

FIG. 1 shows the ethanol yield of wild-type strain, engineering strain E1 under the conditions of glucose and cellulose;

FIG. 2 shows the ethanol yield of engineering fungi E1 and E2 under the conditions of glucose and cellulose;

FIG. 3 shows the ethanol yield of engineering fungi E2 and E3 under the conditions of glucose and cellulose;

FIG. 4 shows the ethanol yield of wild-type strains, engineering strains JY144 and JY518 under the conditions of cellobiose and cellulose;

FIG. 5 shows the ethanol yield of engineering fungi E3 and E4 under the conditions of glucose and cellulose;

FIG. 6 shows the ethanol yield of engineering fungi E3 and E5 under the conditions of glucose and cellulose;

FIG. 7 shows the ethanol yield of engineering fungi E1 and E7 under the conditions of glucose and cellulose;

FIG. 8 shows the ethanol yield of engineering fungi E1 and E6 under the conditions of glucose and cellulose;

FIG. 9 shows the ethanol yield of engineering fungi E7 and E8 under the conditions of glucose and cellulose;

FIG. 10 shows the ethanol yield of engineering fungi E1, E10, E11 and E12 under the conditions of glucose and cellulose;

FIG. 11 shows the ethanol yield of engineering fungi E5, E14 and E15 under the conditions of glucose and cellulose.

FIG. 12 shows the ethanol yield of wild-type strain WT and engineering strains E17, E18 and E19 under the conditions of glucose and cellulose;

FIG. 13 shows the ethanol yield of wild-type strain WT and engineering strains E17, E18 and E19 under the conditions of cellobiose, sucrose and xylan;

FIG. 14 shows the content of residual glucose in the culture medium of wild-type strain WT and engineering strains E17, E18 and E19 after 3, 5 and 7 days of culture;

FIG. 15 shows the ethanol yield of wild-type strain, strain A1 under the conditions of glucose and cellulose;

FIG. 16 shows the ethanol yield of wild-type strain, strain A2 under the conditions of glucose and cellulose.

In the figures, Glucose represents glucose, Cellobiose represents cellobiose, Avicel represents cellulose, and WT represents the wild-type strain ATCC42464 of M. thermophila.

### DETAILED EXAMPLES

In order to further elaborate the technical means adopted by the invention and its effects, the technical schemes of the invention are further described below in combination with the preferred examples of the invention. It should be understood that these examples are only used to illustrate the invention and not to limit the scope of the invention. Those skilled in the art should understand that the details and forms of the technical schemes of the invention can be modified or replaced without departing from the spirit and scope of the invention, but these modifications or replacements fall within the protection scope of the invention.

Unless otherwise specified, the methods used in the following examples are conventional methods, such as the conditions described in <Molecular cloning: laboratory manual> (New York: Cold SpringHarbor Laboratory Press, 1989) written by Sambrook et al, or according to the conditions recommended by the manufacturer. The reagents or instruments used without the manufacturer indicated are conventional products that can be purchased through formal channels.

The percentage concentration in the example of the invention is mass percentage concentration unless otherwise specified.

### Example 1

1. Construction of Scadh1 Overexpression Vector (pAN52-Scadh1)

The expression vector was constructed with pAN52-TB-Intron (Environ Microbiol. 015.17(4):1444-62) as the skeleton. The PeIF fragment (sequence shown in SEQ ID No.1) of the promoter of eIF-5A gene (Mycoth_2297659, gene ID: 11511639) was inserted into the linearized vector pAN52-TB-Intron digested by Bgl II and Spe I to obtain the recombinant plasmid pAN52-PeIF-TtrpC-neo. Then, the fragment of Scadh1 (gene ID: 854068) encoding ethanol dehydrogenase from S. cerevisiae S288c was amplified with primers eIF-ScADH1-F/R, and recombined into the linearized vector pAN52-PeIF-TtrpC-neo digested by EcoRI and BamHI with Gibson assembly kit of NEB, to obtain the expression vector pAN52-PeIF-Scadh1 of Scadh1 encoding ethanol dehydrogenase regulated by constitutive strong promoter PeIF.

The PCR reaction system is: 2×Phanta® Max Buffer 25 μL, 10 mM dNTPs 1 μL, upstream/downstream primers 1.5 μL of each, template DNA 1 μL Phanta® Max Super-Fidelity DNA Polymerase 1 μL, double distilled water 19 μL.

The PCR reaction conditions were as follows: first, 95° C. for 60s; then 98° C. 15s, 58° C. 15s, 72° C. 2 min, 34 cycles; finally, 72° C. for 5 min and 4° C. for 10 min.

The primers used for vector construction in this example are as follows:

| SEQ ID NO. | Primer | Sequence (5′-3′) |
|---|---|---|
| 3 | eIF-5A-F | tctgcagatctttaattaactcgagCCACATCATGTAAACAGAG |
| 4 | eIF-5A-R | ggatagacatCTTGTTGTTGTTGTTGTG |
| 5 | eIF-ScADH1-F | caacaacaagATGTCTATCCCAGAAACTC |
| 6 | eIF-ScADH1-R | gatttcagtaacgttaagtggatccTTATTTAGAAGTGTCAACAACG |

### 2. The Expression Vector (pAN52-Scadh1) was Introduced into M. thermophila

M. thermophila ATCC 42464 was cultured in MM inclined medium [50×Vogel's salt 20 mL, sucrose 20 g, agar 15 g, histidine (50 mg/mL) 20 mL, fix the volume to 1 L, autoclave. The formula of 50×Vogel's salt (1 L) is: 150 g trisodium citrate (½H₂O), 250 g anhydrous KH₂PO₄, 100 g anhydrous NH₄NO₃, 10 g MgSO₄. 7H₂O, 5 g CaCl₂.2H₂O, 5 mL Microelement solution, 2.5 mL biotin (0.1 mg/mL), and fix the volume to 1 L. Microelement solution formula (100 mL): 5 g C₆H₈O. 7H₂O, 5 g ZnSO₄.7H₂O 1 g Fe (NH₄)₂(SO₄). 6H₂O, 0.25 g CuSO₄. 5H₂O, 0.05 g MnSO₄. H₂O, 0.05 g H₃BO₃, 0.05 g NaMoO₄ .2H₂O, dissolve in water, fix the volume to 100 mL] and incubate at 45° C. for 10 days.

### 2.2 Protoplast Transformation of M. thermophila

1) Mycelium Preparation

The mature spores of M. thermophila were collected with sterilized water containing 0.05% Tween 80, filtered by mirror paper, coated on MM plate covered with cellophane and cultured at 37° C. for 16 h.

2) Protoplast Preparation

Put cellophane with hyphae into 30 mL lysate (formula: 0.15 g lysase, aseptic operation, add 30 mL solution A, filter and sterilize; formula of solution A: 1.036 g potassium dihydrogen phosphate, 21.864 g sorbitol, dissolve in 90 mL deionized water, adjust the pH to 5.6 with potassium hydroxide, fix the volume to 100 mL, high temperature sterilization), lyse at 28° C. for 2 h, and shake gently every 15 min.

Then, after filtering with mirror wiping paper, centrifuge at 4° C., 2000 rpm for 10 min, discard the supernatant, add 4 mL solution B (0.735 g calcium chloride, 18.22 g sorbitol, 1mLTris. HCl (1M, pH 7.5), dissolve in 90 mL deionized water, adjust the pH to 7.6 with hydrochloric acid, fix the volume to 100 mL, and sterilize at high temperature), and centrifuge at 4° C., 2000 rpm for 10 min; discard the supernatant and add a certain volume of solution B according to 200 μL/5 μg plasmid.

3) Protoplast Transformation

Precooled 15 mL centrifuge tube, successively add 50 μL precooled PEG (12.5 g PEG6000, 0.368 g calcium chloride, 500 μL Tris. HCl (1M pH 7.5)), 10 μL plasmid pAN52-Scadh1, 200 protoplast linearized with Bgl II. Place it on ice for 20 min, add 2 mL precooled PEG, at room temperature for 5 min, add 4 mL solution B, and mix gently. Add 3 mL of the above solution into 12 mL of melted MM medium containing corresponding antibiotics, place it in the plate, culture at 45° C., and after 2 d-4 d,pick out a single mycelium under the stereomicroscope, and culture on the corresponding resistant plate.

### 2.3 Verification of Transformant of M. thermophila

(1) Genome Extraction

Genomic DNA is extracted from the transformants selected in the above transformation process by phenol chloroform method, including the following operations:

1) Add 200 mg zirconium beads and 1 mL lysis buffer (formula: 0.2MTris. HCl (pH 7.5), 0.5M NaCl, 10 mM EDTA, 1% SDS (w/V)) into the 2.0 mL distilled DNA extraction tube, and pick out the filaments of M. thermophila growing in the plate into the DNA extraction tube.

2) Place all DNA extraction tubes on the grinding aid, oscillate at the maximum speed for 30s, and repeat twice.

3) 65° C. water bath for 30 min, take out and vortex the tubes every few min during water bathing. 4) After water bathing, take out and add 80 μL 1M Tris HCl (pH 7.5) to each tube for neutralization.

5) Add 400 μL phenol:chloroform (1:1), mix up and centrifuged at 13000 rpm for 5 min.

6) Take 300 μL supernatant into a new 1.5 mL EP tube and add 600 μL 95% ethanol (DNA).

7) After incubation on ice for one hour, centrifuged at 4° C., 13000 rpm for 10 min, get white DNA precipitated to the bottom of EP tube.

8) Wash the precipitates with 400 μL 75% ethanol (DNA grade), centrifuge at 4° C., 13000 rpm for 10 min, and gently take out the supernatant.

9) Place the EP tube in a vacuum concentrator and vacuum dry the ethanol.

10) Add 50 μLddH₂O to dissolve the DNA, and determine the DNA concentration with nanodrop. After measuring the concentration, place the extracted DNA in a refrigerator at −20° C. for further PCR verification.

### 2.4 PCR Validation for M. thermophila Transformants

The genomic DNA was used as the templates, and eIF-5A-F and eIF-ScADH1-R were used as the primers for PCR validation of the transformants. The PCR products were subjected to 1% agarose gel electrophoresis (110V voltage, 30 min). The gene amplification bands were observed under the gel imaging system. It was shown that the target band of ˜2486 bp was obtained by PCR amplification under the guidance of primers eIF-5A-F and eIF-ScADH1-R. This band showed that the pAN52-PeIF-Scadh1 linearized by Bgl II was integrated into the genome of wild-type M. thermophila ATCC42464.

### 3. Determination of Ethanol Production Capacity of M. thermophila Transformants

Inoculate all the transformants verified above into 100 mL of 250 mL triangular flask, take glucose as carbon source medium (the medium formula is: 75 g glucose, 10 g yeast extract, 0.15 g KH₂PO₄ 0.15 g K₂HPO₄, 0.15 g MgSO₄. 7H₂O, 0.1 g CaCl₂ H₂O, 1 mL Microelement, 1 mL 0.1 mg/mL biotin solution, fix the volume to 1 L, high pressure steam sterilization), cellulose (Avicel) as carbon source medium, and the formula is: 75 g Avicel, 10 g yeast extract, 0.15 g KH₂PO₄, 0.15 g K₂HPO₄, 0.15 g MgSO₄. 7H₂O, 0.1 g CaCl₂. 2H₂O, 1 mL Microelement, 1 mL 0.1 mg/mL biotin solution, fix the volume to 1 L, high pressure steam sterilization. The transformed sporozoites collected with distilled water were filtered by two layers of distilled mirror paper, and the number of spores was calculated. The inoculation concentration was 2.5×10⁵/mL, the medium volume was 100 mL/bottle, cultured at 45° C. for 7 days, and the shaker speed was 150 rpm. The inoculation concentration was 2.5×10⁵ spores/mL, cultured at 45° C., 150 rpm, and sampled on the 7th day to determine the ethanol content.

1) Sample Handling:

Take 1 mL fermentation broth into a 1.5 mL centrifuge tube, centrifuge at 12000 rpm for 10 min, and take the supernatant to determine the ethanol content.

2) Determination of Ethanol Content

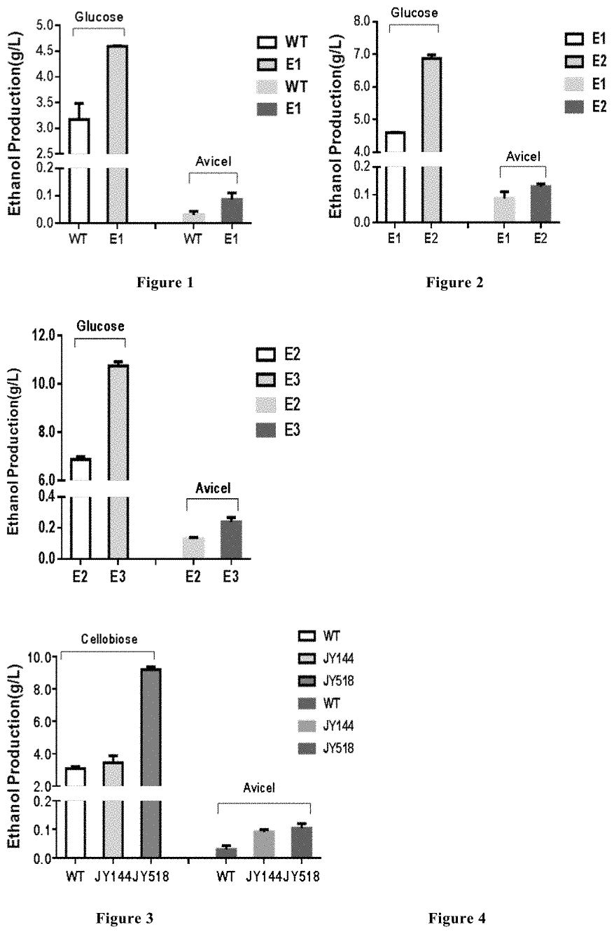

The ethanol content of the treated samples was determined by high performance liquid chromatography, in which the detector was differential detector, 5 mM H₂SO₄ was mobile phase, and the flow rate was 0.5 mL/min. The obtained strain overexpressing pAN52-PeIF-Scadh1 in wild-type strain ATCC42464 was named strain E1. The results showed that the overexpression of Scadh1 gene in wild-type strain ATCC42464 could significantly promote the production of ethanol. When glucose was used as carbon source for fermentation for 7 days, the ethanol yield of strain E1 was 4.60 g/L (as shown in FIG. 1), which was 45.1% higher than that of wild-type strain (M thermophilaATCC42464, 3.17 g/L). When fermenting with cellulose (Avivel) as carbon source for 7 days, the ethanol yield of strain E1 was 87 mg/L, which was significantly higher than that of wild-type strain (M. thermophilaATCC42464, 30.5 mg/L), which was increased by 2.8 times. The results showed that overexpression of ethanol dehydrogenase Scadh1 could significantly increase the ethanol production of M. thermophila when using glucose and cellulose (Avicel) as carbon sources.

### Example 2

1. Construction of Pdc1 Overexpression Vector (pAN52-Pdc1)

Using the genomic DNA of S. cerevisiae S288C (taxlD: 559292) as the template, pdc1 gene (gene ID: 850733) was amplified, and the 1.175 kb fragment upstream of the translation extension factor coding reading frame (Mycth_2298136) (named Ptef promoter) was used as the promoter (the nucleic acid sequence is shown in SEQ ID No.2), The two fragments were recombined into the linearized vector pAN52-TB-Intron digested by Xho I and BamHI by Gibson assembly of NEB, and the recombinant expression plasmid pAN52-tef-pdc1 of pdc1 gene was obtained.

The PCR reaction system and reaction conditions in this example are the same as those described in the vector construction in Example 1.

The primers used for vector construction in this example are as follows:

| SEQ ID NO. | Primer | Sequence (5′-3′) |
|---|---|---|
| 19 | pdc1-Gib-Ptef-F | TACCGTCAAAATGTCTGAAATTACTTTGGG |
| 25 | pdc1-Gib-Ptef-R | GATTTCAGTAACGTTAAGTGGATCCTTATTGCTTAGCGTTGG |
| | | TAG |
| 30 | Ptef-Gib-pdc1-F | TCTGCAGATCTTTAATTAACTCGAGCACCCGCCATGATTCCG |
| | | TAG |
| 31 | Ptef-Gib-pdc1-R | TTTCAGACATTTTGACGGTATTTGTGTTCTGAAGAAC |

The transformation method and verification method of subsequent target gene overexpression vector into M. thermophila are the same as those described in Example 1.

### 2. Determination of Ethanol Production Capacity of M. thermophila Transformants

The starting strain of this transformation is strain E1. The strain overexpressing pyruvate decarboxylase gene pdc1 obtained by transformation is named strain E2. Strain E2 and strain E1 were inoculated in 75 g/L glucose and 75 g/L cellulose (Avicel) medium respectively (see example 1 for the formula). The transformed sporozoites collected with distilled water were filtered by two layers of distilled mirror paper, and the number of spores was calculated. The inoculation concentration was 2.5×10⁵/mL, the volume of culture medium was 100 mL/bottle, cultured under 45° C. light for 7 days, and the rotating speed of shaker was 150 rpm.

The supernatant was centrifuged to determine the content of ethanol. The results are shown in FIG. 2: when glucose was used as carbon source, the ethanol yield of strain E2 was 6.87 g/L, which was 49.3% higher than that of the original strain E1 (4.60 g/L). When fermenting with cellulose (Avicel) as carbon source for 7 days, the ethanol yield of E1 strain was 87 mg/L and that of E2 strain was 129 mg/L, which was 48.3% higher than that of E1. It shows that overexpression of pyruvate decarboxylase gene pdc1 can increase the ethanol production of M. thermophila under the conditions of glucose and cellulose (Avicel).

### Example 3

### 1. Construction of Glt-1 Expression Vector of Glucose Transporter Gene

The glucose transporter coding gene glt-1 (NCU01633, GeneID:3872148) was amplified from the genome of Neurospora crassa. The 1372 bp fragment upstream of the glycosylaldehyde-3-phosphate dehydrogenase coding gene gpdA of M. thermophila (Mycth_2311855) (as shown in SEQ ID No.15) was used as the promoter, and the Tcbh1 fragment downstream of the cellobiose hydrolase coding gene cbh-1 of M. thermophila (Mycth_109566) was used as the Terminator (as shown in SEQ ID No.7), Glyphosate resistance gene (bar) was used as screening marker. The above fragments were recombined into the linearized vector pCAMBIA-0380 digested by BglII by Gibson assembly of NEB, so as to obtain the glt-1 overexpression vector p0380-PgpdA-1633-bar.

The PCR reaction system and reaction conditions in this example are the same as those described in Example 1.

The primers used for vector construction in this example are as follows:

| SEQ ID NO. | Primer | Sequence (5′-3′) |
|---|---|---|
| 36 | P-bar-F | AAGAGGAGTCCACCATGGTACTCGACAGAAGATGATATTG |
| 37 | P-bar-R | AACGTTAAGTTCAGATCTCGGTGACGGG |
| 39 | TtrpC-bar-F | CGAGATCTGAACTTAACGTTACTGAAATCATC |
| 41 | TtrpC-bar-R | GCTAGCGTTAACACTAGTCACCTCTAAACAAGTGTACC |
| 51 | TcbhI-F | AGCCATGGAGAGGTTTAGCACGAACCTCTCTGAAGGAG |
| 52 | TcbhI-R | GTATGATGGGTCAGTTCAG |
| 54 | 1633-F | ATGGGTCTCTTCTCGAAA |
| 55 | 1633-R | CTAAACCTCTCCATGGCT |
| 56 | PgpdA-F | AAGAGGAGTCCACCATGGTACTTGCATCGTCCCAAAGC |
| 57 | PgpdA-R | TTTCGAGAAGAGACCCATTTTGATTTCTGTGATGTGGG |

2. Analysis of Ethanol Production by Recombinant Transformant of M. thermophila

The constructed gene expression vector p0380-PgpdA-1633-bar was integrated into the genome of the starting strain, M. thermophila E2 strain (named E3 strain), and the final concentration of 100 μg/mL glyphosate was used as the screening antibiotic. See Step 2 of Example 1 for the method. The obtained transformants were verified by using primers PgpdA-F and 1633-R, the PCR system and method are shown in step 1 of example 1.

Inoculate all the verified transformants into 100 mL medium with glucose as carbon source in 250 mL triangular flask (see Step 3 of example 1 for the formula), and the inoculation concentration is 2.5×10⁵ spores/mL, 45° C., 150 rpm culture. After the sample is treated by the method described in step 3.1 of example 1, the ethanol content in the fermentation broth is determined. The method is the same as that in step 3.2 of example 1. Results as shown in FIG. 3, overexpression of glucose transporter in M. thermophila significantly promoted the production of ethanol under glucose conditions. On the 7th day, the ethanol production of strain E3 reached 10.74 g/L, which was 56.3% higher than that of control strain E2 (6.87 g/L).

When cellulose (Avicel) was used as carbon source for 7 days, the ethanol yield of strain E3 was 0.238 g/L, which was significantly higher than that of strain E2 (0.129 g/L), an increase of 84.5%. It shows that overexpression of glucose transporter/hexose transporter gene glt-1 can significantly increase the ethanol production of M. thermophila under the conditions of glucose and cellulose (Avicel).

### Example 4

In this example, firstly, JY144 strain was obtained by overexpressing the ethanol dehydrogenase gene Scadh1 in the wild-type strain ATCC42464. Then, the genome editing technology based on CRISPR/Cas9 (Qian Liu, et al. Development of a genome-editing CRISPR/Cas9 system in thermophilic fungal Myceliophthora species and its application to hyper-cellulase production strain engineering. Biotechnol Biofuels 2017, 10:1) is adopted, the cellobiose transporter coding genes cdt-1 and cdt-2 were fixed-point integrated into the ldh-1 and ldh-2 loci of the genome of strain JY144 respectively. The obtained strain was named JY518 to achieve the effect of overexpression of the target gene and knockout of the metabolic branch at the same time. The specific process is as follows:

1. Construction of Scadh1 Overexpression Vector (pAN52-Scadh1)

The expression vector was constructed with pAN52-TB-Intron (Environ Microbiol. 015.17(4):1444-62) as the skeleton. The Ptef fragment of tef gene promoter (SEQ ID No.1) was inserted into the linearized vector pAN52-TB-Intron digested by BglII and Spe I to obtain the recombinant plasmid pAN52-MtTef-TtrpC-neo. Then, the fragment of Scadh1 (Gene ID: 854068) encoding ethanol dehydrogenase from S. cerevisiae S288C was amplified with primersTef-ScADH1-F/R, and recombined into the linearized vector pAN52-MtTef-TtrpC-neo digested by EcoRI and BamHI with Gibson assembly kit of NEB to obtain the expression vector pAN52-MtTef-Scadh1. The transformation and identification methods of M. thermophila are the same as those in step 2 of Example 1.

2. Construction of sgRNA Expression Frame Vector

The protopacer (eg. The target site) of the target genes ldh-1 (Mycth_38939) and ldh-2 (Mycth_110317) was designed by software sgRNACas9 tool. The sequence sgRNA promoter, protopacer and sgRNA were connected by fusion PCR, and the sgRNA expression frame vector was constructed by gene overlap extension (SOE). The PCR reaction system and reaction conditions were the same as those in Example 1.

sgRNA expression plasmids U6p-ldh1-sgRNA and U6p-ldh2-sgRNA were obtained by SOE-PCR amplification, and their sequences were shown in SEQ ID No.12 and SEQ ID No.9, respectively.

### 3. Construction of Donor DNA Vector

Using the 1372 bp fragment upstream of glycosylaldehyde-3-phosphate dehydrogenase coding gene gpdA (Mycth_2311855) of M. thermophila as the promoter of cdt-1 (SEQ ID No.15), and the genome of M. thermophila as the template, and mediated by primers, the cellobiose transporter coding gene cdt-1 of Neurospora crassa (NCU00801, GeneID:3879950) and about 900 bp upstream/downstream homologous fragment of ldh-1 gene were amplified by PCR. Then, the Gibson assembly of NEB was used to recombine them into the linearized vector pAN52-TB-Intron digested by SpeI and EcoRV, and the obtained vector was sequenced. Donor DNA fragment donor-cdt1 that the gene cdt-1 regulated by promoter PgpdA was obtained, and its sequence was shown in SEQ ID No.16

Using the genome of Neurospora crassa as a template, and mediated by primers, the cellobiose transporter coding gene cdt-2 (NCU08114, GeneID: 3880022) of Neurospora crassa was amplified by PCR. The 730 bp fragment Ppdc upstream of pyruvate decarboxylase encoding reading frame pdc (Mycth_112121) was used as the promoter of cdt-2 (as shown in SEQ ID No.17), glyphosate resistance gene Bar was used as a screening marker, and about 1000 bp homologous fragment upstream/downstream of ldh-2 gene, the Gibson assembly of NEB was used to recombine them into the linearized vector pAN52-TB-Intron digested by SpeI and EcoRV, and the obtained vector was sequenced. The donor DNA fragment donor-cdt2 of cdt-1 gene which expression was under the regulation of promoter PgpdA was obtained, and the sequence is shown in SEQ ID No.20.

The PCR reaction system and reaction conditions in this example are the same as those described in the construction of vector in Example 1.

The primers used for vector construction in this example are as follows:

| SEQ ID NO. | Primer | Sequence (5′-3′) |
|---|---|---|
| 43 | Tef-F | GCAGATCTCATGTACCTTGACGTCCTCCGAG |
| 44 | Tef-R | AAACACTAGTTCTGAAGAACGAAACTGGCGACT |
| 45 | Tef-ScADH1-F | TTTAAACGATATCGAATTCGAGCATACAATCAACTATCTC |
| 46 | Tef-ScADH1-R | GATTTCAGTAACGTTAAGTGCTTTAAAATTTGTATACAC |
| 58 | cdt1-F | TCACAGAAATCAAAACTAGAATGTCGTCTCACGGCTC |
| 59 | cdt1-R | TTAAGTGGATCCGAATTCGATATCCTAAGCAACGATAGCTTC |
| | | GGAC |
| 60 | 38939-up-F | AAGAGGAGTCCACCATGGTACAGGATATTGATAACACC |
| 61 | 38939-up-R | CGATCTCTAGAAGTCCTCGTCCCGAGCCAAACACTTGG |
| 62 | 38939-down-F | CAGGTACACTTGTTTAGAGGCCGCTGGACAGGTTCGTG |
| 63 | 38939-down-R | GCTAGCGTTAACACTAGTCAGCCCATCTGTTGTTAACC |
| 64 | pAN52-F | ACGAGGACTTCTAGAGATCGTTGCATCGTCCCAAAGC |
| 65 | TtrpC-bar-R2 | CCTCTAAACAAGTGTACCTG |
| 66 | Ppdc-F2 | TCAAGTCCAGCATAGCGAC |
| 67 | TtrpC-bar-R2 | CCTCTAAACAAGTGTACCTG |
| 68 | 110317-up-F | AAGAGGAGTCCACCATGGTACGTCAGGTGTCTGTTCTC |
| 69 | 110317-up-R | GTCGCTATGCTGGACTTGAGTACGCAGTCGTCACGCC |
| 70 | 110317-down-F | CAGGTACACTTGTTTAGAGGTATTCCGGGACGTAGTGC |
| 71 | 110317-down-R | GCTAGCGTTAACACTAGTCACTCCAACTCGCTGAGGAG |

2. Determination of the Ability of the M. thermophila Transformants to Produce Ethanol from Cellobiose

Firstly, the vector pAN52-MtTef-Scadh1 was transferred into the genome of wild-type ATCC42464 of M. thermophila, and the obtained strain overexpressing Scadh1 gene was named as JY144 strain. Then, taking JY144 strain as the starting strain, cdt-1 and cdt-2 expression vectors, Cas9 fragment, U6p-ldh-1-sgRNA, U6p-ldh-2-sgRNA were transformed into JY144 strain, and the final concentration of glyphosate was 100 μg/mL as the screening antibiotic. The method is shown in step 2 of Example 1. The transformants were simultaneously verified by primer pairs 38939-up-F/38939-down-R and 110317-up-F/110317-down-R.

Inoculate the verified correct transformants into the medium with cellobiose and cellulose (Avicel) as carbon sources (see step 3 of Example 1 for the formula, only replace glucose with cellobiose/cellulose), and the inoculation concentration is 2.5×10⁵ spores/mL, 45° C., 150 rpm culture. After the sample is treated by the method described in step 3.1 of Example 1, the ethanol content in the fermentation broth is determined. The method is the same as that described in Example 1.

Results as shown in FIG. 4, under the condition of cellobiose as carbon source, the ethanol yield of JY144 strain was 3.45 g/L, which was higher than that of wild-type strain (3.08 g/L). Based on JY144 strain, cdt-1 and cdt-2 were overexpressed and ldh-1 and ldh-2 genes were inactivated at the same time. The obtained strain was named JY518. When cellobiose was used as carbon source, the ethanol yield of JY518 on the 7th day was 9.20 g/L, which was 1.67 times higher than that of the original strain JY144, which was 3.45 g/L.

When cellulose (Avicel) was used as carbon source for fermentation for 7 days, the ethanol yield of JY518 strain was 0.104 g/L, which was higher than that of the starting strain JY144 (0.0914 g/L), increased by 13.8%, while the ethanol yield of wild-type strain (ATCC42464) was 0.0305 g/L. The results showed that overexpression of cellobiose transporters cdt-1 and cdt-2 could increase the ethanol production of M. thermophila under cellobiose and cellulose conditions.

### Example 5

This example mainly aims at the negative regulatory genes lactate dehydrogenase Ldh-2 (Mycth_110317) and mannitol 1-phosphate dehydrogenase Mpd (Mycth_2310298), and adopts the genome editing technology based on CRISPR/Cas9 (Biotechnol Biofuels 2017, 10:1) inactivate the target gene to knock out the metabolic branch and promote ethanol synthesis. 1. Vector Construction

(1) Construction of sgRNA Expression Frame

The protospacer (eg. The target site) of target genes ldh-2 (Mycth_110317) and mpd (Mycth_2310298) was designed by the softwares gRNACas9 tool. The sgRNA promoter, protopacer and sgRNA were connected by fusion PCR, and the sgRNA expression frame vector was constructed by gene overlap extension (SOE).

The PCR reaction system and reaction conditions are as shown in Example 1.

sgRNA expression plasmids U6p-ldh-2-sgRNA and U6p-mpd-sgRNA were obtained by SOE-PCR amplification, and their sequences are shown in SEQ ID No.9 and SEQ ID No.26, respectively.

(2) Construction of donor DNA vector In the invention, the donor DNA fragments are respectively connected to the plasmid PPk2BarGFP linearized by restriction endonuclease Xbal and EcoRV by Gibson assembly method from the homologous fragment of about 600 bp upstream/downstream of the target gene and the PtrpC-bar fragment of glyphosate resistance gene expression frame, and finally construct the donor DNA fragments donor-ldh-2 and donor-mpd. Their nucleic acid sequences are shown in SEQ ID No.27 and SEQ ID No.28, respectively.

The PCR reaction system and reaction conditions in this example are the same as those described in Example 1.

The primers used for vector construction in this example are as follows:

| SEQ ID | | |
|---|---|---|
| NO. | Primer | Sequence (5′-3′) |
| 76 | 110317UP-F | AGTTTATGTGGAAAATGGCA |
| 77 | 110317UP-R | CTCGTAGTCGCCGACGGACTATCCGTTGGGCGGAGCAT |
| 78 | 110317Down-F | CCAACGGATAGTCCGTCGGCGACTACGAGGACTGCG |
| 79 | 110317Down-R | TACGTGCCGACCCTCGAGAC |
| 80 | Cas9-F | TCCTCCGAGGTTCGACATCAGGGTT |
| 81 | Cas9-R | CTCTAAACAAGTGTACCTGTGCAT |
| 82 | 110317sgRNA-b | ACCTATCAAGGAGCAGCGAGGAAAGAAAGAAAAGAAGAGG |
| 83 | 110317sgRNA-c | TGCTCCTTGATAGGTTAGGTTTTAGAGCTAGAAATAGCAA |
| 84 | 110317doner-bar-F | acggatagtcATATTGAAGGAGCACTTTTTG |
| 85 | 110317doner-bar-R | gtcgccgacgGATCACTAGTACAGGATTC |
| 86 | KO110317YZ-F | CGGATTCAAGATGCCTCAGCCA |
| 87 | KO110317YZ-R | GACCGGGTTGGTGGCCACGA |
| 88 | 2310298-up-F | CCTGACTGTATAAGAGAAATG |
| 89 | 2310298-up-R | AGCGACCGGAGTAAGCTCCCGCCGTGGCAATTTCCCGAAC |
| 90 | 2310298-Down-F | AAATTGCCACGGCGGGAGCTTACTCCGGTCGCTGTCA |
| 91 | 2310298-Down-R | CCAATGAAGCGTTCCTTACGGCT |
| 92 | sgRNA-a | AGGATCGGTGGAGTGAAGTTCG |
| 93 | sgRNA-b | ACGGAGCAGGTAACCAAGTCGAGGAAAGAAAGAAAAGAAG |
| 94 | 2310298sgRNA-c | GACTTGGTTACCTGCTCCGTGTTTTAGAGCTAGAAATAGC |
| 95 | 2310298sgRNA-d | AAAAAAAGCACCGACTCGGTGCC |
| 96 | KO2310298YZ-F | TGACTGACCGTGATGATTTCCAG |
| 97 | KO2310298YZ-R | AGCCTAACGTCGAGACCGGCGT |

2. Determination of Ethanol Production Capacity of M. thermophila Transformants

Cas9 expression frame, sgRNA expression plasmids U6p-ldh-2-sgRNA (SEQ ID No.9) and U6p-mpd-sgRNA (SEQ ID No.26), and donor DNA fragments donor-ldh-2(SEQ ID No.27) and donor-mpd (SEQ ID No.28) were mixed in equal proportion and co-transformed into protoplast cells of M thermophila strain E3. Cas9 protein was mediated by gRNA, the target site is identified and cut by pairing the target sequence with the DNA strand of ldh-2 (Mycth_110317) and mpd (Mycth_2310298) on the host cell genome, and then the donor DNA fragment is homologously recombined with the sequences on both sides of the target site, so as to achieve the purpose of editing the genome. The transformants are screened by adding glyphosate to the plate. The transformation method and verification method of introducing the target gene fragment into M. thermophila are the same as those described in Example 1.

The transformant (named E4 strain) with simultaneous knockout of lactate dehydrogenase gene ldh-2 and 1-phosphate mannitol dehydrogenase gene mpd was obtained. Strain E4 and strain E3 were inoculated in 75 g/L glucose and 75 g/L cellulose (Avicel) medium, respectively (see example 1 for the formula). The transformed sporozoites collected with distilled water were filtered by two layers of distilled mirror paper, and the number of spores was calculated. The inoculation concentration was 2.5×10⁵ spores/mL, the medium volume was 100 mL/bottle, cultured at 45° C. for 7 days, and the shaker speed was 150 rpm.

The supernatant was centrifuged to determine the content of ethanol. The results are shown in FIG. 5: the ethanol yield of strain E4 increased from 10.74 g/L of E3 to 13.70 g/L, an increase of 27.6%, indicating that the knockout of ldh-2 and mpd genes is helpful to improve the ethanol yield of M. thermophila under glucose conditions.

When cellulose was used as carbon source for fermentation for 7 days, the ethanol yield of strain E4 was 0.265 g/L, which was higher than that of starting strain E3 (0.238 g/L), increased by 11.3%. The results showed that knockout of lactate dehydrogenase gene and 1-phosphate mannitol dehydrogenase gene could increase the ethanol production of M. thermophilaon using cellulose as the carbon source.

### Example 6

This example mainly aims at the negative regulatory gene cytoplasmic malate dehydrogenase Mdh (Mycth_2315052, GeneID:11512768), and adopts the genome editing technology based on CRISPR/Cas9 inactivate the target gene, reduce the shuttle of reducing force of cytoplasmic NADH to mitochondria, and then promote ethanol synthesis.

1. Construction of sgRNA Expression Frame

The protopacer (eg. The target site) of the target gene mdh (Mycth_2315052) is designed through the software sgRNACas9 tool. The sgRNA promoter, protopacer and sgRNA were connected by fusion PCR, and the sgRNA expression frame vector was constructed by gene overlap extension (SOE). The specific operations were as follows: firstly, the promoter and protopacer DNA fragments were amplified with primers sgRNA-a and 2315052 sgRNA-b; protopacer and scaffold fragments were amplified with primers 2315052 sgRNA-c and sgRNA-d, and then the full length of sgRNA expression frame was amplified with primers sgRNA-a and sgRNA-d using the two DNA fragments as templates amplified as above mentioned. The PCR reaction system and reaction conditions are as shown in example 1.

sgRNA expression plasmid U6p-mdh-sgRNA was obtained by SOE-PCR amplification, and its sequence is shown in SEQ ID No.32.

### 2. Construction of Donor DNA Vector

In the invention, the donor DNA fragment is respectively composed of the upstream homologous arm of the target gene, the PtrpC-bar fragment of the glyphosate resistance gene expression frame and the downstream homologous arm, which are connected together by fusion PCR, and finally constructed into the donor DNA fragment donor-mdh. The nucleic acid sequence is shown in SEQ ID No.33. The construction process was as follows: firstly, the upstream homologous arm DNA was amplified with primers 2315052donor-up-F and 2315052donor-up-R, the glyphosate resistance gene expression frame PtrpC-bar fragment was amplified with primers 2315052donor-bar-F and 2315052donor-bar-R, and the downstream homologous arm was amplified with 2315052donor-down-F and 2315052donor-down-R. Then, the upstream homologous arm and PtrpC-bar fragment were used as templates at the same time, and the Fusion DNA fragment of the upstream homologous arm and the glyphosate resistance gene expression frame was amplified with primers 2315052donor-up-F and 2315052donor-bar-R. Then, the Fusion DNA fragment and the downstream homologous arm were used as templates at the same time, and the full-length donor DNA fragment donor-mdh was amplified with primers 2315052donor-up-F and 2315052donor-down-R. Finally, the donor DNA sequence was proved to be correct by gene sequencing.

Construction of cas9 expression frame as published by the inventor (biotechnol biofuels 2017, 10:1), the nucleic acid sequence is shown in SEQ ID No.29.

The PCR reaction system and reaction conditions in this example are the same as those described in Example 1.

The primers used for vector construction in this example are as follows:

| SEQ ID NO. | Primer | Sequence (5′-3′) |
|---|---|---|
| 98 | 2315052doner-up-F | GGGTGGTGTGCATGGGGCCGTCT |
| 99 | 2315052doner-up-R | AAAGTGCTCCTTCAATATGCAGCAGGGTCAGCCGGGTCT |
| 100 | 2315052doner-Bar-F | CGGCTGACCCTGCTGCATATTGAAGGAGCACTTTTTGGGC |
| 101 | 2315052doner-Bar-R | GGTCATGCCGGGCTTTTAAGAAACTTTATTGCCAA |
| 102 | 2315052doner-Down-F | GGCAATAAAGTTTCTTAAAAGCCCGGCATGACCCGTGATGA |
| 103 | 2315052doner-Down-R | CGTACTGAAGCTCTTCCTCTATCCT |
| 104 | 2315052 sgRNA-b | TTGGCGGGCAGGTAGCCGAGGAAAGAAAGAAAAGAAGAG |
| 105 | 2315052 sgRNA-c | CTACCTGCCCGCCAACGAGTTTTAGAGCTAGAAATAGCAA |
| 106 | sgRNA-a | AGGATCGGTGGAGTGAAGTTCG |
| 107 | sgRNA-d | AAAAAAAGCACCGACTCGGTGCC |

3. Determination of Ethanol Production Capacity of M. thermophila Transformants

Cas9 expression frame, sgRNA expression plasmid U6p-mdh-sgRNA and donor DNA fragment donor-mdh were mixed in equal proportion and co-transformed into protoplast cells of strain E3 and strain E1. Cas9, mediated by gRNA, identified the target site by pairing the target sequence with the DNA strand of mdh on the host cell genome, and then homologous recombination occurred between the donor DNA fragment and the sequences on both sides of the target site. In order to achieve the purpose of gene editing, the transformants were screened by adding glufosinate to the plate. The transformation method and verification method of introducing the target gene fragment into M thermophila are the same as those described in Example 1, and strain E5 (starting strain E3) and strain E7 (starting strain E1) are obtained respectively.

Strain E5 and its starting strains E3, E7 and its starting strain E1 were inoculated into 75 g/L glucose and 75 g/L cellulose (Avicel) medium, respectively (see example 1 for the formula). The transformed sporozoites obtained were collected with distilled water and filtered by two layers of distilled mirror paper. The number of spores was calculated. The inoculation concentration was 2.5×10⁵/mL, the volume of medium was 100 mL/bottle, cultured at 45° C. for 7 days, and the rotating speed of shaker was 150 rpm. The supernatant was centrifuged to determine the content of ethanol.

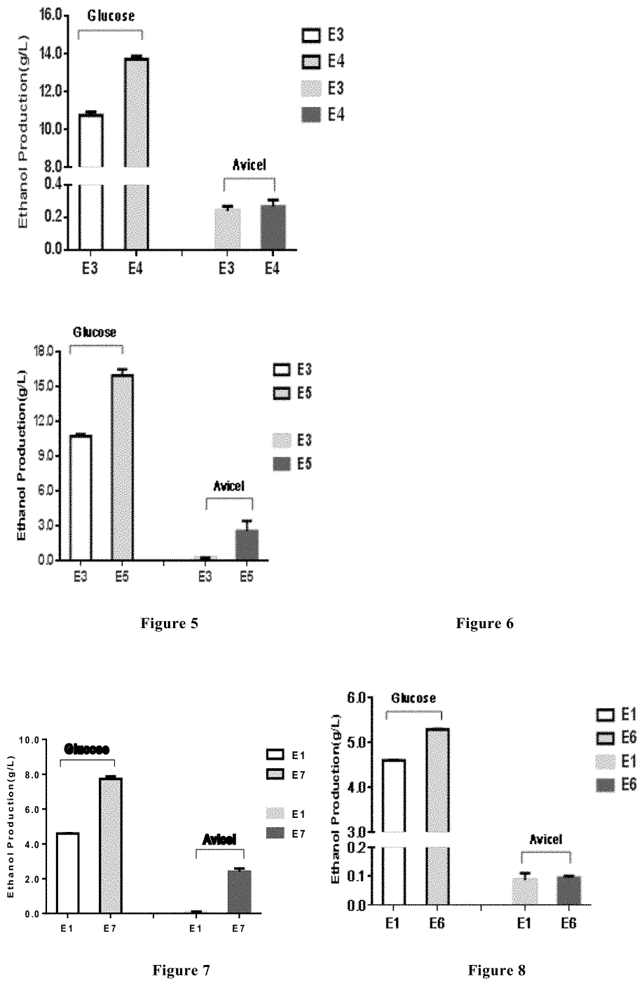

The results are shown in FIG. 6: when glucose is used as carbon source, the ethanol yield of strain E5 reaches 15.96 g/L, which is 48.6% higher than that of the original strain E3 (10.74 g/L). When cellulose (Avicel) was used as carbon source, the ethanol yield of strain E5 was 2.568 g/L, which was significantly higher than that of original strain E3 (0.238 g/L), which was increased by 9.8 times. It shows that the knockout of cytoplasmic malate dehydrogenase mdh can significantly increase the ethanol production of M. thermophila under the conditions of glucose and cellulose (Avicel)

Similarly, as shown in FIG. 7: when glucose is used as carbon source, the ethanol yield of strain E7 reaches 7.73 g/L, which is 68.04% higher than that of control strain E1 (the yield is 4.60 g/L). When cellulose (Avicel) was used as carbon source, the ethanol yield of strain E7 was 2.41 g/L, which was significantly higher than that of strain E1 (0.087 g/L), increased by 26.7 times. It shows that the knockout of cytoplasmic malate dehydrogenase gene mdh can increase the content of cytoplasmic NADH, so as to improve the ethanol production capacity under the conditions of glucose and cellulose.

### Example 7

This example mainly applies CRISPR/dCas9 technology to down-regulate the transcription level of cytochrome C oxidase coding gene (Mycth_2312390, GeneID:11509370), a key gene of mitochondrial respiratory chain, so as to promote the synthesis of ethanol by M. thermophila. The details are as follows.

### 1. Construction of Expression Vector

(1) Amplification of dCas9 Protein Coding Sequence

dCas9 protein mutates two key domains in Cas9, RuvC and HNH (D10A and H840A), making it lose the activity of cutting DNA. Mutation method: use primers to introduce the two mutation sites (D10A and H840A) at the same time, take the vector p0380-bar-Ptefl-Cas9-TtprC as the template (Biotechnol Biofuels 2017, 10:1), amplify the pre/post Cas9 sequences with primers respectively, and connect the above fragments together by fusion PCR to obtain dCas9 nucleic acid fragment, as shown in SEQ ID No.38.

KRAB domain is a protein-protein interaction region, which can bind to a variety of synergistic transcription inhibitors, so that KRAB zinc finger protein can play a transcription inhibitory function dependent on DNA binding as a transcription factor and/or transcription regulator. The KRAB domain used in the invention comes from human zinc finger protein 10 (Homo sapiens zinc finger protein 10), which is synthesized artificially after codon optimization as SEQ ID No.40.

dCas9 was connected with KRAB nucleic acid fragment by fusion PCR to obtained Cas9KRAB fragment. After enzyme digestion with PmeI and PacI, it was connected to the linearized vector p0380-bar-Ptefl-Cas9-TtprC digested by the same enzymes, and the recombinant expression plasmidp0380-bar-Ptefl-dCas9KRAB-TtprC was obtained.

(2) Construction of sgRNA Expression Frame

The protopacer in the upstream promoter region of the target gene Mycth_2312390 which is the target site was designed by software sgRNACas9 tool. The sequence sgRNA promoter, protopacer and sgRNA were connected by fusion PCR, and the sgRNA expression frame vector U6p-2312390-sgRNA was constructed by gene overlap extension (SOE). The nucleic acid sequence is shown in SEQ ID No.42.

The PCR reaction system and reaction conditions in this example are the same as those described in Example 1.

The primers used for vector construction in this example are as follows:

| SEQ ID NO. | Primer | Sequence (5′-3′) |
|---|---|---|
| 120 | dCas9-a | AAGCTTATGGACCCCAAGAAGAAACGCAAGGTTGATAA |
| | | GAAGTACTCCATCGGCCTCGCCAT |
| 121 | dCas9-b | TGAGGGACGATAGCATCGACATCGTAATCGGA |
| 122 | dCas9-c | CGATGTCGATGCTATCGTCCCTCAGTCCTTCC |
| 123 | dCas9-d | AGCGGCCGCCACCTTCCTCTTTT |
| 124 | dCas9-KRAB-F | GGGTTTAAACATGGACCCCAAGAAGAAACGCAA |
| 125 | dCas9-KRAB-R | TAGCATCCATAGCGGCCGCCACCTTCCT |
| 126 | KRAB-F | CGGCCGCTATGGATGCTAAGTCACTAACT |
| 127 | KRAB-R | CCTTAATTAATGCAGTCTCTGAATCAGGATGGGT |

2. Transformation of M. thermophila and Identification of Transformants

The p0380-bar-Ptefl-dCas9KRAB-TtprC vector was linearized by EcoRV and transformed into the starting strain E1 in the same amount as U6p-2312390-sgRNA (see Example 1 for construction and transformation methods). After obtaining the transformants, the transformants were identified by primers dCas9-KRAB-F and KRAB-R(verifying that dCas9KRAB was integrated into the genome), 2312390Sg-c and 2312390Sg-d (verifying that U6p-2312390-sgRNA was integrated into the genome). The obtained transformants were verified by PCR. The PCR system and method are shown in example 1. The transformants successfully transformed into p0380-bar-Ptefl-dCas9KRAB-TtprC and 2312390SgRNA were named strain E6.

3. Determination of Ethanol Production Capacity of M. thermophila Transformants

Strain E6 and starting strain E1 were inoculated into the culture medium of 75 g/L glucose and 75 g/L cellulose (see example 1 for the formula). The transformed sporozoites collected with distilled water were filtered by two layers of distilled mirror paper, and the number of spores was calculated. The inoculation concentration was 2.5×10⁵/ mL, the volume of culture medium was 100 mL/bottle, cultured at 45° C. for 7 days, and the rotating speed of shaker was 150 rpm.

The supernatant was centrifuged to determine the ethanol content. The results are shown in FIG. 8. When glucose was used as carbon source, the ethanol yield of transformant E6 reached 5.29 g/L, which was 15% higher than that of starting strain E1 (4.60 g/L). When cellulose (Avicel) was used as carbon source for fermentation for 7 days, the ethanol yield of strain E6 was 0.094 g/L, which was higher than that of starting strain E1 (0.087/L), increased by 8%, indicating that the down-regulation of respiratory chain intensity can improve the ethanol yield of M. thermophila on glucose and cellulose (Avicel).

### Example 8

In order to improve the level of NADH in the cytoplasm, the glycerol-3-phosphate dehydrogenase gene gpd(Mycth_2313529, GeneID:11508057) in the ethanol synthesis strain of M thermophila was knocked out to further improve the ethanol synthesis efficiency of the transformants.

This example constructs the sgRNA expression frame U6p-gpd-sgRNA (as shown in SEQ ID No.34) and its donor DNA expression vector donor-gpd (as shown in SEQ ID No.35) according to the method described in Example 6.

The PCR reaction system and reaction conditions in this example are the same as those described in Example 1.

The primers used for vector construction in this example are as follows:

| SEQ ID NO. | Primer | Sequence (5′-3′) |
|---|---|---|
| 110 | 2313529doner-up-F | TGGGACGGCGAGAGCGACGGGT |
| 111 | 2313529doner-up-R | GCGTCGAGAAGGCAGAGTCTGTGGATCGTGC |
| 112 | 2313529doner- | AGACTCTGCCTTCTCGACGCCGTCAGGG |
| | Down-F | |
| 113 | 2313529doner- | TAGCGCCAGTTGCGTGCCGAGGT |
| | Down-R | |
| 114 | 2313529sgRNA-b | CGGGGATTGTGACATTCTCCGAGGAAAGAAAGAAAAGAAG |
| 115 | 2313529sgRNA-c | GGAGAATGTCACAATCCCCGGTTTTAGAGCTAGAAATAGC |
| 116 | sgRNA-a | AGGATCGGTGGAGTGAAGTTCG |
| 117 | sgRNA-d | AAAAAAAGCACCGACTCGGTGCC |
| 118 | KO2313529YZ-F | CGCTCTCATTCCCCCTCGCAG |
| 119 | KO2313529YZ-R | ATATACTCGCAAATCAGCTCGA |

2. Determination of Ethanol Production Capacity of M. thermophila Transformants

Cas9 expression frame, sgRNA expression plasmid U6p-gpd-sgRNA and donor DNA fragment donor-gpd were mixed in equal proportion and co-transformed into protoplast cells of M. thermophila E7 strain (see Example 6 for the construction process). Cas9 identified the target site through the pairing of the target sequence with the DNA strand of gpd on the host cell genome mediated by gRNA, and then the donor DNA fragment was homologously recombined with the sequences on both sides of the target site, so as to achieve the purpose of editing the genome. The transformation method and verification method of introducing the target gene fragment into M. thermophila are the same as those described in example 1. The transformant that successfully knocked out the gpd gene in strain E7 is named strain E8.

Strain E8 and its starting strain E7 were inoculated in 75 g/L cellulose (Avicel) medium and 75 g/L glucose medium (See example 1 for the formula). The transformed sporozoites collected with distilled water were filtered by two layers of distilled mirror paper, and the number of spores was calculated. The inoculation concentration was 2.5×10⁵ spores/mL, the medium volume was 100 mL /bottle, cultured at 45° C. for 7 days, and the shaker speed was 150 rpm.

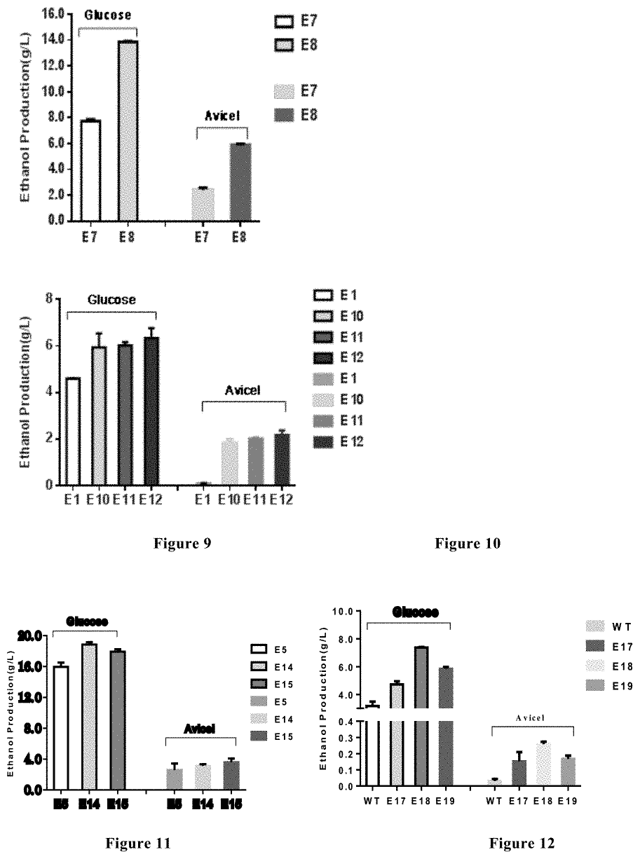

The supernatant was centrifuged to determine the ethanol content. The results are shown in FIG. 9: on the 7th day, under the condition of glucose as carbon source, the ethanol yield of strain E8 reached 13.84 g/L, which was 79% higher than that of control strain E7 (the yield was 7.73 g/L). Under the condition of cellulose (Avicel), the ethanol yield of strain E8 was significantly increased to 5.91 g/L, which was 145.2% higher than that of strain E7 (2.41 g/L). It shows that inactivation of cytoplasmic glycerol-3-phosphate dehydrogenase Gpd can significantly improve the ethanol production of M. thermophila under the conditions of cellulose (Avicel) and glucose.

### Example 9

This example mainly aims at the NADH dehydrogenase gene nde outside the negative regulation gene (Mycth_2304268, Gene ID: 11507602 encodes nde1, Mycth_2304512, Gene ID: 11507705 encodes nde2), and adopts the genome editing technology based on CRISPR/Cas9. Inactivate the target gene, reduce the transmission of cytoplasmic NADH reducing force to mitochondria, and then promote ethanol synthesis.

1. Construction of sgRNA Expression Frame

The protopacer (eg. The target site) of target genes nde1 (Mycth_2304268) and nde2 (Mycth_2304512) was designed by software sgRNACas9 tool. The sequence sgRNA promoter, protopacer and sgRNA were connected by fusion PCR, and the sgRNA expression frame vector was constructed by gene overlap extension (SOE).

The PCR reaction system and reaction conditions are shown in example 1.

sgRNA expression plasmids U6p-nde1-sgRNAand U6p-nde2-sgRNA were obtained by SOE-PCR amplification. The sequences are shown in SEQ ID No.144 and SEQ ID No.146, respectively. The detailed construction process is shown in example 6

### 2. Amplification of Donor DNA

In the invention, the donor DNA fragment consists of about 700 bp homologous fragment upstream/downstream of the target gene and PtrpC-bar fragment of glyphosate resistance gene expression frame respectively. The full-length linearized donor DNA fragments donor-nde1 and donor-nde2 are obtained by fusion PCR, and their nucleic acid sequences are shown in SEQ ID No.145 and SEQ ID No.147, respectively. The detailed construction process is shown in example 6.

The PCR reaction system and reaction conditions in this example are the same as those described in Example 1.

The primers used for vector construction in this example are as follows:

| SEQ ID NO. | Primer | Sequence (5′-3′) |
|---|---|---|
| 148 | sgRNA-a | AGGATCGGTGGAGTGAAGTTCG |
| 149 | 2304268sgRNA-b | CTGATAGAAGCCAATCGAGGAAAGAAAGAAAAGAAGA |
| 150 | 2304268sgRNA-c | GATTGGCTTCTATCAGGGCTGTTTTAGAGCTAGAAATAGC |
| 151 | sgRNA-d | AAAAAAAGCACCGACTCGGTGCC |
| 152 | 2304268donor-up-F | CGGGCTCAGCCGGAACTTGCTACC |
| 153 | 2304268donor-up-R | CCTTCAATATGGCTAGCAACGGGGTGAAG |
| 154 | 2304268donor-Bar-F | GTTGCTAGCCATATTGAAGGAGCACTTTTTG |
| 155 | 2304268donor-Bar-R | GGGATGACCGGATCACTAGTACAGGATTC |
| 156 | 2304268donor-Down-F | ACTAGTGATCCGGTCATCCCCGCTCCTT |
| 157 | 2304268donor-Down-R | GATCAAGGGGTTTGGCATGAG |
| 158 | sgRNA-a | AGGATCGGTGGAGTGAAGTTCG |
| 159 | 2304512sgRNA-b | TCTGGCGGAGGATGGTCGAGGAAAGAAAGAAAAGAAG |
| 160 | 2304512sgRNA-c | ACCATCCTCCGCCAGAAGAGTTTTAGAGCTAGAAATAGCA |
| 161 | sgRNA-d | AAAAAAAGCACCGACTCGGTGCC |
| 162 | 2304512donor-up-F | TCCTCCTCCCTCTCTCAGGCGCC |
| 163 | 2304512donor-up-R | CCTTCAATATAGTTGCGCGGGGAGATGAC |
| 164 | 2304512donor-Bar-F | CCGCGCAACTATATTGAAGGAGCACTTTTTG |
| 128 | 2304512donor-Bar-R | CCGGATCTCGGATCACTAGTACAGGATTC |
| 129 | 2304512donor-Down-F | ACTAGTGATCCGAGATCCGGGGTGACACC |
| 130 | 2304512donor-Down-R | CAGTCGCCCACGGCCCAGATGT |

3. Determination of Ethanol Production Capacity of M. thermophila Transformants

After Cas9 expression frame, sgRNA expression plasmid U6p-nde-sgRNA and donor DNA fragment donor-nde are mixed in equal proportion and co-transformed into protoplast cells of M thermophila E1 strain, Cas9 recognizes the target site by pairing the target sequence with the DNA strand of nde on the host cell genome mediated by gRNA, and then homologous recombination occurs between the donor DNA fragment and the sequences on both sides of the target site, so as to achieve the purpose of genome editing. Transformants were screened by adding glufosinate to the plate. The transformation method and verification method of introducing the target gene fragment into M thermophila are the same as those described in Example 1,

The starting strain of this transformation is strain E1. The strain obtained by knocking out NADH dehydrogenation gene nde1 is named strain E10, the strain obtained by knocking out nde2 is named strain E11, and the strain knocked out nde1 and nde2 is named strain E12. Strains E10, E11, E12 and E1 were inoculated in 75 g/L glucose medium and 75 g/L cellulose (Avicel) medium respectively (see example 1 for the formula). The ethanol content was measured after 7 days of culture. The results are shown in FIG. 10: in glucose medium, the ethanol yield of strains E10, E11 and E12 were 5.94 g/L, 6.02 g/L and 6.33 g/L respectively, which were higher than that of the original strain E1 (4.60 g/L), 29.1%, 30.9% and 37.6% higher than that of E1 respectively. Under the condition of using cellulose (Avicel) as carbon source, the results are shown in FIG. 10: the ethanol yields of strains E10, E11 and E12 are 1.82 g/L, 2.01 g/L and 2.17 g/L respectively, which are higher than those of the starting strain E1 (0.087 g/L), 19.92 times, 22.10 times and 23.94 times higher than those of E1 respectively. The results showed that the knockout of NADH dehydrogenase could increase the content of NADH in the cytoplasm of M. thermophila, and then increase the yield of ethanol under the conditions of glucose and cellulose.

### Example 10

In vivo, the last step of ethanol synthesis pathway is catalyzed by ethanol dehydrogenase. However, this step is reversible, and some ethanol dehydrogenase is more inclined to catalyze the decomposition of ethanol. In addition, acetaldehyde, the substrate of ethanol dehydrogenase, will also be catalyzed by endogenous acetaldehyde dehydrogenase to produce acetyl coA. In order to avoid the consumption of ethanol and acetaldehyde and improve the accumulation of ethanol in the fermentation broth, the inventor found the potential ethanol and acetaldehyde consumption of ethanol dehydrogenase and acetaldehyde dehydrogenase by analyzing the transcriptome data under ethanol stress, which were verified by experiments. The main genes identified were ethanol dehydrogenase gene Mtadh (Mycth_55576) and acetaldehyde dehydrogenase gene Mtaldh (Mycth_2140820). Therefore, this example knocks out these two genes.

1. Construction of sgRNA Expression Frame

The protopacer (eg. The target site) of target genes mtadh (Mycth_55576) and mtaldh (Mycth_2140820) was designed by software sgRNACas9 tool. The sequence sgRNA promoter, protopacer and sgRNA were connected by fusion PCR, and the sgRNA expression frame vector was constructed by gene overlap extension (SOE).

sgRNA expression plasmids U6p-mtadh-sgRNA and U6p-mtaldh-sgRNA were obtained by SOE-PCR amplification. The detailed construction process is shown in example 6. The sequences are shown in SEQ ID No.11 and SEQ ID No.13 respectively.

### 2. Amplification of Donor DNA

In the invention, the donor DNA fragment consists of about 700 bp homologous fragment upstream/downstream of the target gene and PtrpC-hygR fragment of hygromycin resistance gene expression frame respectively. The full-length linearized donor DNA fragments donor-mtadh and donor-mtaldh are obtained by fusion PCR, and their nucleic acid sequences are shown in SEQ ID No. 8 and SEQ ID No.10, respectively.

The PCR reaction system and reaction conditions in this example are the same as those described in Example 1.

The primers used for vector construction in this example are as follows:

| SEQ ID NO. | Primer | Sequence (5′-3′) |
|---|---|---|
| 131 | sgRNA-a | AGGATCGGTGGAGTGAAGTTCG |
| 132 | 55576sgRNA-b | CGGCGCTCTGGATGATCGAGGAAAGAAAGAAAAGAAG |
| 133 | 55576sgRNA-c | CATCCAGAGCGCCGGCAGTTTTAGAGCTAGAAATA |
| 134 | sgRNA-d | AAAAAAAGCACCGACTCGGTGCC |
| 135 | 55576donor-up-F | TCAAGTGTCATGATCTCGCGTG |
| 136 | 55576donor-up-R | tcatcttctgGGTTTGGATGAATGGATG |
| 137 | 55576donor-hygR-F | catccaaaccCAGAAGATGATATTGAAGGAG |
| 138 | 55576donor-hygR-R | cgcaacttgaGAAAGAAGGATTACCTCTAAAC |
| 139 | 55576donor-Down-F | tccttctttcTCAAGTTGCGGGGCGCGT |
| 140 | 55576donor-Down-R | AGGACTATTATCATCCTGGGGGA |
| 142 | 2140820sgRNA-b | GATGGGCGTCTTGAGCTCGAGGAAAGAAAGAAAAGAAG |
| 143 | 2140820sgRNA-c | GCTCAAGACGCCCATCACGTTTTAGAGCTAGAAATAGCA |
| 14 | 2140820donor-up-F | GTGCCGCTATCCAAGCATATTGC |
| 18 | 2140820donor-up-R | tcatcttctgTGGTTGTGGTGGTGGTGG |
| 21 | 2140820donor- | accacaaccaCAGAAGATGATATTGAAGGAG |
| | hygR-F | |
| 22 | 2140820donor- | gtcttcttctGAAAGAAGGATTACCTCTAAAC |
| | hygR-R | |
| 23 | 2140820donor- | tccttctttcAGAAGAAGACGTTCGAGGTC |
| | Down-F | |
| 24 | 2140820donor- | TCCATGTGCGACGAGAGGGCGGC |
| | Down-R | |

3. Determination of Ethanol Production Capacity of M. thermophila Transformants

Cas9 expression frame, sgRNA expression plasmid U6p-mtadh-sgRNA,U6p-mtaldh-sgRNA and donor DNA fragments donor-mtadh and donor-mtaldh were mixed in equal proportion and co-transformed into the protoplast cells of strain E5. Cas9, mediated by gRNA, identified the target site by pairing the target sequence with the DNA strand of the target gene on the host cell genome, and then homologous recombination occurred between the donor DNA fragment and the sequences on both sides of the target site. In order to achieve the purpose of editing the genome, the transformants were screened by adding hygromycin to the plate. The transformation method and verification method of introducing the target gene fragment into M. thermophila are the same as those described in Example 1.

The starting strain of this transformation is strain E5 (see example 6 for the construction process). The strain obtained by knocking out the ethanol dehydrogenase gene Mtadh (Mycth_55576) is named strain E14, and the strain obtained by knocking out the acetaldehyde dehydrogenase gene Mtaldh (Mycth_2140820) is named strain E15. Strains E14, E15 and E5 were inoculated into 75 g/L glucose and 75 g/L cellulose (Avicel) medium respectively (see example 1 for the formula). The transformed sporozoites were collected with distilled water and filtered with two layers of distilled mirror paper. The number of spores was calculated. The inoculation concentration was 2.5×10⁵/ mL, the volume of medium was 100 mL/bottle, cultured at 45° C. for 7 days, and the shaker speed was 150 rpm.

The supernatant of the sample was centrifuged and the ethanol content was determined. The results are shown in FIG. 11: when glucose was used as the carbon source, the ethanol yield of strain E14 and E15 were 18.84 g/L and 17.93 g/L respectively, which were higher than that of the starting strain E5 (15.96 g/L), 18.05% and 12.34% higher than that of E5 respectively. When fermenting with cellulose (Avicel) as carbon source for 7 days, the ethanol yield of strain E14 and E15 was 3.12 g/L and 3.59 g/L respectively, which was higher than that of starting strain E5 (2.568 g/L), which was 21.5% and 39.8% higher than that of E5, respectively. It shows that the knockout of endogenous ethanol dehydrogenase gene Mtadh and acetaldehyde dehydrogenase gene Mtaldh can reduce the metabolic consumption of ethanol by M. thermophila, and then improve the ethanol production of the strain under the conditions of glucose and cellulose (Avicel).

### Example 11

Firstly, the coding gene of phosphofructokinase2 (PFK2, Mycth_71484, Gene ID: 11510101) was knocked out in this example.

1. Construction of sgRNA Expression Frame

The protopacer (eg. The target site) of the target gene pfk2 (Mycth_71484) was designed by the software sgRNACas9 tool. The sgRNA promoter, protopacer and scaffold were connected by fusion PCR to construct the sgRNA expression frame U6p-pfk2-sgRNA. The sequence is shown in SEQ ID No.167. The specific operations were as follows: firstly, the promoter and protopacer DNA fragments were amplified with primers sgRNA-a and 71484SgRNA-b; protopacer and scaffold fragments were amplified with primers 71484SgRNA-cand sgRNA-d, and then the full length of sgRNA expression frame was amplified with primers sgRNA-a and sgRNA-d. The PCR reaction system and reaction conditions are the same as those in example 1.

### 2. Construction of Donor DNA Vector

In the invention, the donor DNA fragment is respectively composed of the upstream homologous arm of the target gene, the PtrpC-bar fragment of the glyphosate resistance gene expression frame and the downstream homologous arm, which are connected together by fusion PCR, and finally constructed into the donor DNA fragment donor pfk2. The nucleic acid sequence is shown in SEQ ID No.168. The construction process was as follows: firstly, the upstream homologous arm DNA was amplified with primers 71484up-Fand 71484up-R, the expression frame PtrpC-bar fragment of glufosinate resistance gene was amplified with primers 71484Bar-Fand 71484Bar-R, and the downstream homologous arm was amplified with 71484down-Fand 71484down-R. Then, the upstream homologous arm and PtrpC-bar fragment were used as templates at the same time. The fusion DNA fragment of the upstream homologous arm and the glyphosate resistance gene expression frame was amplified with primers 71484up-Fand 71484Bar-R, then the fusion DNA fragment and the downstream homologous arm were used as templates at the same time, and the full-length donor DNA fragment was amplified with primers 71484up-F and 71484down-R. Finally, the donor DNA sequence was proved to be correct by gene sequencing.

The primers used for vector construction in this example are as follows:

| | | |
|---|---|---|
| 183 | sgRNA-a | AGGATCGGTGGAGTGAAGTTCG |
| 184 | 71484SgRNA-b | ATACTGGTCTGCAATCTCCGAGGAAAGA |
| | | AAGAAAAGAAGA |
| 185 | 71484SgRNA-c | AGATTGCAGACCAGTATCGTTTTAGAGC |
| | | TAGAAATAGCAAGT |
| 186 | sgRNA-d | AAAAAAAGCACCGACTCGGTGCC |
| 187 | 71484up-F | tacacagtacacgaggacttctagaGGC |
| | | TCGGGTCGGTTAATG |
| 188 | 71484up-R | ccttcaatatCTTTGCTGAGCGACAGCC |
| 189 | 71484Bar-F | ctcagcaaagATATTGAAGGAGCACTTTTTGGG |
| 190 | 71484Bar-R | gaggcagatgTCAGATCTCGGTGACGGG |
| 191 | 71484down-F | cgagatctgaCATCTGCCTCACGGGGTG |
| 192 | 71484down-R | aatatcatcttctgtcgaggaattcTCA |
| | | ATGGCGTAAAGTTCAACATCAG |
| 193 | Pfk2m-YZ-F | CATCATCGATACACTGGTTCTTGACAA |
| 194 | Pfk2m-YZ-R | CTCAGGGTCCTGACCCTGGTAATCC |

### 3. Construction of Phosphofructokinase 2 Mutant

In this example, the mutation sites of phosphofructokinase 2 (PFK2, Mycth_71484) are H233G, E306G and H371G. The inactivation information of phosphokinase can be selected based on the structural mutation of phosphokinase 2, which can only retain the activity of phosphokinase 2. The nucleotide and amino acid sequences after mutation are shown in SEQ ID No. 170 and SEQ ID No.171, respectively. The mutant pfk2 gene was amplified by fusion PCR with the genomic DNA of M thermophila as the template. The specific operations are as follows: firstly, take genomic DNA as the template, and use 71484mut-a and 71484mut-b, 71484mut-c and 71484mut-d, 71484mut-e and 71484mut-f, 71484mut-g and 71484mut-h four pairs of primers to amplify the four fragments of the mutated pfk2 gene, and then use the first two fragments as the template at the same time, and 71484mut-a and 71484mut-d as the primers to amplify the fusion fragments of the first two DNA fragments. Similarly, use 71484mut-e and 71484mut-h as the primers, the fusion fragments of the latter two DNA fragments were amplified by using the latter two DNA fragments as templates at the same time. Finally, using the above two fused DNA fragments as templates, the full-length mutant pfk2 gene was amplified by primers 71484mut-a and 71484mut-h. Then, using the fused full-length mutant pfk2 motif template, the DNA fragment used to connect to the vector was amplified with primers Pfk2m-Fand Pfk2m-R. the fragment was recombined into the linearized vector pAN52-TB-Intron digested by BcuI and BamHI by Gibson assembly of NEB. The promoter of the vector was Ptef promoter (nucleic acid sequence is shown in SEQ ID No. 2), and the recombinant expression plasmid pAN52-tef-pfk2 of the mutant pfk2 gene was obtained.

The primers used for vector construction in this example are as follows:

| SEQ ID NO. | Primer | Sequence (5′-3′) |
|---|---|---|
| 173 | 71484mut-a | ATGGCTCCAAAGATTAACGGCA |
| 174 | 71484mut-b | TCAGACTCGCCGCCGCGAGACAGC |
| 175 | 71484mut-c | GCTGTCTCGCGGCGGCGAGTCTGA |
| 176 | 71484mut-d | CAGCGTCTAGGCCATCCAGCGCCT |
| 177 | 71484mut-e | AGGCGCTGGATGGCCTAGACGCTG |
| 178 | 71484mut-f | ATGACGGCCTGGCCCGAGATGATG |
| 179 | 71484mut-g | CATCATCTCGGGCCAGGCCGTCAT |
| 180 | 71484mut-h | TCAAATGCCGCCTTCTGGGGTTG |
| 181 | Pfk2m-F | gccagtttcgttatcagaactagt |
| | | ATGGCTCCAAAGATTAACGG |
| 182 | Pfk2m-R | gatttcagtaacgttaagtggatc |
| | | cTCAAATGCCGCCTTCTGG |

4. Transformation of M. thermophila

The DNA (donor-pfk2, Cas9 expression frame and U6p-pfk2-sgRNA) used for knockout was co transformed into wild-type strain ATCC42464 of M. thermophila, and the recombinant expression plasmid pAN52-tef-pfk2m linearized by HindIII was transformed into the wild-type strain ATCC42464 and the wild-type strain with knockout pfk2 gene, respectively. The transformation method and genome extraction method were the same as those in embodiment 1.

5. PCR Validation of M. thermophila Transformants

For the transformant with knockout pfk2 gene, the extracted genomic DNA of the transformants is used as the template, and the knockout transformants are verified by PCR with primers 71484up-F and 71484down-R. PCR products were subjected to 1% agarose gel electrophoresis (110V voltage, min). The gene amplification bands were observed under the gel imaging system. It was shown that under the guidance of primers 71484up-F and 71484down-R, the transformant obtained a target band of ˜1380 bp by PCR amplification, The wild-type strain obtained the target band of ˜2000 bp by PCR amplification (the length of donor DNA amplified by this primer was 1380 bp, and the length of DNA amplified by wild-type genome was about 2000 bp). The results showed that the phosphofructokinase 2 gene (pfk2, Mycth_71484) had been knocked out from the genome of M thermophila, and the knockout strain was named E17 strain.

Based on the knockout of pfk2 gene (strain E17), the transformant of the mutant phosphofructokinase 2 gene was further overexpressed. Taking the extracted genomic DNA of the transformant as a template, the transformant of the overexpressed mutant pfk2 was verified by PCR with primers pfk2m-yz-F and pfk2m-yz-R. The PCR products were subjected to 1% agarose gel electrophoresis (110V, 30 min). The gene amplification bands were observed under the gel imaging system. It was shown that the transformant obtained ˜592 bp of the target band by PCR amplification, while the starting strain E17 did not amplify any band by PCR amplification (only the strain with overexpression mutation pfk2 gene could amplify ˜592 bp of the target band under the above primers), these results showed that the fructose kinase gene had been transferred into the genome of M thermophila, and the strain was named strain E18.

In order to verify the effect of direct overexpression of the mutated phosphofructokinase 2 gene, the invention also overexpressed the mutated pfk2 gene in the wild-type strain, and PCR verified the transformant with primers pfk2m-yz-F and pfk2m-yz-R using the genomic DNA of the obtained transformant as the template. The PCR products were subjected to 1% agarose gel electrophoresis (110V, 30 min). The gene amplification bands were observed under the gel imaging system. It showed that the transformant obtained˜592 bp of the target band by PCR amplification, while the original strain WT did not amplify any bands by PCR amplification (only the strain with overexpression mutation pfk2 gene could amplify˜592 bp of the target band under the above primers), the results showed that the fructose kinase gene had been transferred into the genome of M. thermophila, and the strain was named strain E19.

### 6. Determination of Ethanol Production Capacity of Transformant

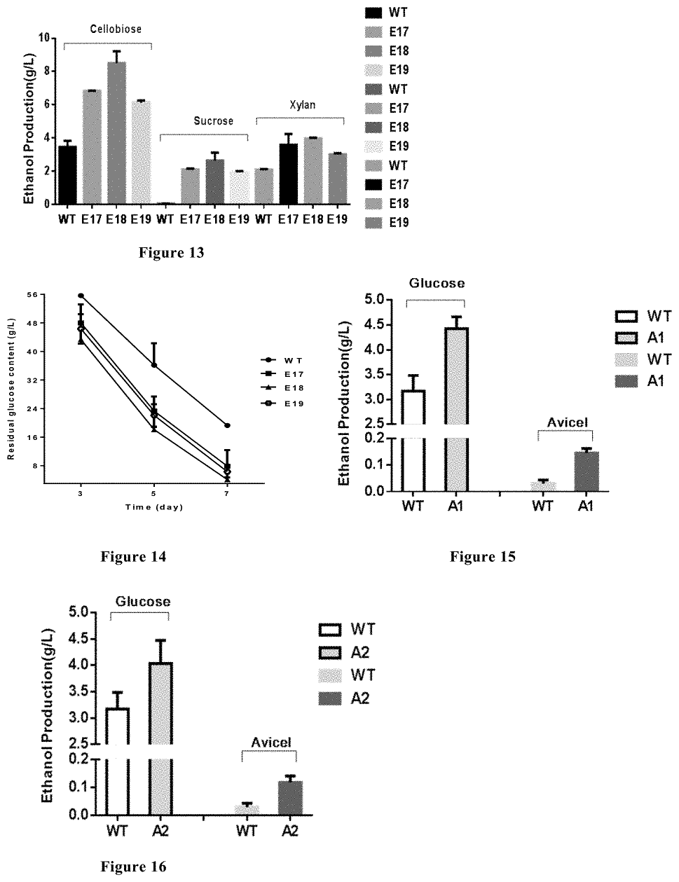

Wild-type strain ATCC42464 (WT), strain E17 with wild-type knockout pfk2 gene, strain E18 with E17 overexpressing mutated pfk2 gene and strain E19 with wild-type overexpressing mutated pfk2 gene were inoculated into 75 g/L glucose, 75 g/L cellulose, 75 g/L cellobiose, 75 g/L xylan and 75 g/L sucrose medium, respectively. After 7 days of culture, the samples were centrifuged, the supernatants were taken, and the contents of ethanol in the culture medium were measured. The ethanol yields are shown in FIG. 12 (glucose and cellulose conditions) and FIG. 13 (cellobiose, sucrose and xylan conditions): when glucose is used as carbon source, the ethanol yield of strain E17 is 4.724 g/L, which is 49.1% higher than that of the original strain WT (3.168 g/L); the ethanol yield of strain E18 was 7.371 g/L, which was 56.03% higher than that of strain E17; the ethanol yield of strain E19 was 5.839 g/L, which was 84.3% higher than that of the original strain WT (3.168 g/L). When fermenting with cellulose as carbon source for 7 days, the ethanol yield of strain E17 was 0.153 g/L, which was significantly higher than that of the original strain WT (0.0305 g/L), increased by 400%; the ethanol yield of strain E18 was 0.259 g/L, which was 69.5% higher than that of strain E17; the ethanol yield of strain E19 was 0.168 g/L, which was 449% higher than that of the original strain WT (0.0305 g/L). When cellobiose was used as carbon source, the ethanol yield of strain E17 was 6.826 g/L, which was 97.6% higher than that of the original strain WT (3.454 g/L); the ethanol yield of strain E18 was 8.504 g/L, which was 24.6% higher than that of strain E17; the ethanol yield of strain E19 was 6.12 lg/L, which was 77.2% higher than that of the original strain WT (3.454 g/L). When sucrose was used as carbon source, the ethanol yield of strain E17 was 2.0875 g/L, which was 51.8 times higher than that of the original strain WT (0.0395 g/L). The ethanol yield of strain E18 was 2.633 g/L, which was 26.1% higher than that of strain E17; the ethanol yield of strain E19 was 1.924 g/L, which was 47.7 times higher than that of the original strain WT (0.0395 g/L). When xylan was used as carbon source, the ethanol yield of strain E17 was 3.581 g/L, which was 72.4% higher than that of the original strain WT (2.077 g/L). The ethanol yield of strain E18 was 3.962 g/L, which was 10.6% higher than that of strain E17; the ethanol yield of strain E19 was 3.002 g/L, which was 44.5% higher than that of the original strain WT (2.077 g/L). It shows that knockout of pfk2 gene and overexpression of mutatedpfk2 gene can effectively improve the ethanol production of M thermophila under the conditions of glucose, cellulose, cellobiose, sucrose and xylan, that is, it is universal under the conditions of different carbon sources as substrates.

In addition, the glucose metabolism rates of the above four strains were also compared. The four strains were inoculated in the medium with 75 g/L glucose as carbon source at the same time (the formula is the same as that in example 4). Samples were taken at the same time on the 3rd, 5th and 7th days after inoculation to determine the residual glucose content in the medium. The results are shown in FIG. 14: at the above time points, the residual glucose content in the medium of strain E18 is less than that of strain E17; the content of residual glucose in the medium of strain E17 was less than that of wild-type strain WT; the content of residual glucose in the medium of strain E19 was also less than that of wild-type strain WT. These results showed that strain E18 metabolized glucose faster than strain E17, strain E17 metabolized glucose faster than strain WT, and strain E19 metabolized glucose faster than strain WT. It shows that knockout of pfk2 gene and overexpression of mutatedpfk2 gene can accelerate the metabolic rate of glucose.

### Example 12

1. Construction of adhE Expression Vector (pAN52-adhE)

Taking the acetaldehyde dehydrogenase adhE (amino acid sequence as shown in SEQ ID No.195) of Thermoanaerobacterium saccharolyticum as the template, the gene was synthesized by gene synthesis method through codon optimization, and the DNA sequence is shown in SEQ ID No.196. Using synthetic adhE gene as template, adhE gene was amplified by primers adhE-F and adhE-R; The mitochondrial localization signal sequence (nucleotide sequence as shown in SEQ ID No.197 and amino acid sequence as shown in SEQ ID No.198) of cis aconitase (Mycth_2309261) was amplified with primers 9261MTS-Fand 9261MTS-R using the genome of M. thermophila (ATCC42464) as the template, and then the fusion fragments of the two DNA were amplified with primers 9261MTS-Fand adhE-R. Then, the fusion fragment was recombined into the linearized vector pAN52-TB-Intron (Liu Q, Li J, Ying S, Wang J, Sun W, Tian C, Feng M. 2014. Unveiling equal importance of two 14-3-3 proteins for morphogenesis, conidiation, stress tolerance and virulence of an insect pathogen. Environ Microbiol. doi: 10.1111/1462-2920.12634) by Gibson assembly of NEB, The recombinant expression plasmid pAN52-adhE of adhE gene was obtained.

The PCR reaction system and reaction conditions were as Example 1. The primers used for vector construction are as follows:

| SEQ ID NO. | Primer | Sequence (5′-3′) |
|---|---|---|
| 141 | adhE-F | ccggcgcatgATGGCCACCACCAAGACC |
| 158 | adhE-R | gatttcagtaacgttaagtggatccTCAGGCGCCGTAGGCCTT |
| 161 | 9261MTS-F | ccacatcacagaaatcaaaactagtATGTTGGCTTCTCGTCAGC |
| 169 | 9261MTS-R | tcttggtggtggccatCATGCGCCGGAGACCAAG |
| 172 | adhE-YZ-F | CTGTCGGCAGCGCGAGCCTTGGTCTCCG |
| 199 | adhE-YZ-R | GAAGGCGTGTTGGTGTTGTAGATGTCG |

2. Construction of Adh1 Overexpression Vector (pAN52-adh1)

Using the genomic DNA of S. cerevisiae S288C (taxlD: 559292) as the template, adh1 gene was amplified with primers adh1-F and adh1-R (Gene ID: 854068). The mitochondrial localization signal sequence (nucleotide sequence as shown in SEQ ID No.200) of mitochondrial malate dehydrogenase (Mycth_2305575) was amplified with primers 5575MTS-Fand 5575MTS-R using the genome of M thermophila (ATCC42464) as a template, and then the fusion fragments of the two DNA were amplified with primers 5575MTS-Fand adh1-R. Then, the fusion fragment was recombined into the linearized vector pAN52-TB-Intron (Environ Microbiol. 015.17(4):1444-62) digested by BcuI and BamHI by Gibson assembly of NEB, and the recombinant expression plasmid pAN52-adh1 of adh1 gene was obtained.

The PCR reaction system and reaction conditions were as Example 1. The primers used for vector construction are as follows:

| SEQ ID NO. | Primer | Sequence (5′-3′) |
|---|---|---|
| 47 | 5575MTS-F | ccacatcacagaaatcaaaactagtATGTTCCTGGGCAGCCGC |
| 48 | 5575MTS-R | tttctgggatagacatACCGAGAACGGCGACCTTG |
| 49 | adh1-F | cgttctcggtATGTCTATCCCAGAAACTC |
| 50 | adh1-R | tgttaataacccacgcgtgggcggatccTTATTTAGAAGTGTCAACAACG |
| 53 | adh1-YZ-F | CAGCCGCATCCAGACCCGCGCCTTCTC |
| 72 | adh1-YZ-R | TTATTTAGAAGTGTCAACAACGTATCTACCA |

3. Construction of Pdc1 Overexpression Vector (pAN52-Pdc1)

Using the genomic DNA of S. cerevisiae S288C (taxlD: 559292) as the template, the pdc1 gene (Gene ID: 850733, nucleotide sequence shown in SEQ ID No.214 and amino acid sequence shown in SEQ ID No.215) was amplified with primers pdc1-Fand pdc1-R. The mitochondrial localization signal sequence of cis-aconitase (Mycth_2309261) was amplified by primers MTS-pdc1-Fand MTS-pdc1-R with the genome of M. thermophila (ATCC42464) as the template (nucleotide sequence is shown in SEQ ID No.197). Then, using the two amplified DNA fragments as the template, the fusion fragments of the two DNA were amplified by primers MTS-pdc1-Fand pdc1-R. Then, the fusion fragment was recombined into the linearized vector pAN52-TB-Intron (Environ Microbiol. 015.17(4):1444-62) digested by BcuI and BamHI by Gibson assembly of NEB, and the recombinant expression plasmid pAN52- pdc1 of pdc/gene was obtained.

The PCR reaction and reaction conditions were as Example 1. The primers used for vector construction are as follows:

| SEQ ID NO. | Primer | Sequence (5′-3′) |
|---|---|---|
| 73 | pdc1-F | ccggcgcatgATGTCTGAAATTACTTTGGG |
| 74 | pdc1-R | gatttcagtaacgttaagtggatccTTATTGCTTAGCGTTGGTAG |
| 75 | MTS-pdc1-F | ccacatcacagaaatcaaaactagtATGTTGGCTTCTCGTCAGC |
| 108 | MTS-pdc1-R | tttcagacatCATGCGCCGGAGACCAAG |
| 109 | pdc1-YZ-F | ATGTCTGAAATTACTTTGGGTAAA |

The transformation method and verification method of subsequent target gene overexpression vector into M. thermophila are the same as those described in Example 1.

4. PCR Validation of M. thermophila Transformants

The transformants obtained by transforming pAN52-adhE and pAN52-adh1 vectors at the same time, took the extracted transformant genomic DNA as template, adhE-YZ-F and adhE-YZ-R as primers, and verified whether adhE gene was integrated into the genome by PCR amplification; adh1-YZ-Fand adh1-YZ-R were used as primers to verify whether Adh1 gene was integrated into the genome. The obtained strain transferred into two expression vectors, pAN52-adhE and pAN52-adh1, was named strain A1.

Similarly, for the transformants obtained by simultaneous transformation of pAN52-pdc1 and pAN52-adh1 vectors, the extracted genomic DNA was used as template, pdc1-YZ-Fand pdc1-YZ-R were used as primers, and the integration of pdc1 gene into the genome was verified by PCR; adh1-YZ-F and adh1-YZ-R were used as primers to verify whether adh1 gene was integrated into the genome. The obtained strains transferred into both pAN52-pdc1 and pAN52-adh1 vectors were named as strain A2.

5. Determination of Ethanol Production Capacity of Transformants of M. thermophila

Strains A1 and A2 and wild-type ATCC42464 of M. thermophila verified by the above PCR were inoculated into 75 g/L glucose and 75 g/L cellulose (Avicel) medium respectively (see example 1 for the formula). The transformed sporozoites collected with distilled water were filtered by two layers of distilled mirror paper, and the number of spores was calculated. The inoculation amount was 2.5×105 spores/mL, the medium volume was 100 mL/bottle, cultured under light, 45° C., and the rotating speed of the shaker was 150 rpm. The ethanol content was measured on the 7th day.

The ethanol content of the treated samples was determined by high performance liquid chromatography, in which the detector was differential detector, 5 mM H₂SO₄ was mobile phase, and the flow rate was 0.5 mL/min. The ethanol yield of A1 transformant transferred into pAN52-adhE and pAN52-adh1 vectors is shown in FIG. 15. When glucose is used as carbon source for fermentation for 7 days, the ethanol yield of strain A1 is 4.42 g/1, which is 39.4% higher than that of wild-type strain (M. thermophila ATCC42464, 3.17 g/1). When fermenting with cellulose (avive1) as carbon source for 7 days, the ethanol yield of strain A1 was 145 mg/L, which was 3.75 times higher than that of wild-type strain (M. thermophila ATCC42464, 30.5 mg/L). The results showed that the expression of adhE and adh1 genes in mitochondria could improve the ethanol production of M thermophila when glucose and cellulose (Avicel) were used as carbon sources.

The ethanol yield of A2 transformant transferred into pAN52-pdc1 and pAN52-adh1 vectors is shown in FIG. 16. When glucose is used as carbon source for fermentation for 7 days, the ethanol yield of A2 strain is 4.03 g/L, which is 27% higher than that of wild-type strain (M. thermophila ATCC42464, 3.17 g/L). When fermenting with cellulose (avive1) as carbon source for 7 days, the ethanol yield of strain A2 was 118 mg/L, which was 2.87 times higher than that of wild-type strain (M. thermophila ATCC42464, 30.5 mg/L). The results showed that the expression of pdc1 and adh1 genes in mitochondria could improve the ethanol production of M. thermophila with glucose and cellulose (Avicel) as carbon sources.

1. A construction method of genetic engineering fungi of filamentous fungi, which is characterized in that the filamentous fungi overexpress the positive regulation genes of ethanol synthesis, and/or down regulate the expression of the negative regulation genes of endogenous ethanol synthesis to obtain genetic engineering fungi, compared with the original strain, the ethanol synthesis ability of the genetically engineered strains are improved.

2. The construction method as claim 1, which is characterized in that the filamentous fungiare cellulose degrading filamentous fungi; or, the filamentous fungi are selected from Neurospora, Aspergillus, Trichoderma, Penicillium, Myceliophthora, Sporotrichum, Fusarium, Rhizopus Mucor and Paecilomyces; more preferably or, the Myceliophthora fungi are selected from Myceliophthora thermophila and M. heterothalica.

3. The construction method as claim 1, which is characterized in that the positive regulatory genes of ethanol synthesis are selected from the ethanol synthesis genes with strengthening the ethanol synthesis pathway, improving the sugar transport capacity, accelerating the glycolysis rate, improving cytoplasmic reducing power and containing mitochondrial localization signal sequence; and negative regulation genes of ethanol synthesis are selected from the genes in the branch pathway of ethanol synthesis, the shuttle pathway of cytoplasmic reducing force to mitochondria, the endogenous ethanol metabolism pathway and the electron transfer chain (respiratory chain).

4. The construction method as claim 1, which is characterized in that the genetically engineered fungi with the shuttle of cytoplasmic reducing force to mitochondria is reduced or blocked, and/or the intensity of respiratory chain is weakened, and/or the by-product pathway of ethanol synthesis is weakened, and/or the ethanol synthesis pathway is strengthened, and/or the transport of sugar molecules is strengthened, and/or the glycolysis rate is accelerated.

5. The construction method as claim 1, which is characterized in that the overexpression of the positive regulation genes of ethanol synthesis are realized through the introduction of exogenous and/or endogenous positive regulation genes of ethanol synthesis, wherein the positive regulation genes of ethanol synthesis are selected from one or more of ethanol dehydrogenase, glucose transporter, cellobiose transporter and pyruvate decarboxylase.

6. The construction method as claim 1, which is characterized in that the introduction is to transfer the expression vector carrying the positive regulation genes of exogenous or endogenous ethanol synthesis into the filamentous fungi, and the preferred promoters are tef, gpdA, trpC, cbhl and glaA promoter; and the imported positive regulatory genes of exogenous ethanol synthesis come from Saccharomyces cerevisiae and/or Zymomonas mobilis.

7. The construction method as claim 1, which is characterized in that one of the ethanol dehydrogenase coding geneScadh1, pyruvate decarboxylase gene Scpdc1, glucose transporter coding gene glt-1, cellobiose transporter coding gene cdt-1/cdt-2 or a combination thereof is overexpressed in the filamentous fungus; or, the overexpression of the positive regulatory genes of ethanol synthesis in the filamentous fungi are selected from ethanol dehydrogenase and pyruvate decarboxylase; and the preferred ethanol dehydrogenases are ethanol dehydrogenase ScADH1 from Saccharomyces cerevisiae and ZmADH1 from Zymomonas mobilis, and the preferred pyruvate decarboxylases are pyruvate decarboxylases ScPDC1 and ScPDC5 from Saccharomyces cerevisiae and ZmPDC from Zymomonas mobilis.

8. The construction method as claim 1, which is characterized in that the down-regulated gene expression is to inactivate or reduce the expression or decrease the activity of the negative regulation genes of endogenous ethanol synthesis through gene knockout or small RNA interference or gene editing or replacement of promoter or gene mutation; or, the gene editing is a genome editing method based on CRISPR/Cas9.

9. The construction method as claim 8, which is characterized in that the negative regulation genes of endogenous ethanol synthesis in the strain are selected from one or more combination genes of lactate dehydrogenase, 1-phosphate mannitol dehydrogenase, cytoplasmic malate dehydrogenase, glycerol-3-phosphate dehydrogenase, external NADH dehydrogenase, ethanol dehydrogenase, acetaldehyde dehydrogenase and phosphofructokinase 2 genes, and the expression level is reduced or lost, and/or reduce the expression of cytochrome C oxidase, a negative regulation gene of endogenous ethanol synthesis in the strain; or, the endogenous ethanol synthesis negative regulatory genes are selected from the cytoplasmic malate dehydrogenase, glycerol-3-phosphate dehydrogenase; or, the endogenous ethanol synthesis negative regulatory genes are cytochrome C oxidase coding gene and/or phosphofructokinase 2 gene.

10. The construction method as claim 1, which is characterized in that the filamentous fungi overexpress the cellobiose transporter coding genes cdt-1 and/or cdt-2 of Neurospora crassa, and down regulate the expression of lactate dehydrogenase genes ldh-1 and/or ldh-2; or
the ethanol dehydrogenase of S. cerevisiae is overexpressed in the filamentous fungus and the expression of external NADH dehydrogenase is down regulated, wherein one or both of the external NADH dehydrogenase genes nde1 and nde2 are down regulated at the same time; or
the ethanol dehydrogenase of S. cerevisiae is overexpressed in the filamentous fungus and the expression of cytochrome C oxidase gene is down regulated.

11. (canceled)

12. (canceled)

13. The construction method as claim 5, which is characterized in that the ethanol dehydrogenase of S. cerevisiae is overexpressed in the filamentous fungus, the expression of cytoplasmic malate dehydrogenase Mdh is down regulated/knocked out, and the expression of glycerol-3-phosphate dehydrogenase gene gpd is optionally further down regulated /knocked out.

14. The construction method as claim 5, which is characterized in that the filamentous fungi overexpressing the ethanol dehydrogenaseScADH1 is from S. cerevisiae and ZmADH1 is from Zymomonas mobilis, overexpression of pyruvate decarboxylase is the pyruvate decarboxylase ScPDC1 and ScPDC5 from Saccharomyces cerevisiae and ZmPDC from Zymomonas mobilis; or, further overexpress glucose transporter coding gene glt-1, and down-regulate cytoplasmic malate dehydrogenase Mdh, down regulate/knockout ethanol dehydrogenase gene Mtadh, and down regulate/knockout acetaldehyde dehydrogenase gene Mtadh.

15. The construction method as claim 5, which is characterized in that the filamentous fungi overexpress the ethanol dehydrogenase and pyruvate decarboxylase gene pdc1 from Saccharomyces cerevisiae or Zymomonas mobilis, or further overexpress the glucose transporter coding gene glt-1, and further regulate the lactate dehydrogenase gene and 1-phosphate mannitol dehydrogenase.

16. The construction method as claim 5, which is characterized in that the filamentous fungi overexpress Saccharomyces cerevisiae ethanol dehydrogenase ScADH1, and/or Zymomonas mobilis derived ZmADH1, and/or overexpress Saccharomyces cerevisiae derived pyruvate decarboxylases ScPDC1, ScPDC5, and/or Zymomonas mobilis derived pyruvate decarboxylase ZmPDC.

17. The construction method as claim 9, which is characterized in that the endogenous phosphofructokinase 2 gene is knocked out in the filamentous fungus, and the mutant phosphofructokinase 2 gene is further expressed, wherein the mutant phosphofructokinase 2 refers to the loss or reduction of phosphatase activity due to the retention of kinase activity after mutation; and phosphofructose kinase 2 refers to the gene encoding the synthesis and degradation of fructose-2,6-diphosphate or its homologous gene; and the mutated phosphofructose kinase 2 refers to the corresponding amino acid sequence as gene ID: 11510101 which has one or two or three mutation sites in H233, E306 and H371 to reduce or lose phosphatase activity.

18. The construction method as claim 5, which is characterized in that overexpression of ethanol synthesis gene containing mitochondrial localization signal sequence in the filamentous fungus for ethanol synthesis is overexpression of acetaldehyde dehydrogenase and ethanol dehydrogenase genes containing mitochondrial localization signal sequence, or overexpression of pyruvate decarboxylase and ethanol dehydrogenase genes containing mitochondrial localization signal sequence, or overexpression of acetaldehyde dehydrogenase, ethanol dehydrogenase and pyruvate decarboxylase genes containing mitochondrial localization signal sequences.

19. The construction method as claim 18, which is characterized in that the pyruvate decarboxylase and ethanol dehydrogenase are derived from S. cerevisiae, and/or Zymomonas mobilis. The acetaldehyde dehydrogenase is derived from Thermoanaerobacterium saccharolyticum.

20. The construction method as claim 18, which is characterized in that the mitochondrial localization signal sequence refers to the N-terminal amino acid sequence used to guide the transmembrane transfer of protein to the mitochondria from the localization signal sequence of mitochondrial matrix protein of M. thermophila.

21. (canceled)

22. (canceled)

23. (canceled)

24. A method for producing ethanol, which is characterized in that the genetically engineered fungi obtained by the construction method according to claim 1 are cultured in a culture medium containing monosaccharide or/and glycan, and ethanol is collected from the culture.

25. The method for producing ethanol as claim 24, which is characterized in that the monosaccharide is glucose, xylose, arabinose or a combination thereof; the glycan includes cellobiose, xylobiose, sucrose, maltose, xylooligosaccharide, cellooligosaccharide, cellulose, crystalline cellulose, hemicellulose, starch, plant biomass or a combination thereof; or, the plant biomass is selected from crop straw, forestry waste, energy plants or some or all of their decomposition products; or, the crop straw is selected from corn straw, wheat straw, rice straw, sorghum straw, soybean straw, cotton straw, bagasse and corncob; and the forestry waste is selected from branches and leaves and sawdust; the energy plant is selected from sweet sorghum, switchgrass, miscanthus, reed or a combination thereof.

26. The method for producing ethanol according to claim 24,
which is characterized in that the filamentous fungus is Myceliophthora or Trichoderma; or, the Myceliophthora is selected from the group consisting of M. thermophila and M. heterothallica. It is most preferred that the Myceliophthora fungus is selected from M. thermophila; or, the fermentation temperature is 40-60° C., or 45-52° C., or 48-50° C.


SEQUENCE LISTING (200 sequences)

SEQ ID NO: 1 - DNA, 1439 bp, Myceliophthora thermophila
  PeIF fragment of the promoter of eIF-5A gene
ccacatcatgtaaacagaggctcagaagctcctgccacactgggagggtttcgaagtctgacgactgccaatggaccccagccatcgcgagcacacagcagttcgcacgctcccattgggttcctcatcacgcagtcgctctccccgccaaccagcgccaggtccgggaacagcggcgcaaatgcgtatttgagggcgcctcgctcgagcaacctgtgcctgaccttctcctcctccttctgcaccttgcatctcgtcgcgtccactcgcaggcaaccacacatcctcctcctctcccaaaacccccccgctttttctttcccttgttggaattcgattgaaaaagaagacgggtccgtctagagaccgccttctcacctttctctcgacttctttctaggaaaagaagcaagagtcattcttcttgtccaccttctggttcacggaaggtcgaggagaagattgcctctgcccccaaagtcgccaacctggactttgaagcacgtgttccggtccctttcagtgtcttcccgtcctcgtacagggagtccgagaccgccacccaaacccactcccacgaagaggttgagatcaagctcccccagctcgccggacgggaaggtcaacactcttcattccaagcccaagcacatcttcctcccagcggagagggtcgcttcagagaagaagaggtccgcatcactcgtcaagaggaacatcaccgccgtcccggcatccgtgaagagttcgttcaccgcgaggagcgtcaccggtaagtttagtttttgttttgattcaccacccattgtcttccccgcctttttctttttcttcccttgctctcttgcccctgtctagtgtagggcattgccaaggccatcttcacacacacacacccccccccccaccctcagctgggggggggggggtggcctgggttgaccaagggacggtgaagactactactacttgagccactcaaacccatgcatgacacagggttttcctttttcttttctcttttcctttaactaaccaaccactccaacattagccctcagtcaacctactccgagtctcgcatcgagttcgatactgagcaccgcactcacaactccgtcattgacgttgctgagagcgagtatcgtgcccgtgtccagcccaactaccgcaaggaagcttccgtagtcggtaccaccgtcgacggatcccgcttcagccacagccgcaaggccagcagcaccacctccacccacaccgacgagtacaccgtcgatccccctagccaccgccccgtctacaagaaggagtcggttgaagtcgccggtaccactgttgacccccctgctcctcgttcgacctaccacgagcaggtgaacattgttgaagagaccgttgacgctcaccgttacgctcctcaacacaacaacaacaacaag

SEQ ID NO: 2 - DNA, 1216 bp, Myceliophthora thermophila
  Ptef promoter
catgtaccttgacgtcctccgaggttcgacatcagggttcgtcatagggagtgaaacacccgccatgattccgtagccgcgcgcgaagatacgaagcagatatttcacggacatggcggagatacttgtttcccgtactaaggtagtcatgtcggagacatctgaacgacagagctggccaagagaaccgaccagttgccccaggacgatctagacaaaaaaaaagagagatgagtgggccacttttgccacaacatcgacggccctgcgaccgcccccaggcaaacaaacaaaccgccgaacaataatacttttgtcattttaggaggagcgttgtatggataaaaacaacatctcgttgctgcagaatgtggacttcaaacttgcagaaaatgggaggcggatttgcatgatcggagggtagttgactcacgccgcaggctgcaaatccgtcctccattattccatgaacaacttcgtaaggttgggctgagcgccaatgcctaacggaccgggggccacagcgcaacgtcccacttaaaggccagcgtgacatgccagttccataccaagtagtggcaccagaggcggccaatgctcagtaagggcagggagggaggctcaaacgattggcaaaaagaggggcttgccagttcagttccctgtgcgagcgcgagaggggcagtttcaaatctggaggggtgtgttgcgctggtctgaagagaaagagaagactgtacttaataattgttcaaagagtccatcatcgcgttgcggactcctctagctgtatttagagccctatcattacttgtcgggtgcgaatcaaaataccgggatgcagccctctggcgatttgcatgcggttgtggaggaagtgaagcctgaatcgcggggctgggcggcaaagcacgacgtgaaattcctggcgaaattcgagggcttgccccaccgtggttgaagtttttgtgctgcgtaaccccaccaacccgccttgcccctcccgcctgcccataaaaacttcgacccctcctcaaatcttcttcgattcttcctcttcacttccttcgtcggcatacctgattcaagcaatcacctgccactttcaagtgcgtataccatcatcgatacactggttcttgacaagtacatcgtctctaactttcctttttgcagttttcattaagcgcaagtcgccagtttcgttcttcagaac

SEQ ID NO: 3 - DNA, 44 bp, Artificial sequence
  Primer
tctgcagatctttaattaactcgagccacatcatgtaaacagag

SEQ ID NO: 4 - DNA, 28 bp, Artificial sequence
  Primer
ggatagacatcttgttgttgttgttgtg

SEQ ID NO: 5 - DNA, 29 bp, Artificial sequence
  Primer
caacaacaagatgtctatcccagaaactc

SEQ ID NO: 6 - DNA, 47 bp, Artificial sequence
  Primer
gatttcagtaacgttaagtggatccttatttagaagtgtcaacaacg

SEQ ID NO: 7 - DNA, 359 bp, Myceliophthora thermophila
  Terminator
acgaacctctctgaaggaggttctgagacacgcgcgattcttctgtatatagttttatttttcactctggagtgcttcgctccaccagtacataaaccttttttttcacgtaacaaaatggcttcttttcagaccatgtgaaccatcttgatgccttgacctcttcagttctcactttaacgtagttcgcgtttgtctgtatgtcccagttgcatgtagttgagataaatacccctggaagtgggtctgggcctttgtgggacggagccctctttctgtggtctggagagcccgctctctaccgcctaccttcttaccacagtacactactcacacattgctgaactgacccatcatac

SEQ ID NO: 8 - DNA, 3311 bp, Myceliophthora thermophila
  donor DNA fragments donor-mtadh
tcaagtgtcatgatctcgcgtgtcatccactttcatcagcgttgacctggcccgcactccaggaaccccaccctatctgtgcctacataacataagtcgtccccaaacctgtcggcgttaagactccactattaccagaactgcccaaaattcttcatcaactagccccgaaaacctcggattaccgagtcgtggcattcttctaggagtgtgagataaggagaattcaagtcctcggaataattatgactgcgtgaatatttgttgtcgaagaactatcgtctccaccattgttgaagacgacccacattccaccgctttccattgtcaacaatgacgtcttgtccctgaccttaagcggcttattcgagaggggcccatagctgccgagcaggtagaagcgggacaatgaaggtatatatttctgaagagagacgtctgtaacacgcgagcaccaaagactctcatcaacacatcacaacttctgaacttgttccacgttgtcatcctgtccacttccctctaatagttcgcaccacgccagcaattataggatacaaacacgcttaccacagtacatccattcatccaaacccagaagatgatattgaaggagcatttttgggcttggctggagctagtggaggtcaacacatcaatgctattttggtttagtcgtccaggcggatcacaaaatttgtgtcgtttgacaagatggttcatttaggcaactggtcagatcagcccacttgtaagcagtagcggcggcgctcgaagtgtgactcttattagcagacaggaacgaggacattattatcatctgctgcttggtgcacgataacttgtgcgtttgtcaagcaaggtaagtgaacgacccggtcataccttcttaagttcgcccttcctccctttatttcagattcaatctgacttacctattctaccgaagcatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttcaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagccctgggttcgcaaagataattgcatgtttcttccttgaactctcaagcctacaggacacacattcatcgtaggtataaacctcgaaatcattcctactaagatggtatacaatagtaaccatggttgcctagtgaatgctccgtaacacccaatacgccggccgaaacttttttacaactctcctatgagtcgtttacccagaatgcacaggtacacttgtttagaggtaatccttctttctcaagttgcggggcgcgtcgctcaactatcgcgacttgttgattcccaaggtaacttggctacgttgccctccgcgacgtcccttctcattccgtgaccccaaccaagatatacacacacacacacacatgataatacgaaccagcagagttctaaccttccctcgtccaacttctagggtcaataccccttcccgctcaacctccccgtcgtcgcgtgctccgacggcgccggcgaggtggtggcggtcgggccccgcgtgaccaagtggaagcccggcgatcgtgtggtgacgctcttcaaccagggccaccagtcgggccccctcgacgaggccgccagcaagacgggcctgggcgggtgcatcgacggcacgctgcggcagtacggcgccttcgaccaggacggcctcgtccgcatgcccgcgaacctcgactttgtcgaggccgccaccctcacctgcgccgcgctgaccagctggaacgccctctacggcctgaggaggctgcagaagggtgagacggtgctcgtgcagggcacgggaggtgttagcttgttgcgctgcaggtgtgtattatcccccaggatgataatagtcct

SEQ ID NO: 9 - DNA, 567 bp, Myceliophthora thermophila
  sgRNA expression plasmids U6p-ldh2-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcgctgctccttgataggttaggttttagagctagaaatagcaagttaaaataaggctagtccgttatcaacttgaaaaagtggcaccgagtcggtgcttttttt

SEQ ID NO: 10 - DNA, 3672 bp, Myceliophthora thermophila
  donor DNA fragments donor-mtadh
gtgccgctatccaagcatattgcggggaagccgggggtcgcatccgcaaatcagaggaagaatctggccatggggcgcggacgagtgggactcccccgcagcatcggaggtttggagagaaagtggagggtgacccattacaaatgtatgtacaacgtatctccgtatgtacggactacatacctatgtccactctatggatctagtaccccatagtagtaggctgagagaagccccgaaaaagtcgcgacaggctggcttctgttttctttcttccagcgtgctggtctcctgatgaacgtcgtcgagacagaaagattcaagatggacaagtgggaaaacgcaataacggcaatgggcatgcgcgcctctggtgcttctcgagtgttcgttgagaggccgacgtcacttccattaatccgttgaggcgtcgaccccgcttggctgccgcctagcttggctatcatcgtgttccactcggccgtggcggggaggggtgaacggggggaggagtcggggcagcagcgatgcgatcgccgtgcttgcattccttgtctcgactgcattgccacacccacccggtcgtctccatacccctcaaccccatctcaactgttcaggaattcatcaatcgtttataagttcaggccaaacccttcatcccatctctttcctcctgctcctgcacctcatcctcccaagtcgtctcattcttctcaaatttaattccctcgattcgcactcctaaaggcgttcttcccgcgtacacagaggaagctctgagtgaacctccgctagctccagcaccacatccgccacggctgatctcctcctcctcttccaccaccaccacaaccacagaagatgatattgaaggagcatttttgggcttggctggagctagtggaggtcaacacatcaatgctattttggtttagtcgtccaggcggatcacaaaatttgtgtcgtttgacaagatggttcatttaggcaactggtcagatcagcccacttgtaagcagtagcggcggcgctcgaagtgtgactcttattagcagacaggaacgaggacattattatcatctgctgcttggtgcacgataacttgtgcgtttgtcaagcaaggtaagtgaacgacccggtcataccttcttaagttcgcccttcctccctttatttcagattcaatctgacttacctattctaccgaagcatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttcaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagccctgggttcgcaaagataattgcatgtttcttccttgaactctcaagcctacaggacacacattcatcgtaggtataaacctcgaaatcattcctactaagatggtatacaatagtaaccatggttgcctagtgaatgctccgtaacacccaatacgccggccgaaacttttttacaactctcctatgagtcgtttacccagaatgcacaggtacacttgtttagaggtaatccttctttcagaagaagacgttcgaggtcattaacccgagcaccgaagaggtcatctgctcggtgcacgaggccacagagaaggatgtcgacatcgccgtcgccgccgcccgcaaggcgtttgagggcgagtggcgccgcgtcaccccgcagcagcgcggcgtcctgctgctaaagctggccgatctcgccgagaagaacctggaccttctggccgcggtcgagtcgctcgacaacggcaagtccatcaccatggcccgcggcgacgtcgccgccgttgtcgggtgcatccgctactacggcggctgggccgacaagatcgagggcaggaccatcgacatcaacccggacatgttcagctacacgaggcgcgagccggtatgcgattcctttcacctctctgctgtgctcccctaccctactccgagagtacaagctaaccttggactttttgttccccgccctccggtagattggtgtctgcggccaaatcattccctggaacttcccgctgctcatgcttgcgtggaagatcggccctgcgctggccaccggaaacacggttgtcctcaagacggccgagcagaccccgctgtcggccctcgtctttgcccagttcgtcaaggaggccggcttccccccgggcgtcctcaacatcatctcgggtttcggcaggaccgccggcgccgccctctcgtcgcacatgga

SEQ ID NO: 11 - DNA, 567 bp, Myceliophthora thermophila
  sgRNA expression plasmids U6p-mtadh-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcgatcatccagagcgccggcagttttagagctagaaatagcaagttaaaataaggctagtccgttatcaacttgaaaaagtggcaccgagtcggtgcttttttt

SEQ ID NO: 12 - DNA, 567 bp, Myceliophthora thermophila
  sgRNA expression plasmids U6p-ldh1-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcgcacctttctggactcggtggttttagagctagaaatagcaagttaaaataaggctagtccgttatcaacttgaaaaagtggcaccgagtcggtgcttttttt

SEQ ID NO: 13 - DNA, 567 bp, Myceliophthora thermophila
  sgRNA expression plasmids U6p-mtaldh-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcgagctcaagacgcccatcacgttttagagctagaaatagcaagttaaaataaggctagtccgttatcaacttgaaaaagtggcaccgagtcggtgcttttttt

SEQ ID NO: 14 - DNA, 23 bp, Artificial sequence
  Primer
gtgccgctatccaagcatattgc

SEQ ID NO: 15 - DNA, 1372 bp, Myceliophthora thermophila
  The  fragment upstream of the glycosylaldehyde- 3-phosphate dehydrogenase coding gene gpdA of M. thermophila
ccgtcgacacgtgtgtcatccttccattgcaagcgatcactcgcagggcgtgacgatgaacgagatttttgcccggaccgattcgcggatatagcggcagccgaccagccctaccacactgatggccgtgtccctagtgtatgctcccagaaccgcaagcatacactgggcaatgcttggtatgcagttgaggcagctttatgtttccatacccttccacttcggctcggggactcggcggggtcgcggaagtttgacggcagccgtcgggccttaggccgagattaccgtggttgtggcccagttttagccgttcccgtccgtttcctaccggaccatgattttcgtgaaccattgcaatcccgaagcgcatttccgacgttaaggagttacctccgctgcccacaattcatgatcgtggccggctcaaggcagcgtggcggggcatccgtgtcaagctcccaggaggaggtgcgcgatttcaaatccgggccaaaacaggccaagactggctggccaaaaaaaggagcgtagacggcccgggacatcggacgtcagctcgcagccacccaaaaccggtccgatctactcgcttactgtggtagttcaggtacttttgagtagtaaaaacgctacggcagggccggggggttccccggtgacggaggtgcctctgcggtggcgaacatcccacgcactatcgagctacggtgacacctcgtgtcctgttggtcttgcaatgctggggcggcaggaaatgcgtcgcgctcctcccggccaagacctaaaacagacagcgccgcaaagtcgctcactagcaccgcgaaacgaagatgccccacctcaacgcaatctgtgatgcaagcaattgggaaggctcaccccacctcagcgaggggctcaaccatttttattatcagctcatgccaccacaacatgactgttttctttccttgctcatcccacatttgacaaaaatcgtcgattaatctctttccatacaggccgtccgcgctctgataaccacataaaagtctcttcagtcaacagctcaaagctccctcatccctccaggtaagcagccaaagagctcccccacggaccccgcactgcctcatcccgcctgtatcggacctgcgcgacccagcagagaatcccaaacctttgctgcttgctgcccggttccggactgagctgcaacccaagcctttaaaaagctattcccttctcccacggtgtcaactctgtcctatccctccgacatccgttgagctcaacaactccccgaaccttttaccccgcgccgagctacccctccatcaaaccaccctgacagctcgctcactcacctccccacatcacagaaatcaaa

SEQ ID NO: 16 - DNA, 5188 bp, Myceliophthora thermophila
  Donor DNA fragment donor-cdt1
caggatattgataacacctttaattttcaagacgcctaattaaaaagccatcatccatgagcggaaacgagaagccgacgtcgcggatcgcgatcgtgggggtgggccaggtgggcgcggcggcggcgtacgcgctgatcctcgagtcggtcgcgggcgagctgctgctggtggacatcaaagcagactggcgagacgggcaggtacgcgatctggccgacgtggcgtacggcatcggcagcggcacgcgcgtgcgggcggcgacgcaccgcgaggcgggccagtgcgacattgtggtcatcaccgccggctccaagtggtccatcggtcagtttctgattaattattaattttctcatctcaggggatgcaaaaaggggctcacgcatgagaataattaattaggcgagacgaacgtccagcacatgcaccgcaacgtgtcggtcatgcgcagcgtgatgcgggacatgacgccgttccggtccgacgctatcgtgatcgtggtgtcaaacccggtcgacctgctcacctccatcgcgcaggagctctcggggctcccgaggttccaagtgtttggctcgggacgaggacttctagagatcgttgcatcgtcccaaagctattggcgggatattctgtttgcagttggctgacttgaagtaatctctgcagatctttaattaactcgagtgacggtgcttttcacctctcgatgcccgaaatcgggtctaagctgagtttgatcaaatatgtgactccaacatcgcccccttcggcaaaccccgtcgacacgtgtgtcatccttccattgcaagcgatcactcgcagggcgtgacgatgaacgagatttttgcccggaccgattcgcggatatagcggcagccgaccagccctaccacactgatggccgtgtccctagtgtatgctcccagaaccgcaagcatacactgggcaatgcttggtatgcagttgaggcagctttatgtttccatacccttccacttcggctcggggactcggcggggtcgcggaagtttgacggcagccgtcgggccttaggccgagattaccgtggttgtggcccagttttagccgttcccgtccgtttcctaccggaccatgattttcgtgaaccattgcaatcccgaagcgcatttccgacgttaaggagttacctccgctgcccacaattcatgatcgtggccggctcaaggcagcgtggcggggcatccgtgtcaagctcccaggaggaggtgcgcgatttcaaatccgggccaaaacaggccaagactggctggccaaaaaaaggagcgtagacggcccgggacatcggacgtcagctcgcagccacccaaaaccggtccgatctactcgcttactgtggtagttcaggtacttttgagtagtaaaaacgctacggcagggccggggggttccccggtgacggaggtgcctctgcggtggcgaacatcccacgcactatcgagctacggtgacacctcgtgtcctgttggtcttgcaatgctggggcggcaggaaatgcgtcgcgctcctcccggccaagacctaaaacagacagcgccgcaaagtcgctcactagcaccgcgaaacgaagatgccccacctcaacgcaatctgtgatgcaagcaattgggaaggctcaccccacctcagcgaggggctcaaccatttttattatcagctcatgccaccacaacatgactgttttctttccttgctcatcccacatttgacaaaaatcgtcgattaatctctttccatacaggccgtccgcgctctgataaccacataaaagtctcttcagtcaacagctcaaagctccctcatccctccaggtaagcagccaaagagctcccccacggaccccgcactgcctcatcccgcctgtatcggacctgcgcgacccagcagagaatcccaaacctttgctgcttgctgcccggttccggactgagctgcaacccaagcctttaaaaagctattcccttctcccacggtgtcaactctgtcctatccctccgacatccgttgagctcaacaactccccgaaccttttaccccgcgccgagctacccctccatcaaaccaccctgacagctcgctcactcacctccccacatcacagaaatcaaaactagaatgtcgtctcacggctcccatgacggggccagcaccgagaagcatcttgctactcatgacattgcgcccacccacgacgccatcaagatagtgcccaagggccatggccagacagccacaaagcccggtgcccaagagaaggaggtccgcaacgccgccctatttgcggccatcaaggagtccaatatcaagccctggagcaaggagtccatccacctctatttcgccatcttcgtcgccttttgttgtgcatgcgccaacggttacgatggttcactcatgaccggaatcatcgctatggacaagttccagaaccaattccacactggtgacactggtcctaaagtctctgtcatcttttctctctataccgttggtgccatggttggagctcccttcgctgctatcctctctgatcgttttggccgtaagaagggcatgttcatcggtggtatctttatcattgtcggctccattattgttgctagctcctccaagctcgctcagtttgtcgttggccgcttcgttcttggcctcggtatcgccatcatgaccgttgctgccccggcctactccatcgaaatcgcccctcctcactggcgcggccgctgcactggcttctacaactgcggttggttcggaggttcgattcctgccgcctgcatcacctatggctgctacttcattaagagcaactggtcatggcgtatccccttgatccttcaggctttcacgtgccttatcgtcatgtcctccgtcttcttcctcccagaatcccctcgcttcctatttgccaacggccgcgacgctgaggctgttgcctttcttgtcaagtatcacggcaacggcgatcccaattccaagctggtgttgctcgagactgaggagatgagggacggtatcaggaccgacggtgtcgacaaggtctggtgggattaccgcccgctcttcatgacccacagcggccgctggcgcatggcccaggtgctcatgatctccatctttggccagttctccggcaacggtctcggttacttcaataccgtcatcttcaagaacattggtgtcaccagcacctcccaacagctcgcctacaacatcctcaactccgtcatctccgctatcggtgccttgaccgccgtctccatgactgatcgtatgccccgccgcgcggtgctcattatcggtaccttcatgtgcgccgctgctcttgccaccaactcgggtctttcggctactctcgacaagcagactcaaagaggcacgcaaatcaacctgaaccagggtatgaacgagcaggatgccaaggacaacgcctacctccacgtcgacagcaactacgccaagggtgccctggccgcttacttcctcttcaacgtcatcttctccttcacctacactcccctccagggtgttattcccaccgaggctctcgagaccaccatccgtggcaagggtcttgccctttccggcttcattgtcaacgccatgggcttcatcaaccagttcgctggccccatcgctctccacaacattggctacaagtacatctttgtctttgtcggctgggatcttatcgagaccgtcgcttggtacttctttggtgtcgaatcccaaggccgtaccctcgagcagctcgaatgggtctacgaccagcccaaccccgtcaaggcctccctaaaagtcgaaaaggtcgtcgtccaggccgacggccatgtgtccgaagctatcgttgcttaggatatcgaattcggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggccgctggacaggttcgtggctccagtcgtgtcgggcgtggaactcatgcacgcggacctcgaggacgagtgcaagttccgttccgagcacatcatccgggccaaggggggcacaccttttggcatcggctcggtcgtggccagcctctgctcgaccgtcctccgggacaagggcgacgtgcggccggtcagctactaccagcccgactatggctgctgcttcagcctgcccgtggttctggggaggaagggcatcatgcggacgatcgcgatgccgctggacgagcgtgagaaggccacggtgtcccggtcggcccagaggctcaaggagacgattgagcgattgcacaagagccagtgagagtgcagtacggatacataggtacagtatgtacatacatgttatgtacggaccgtgctagtaaggttggtacttacttgtaggtctcgttagcttgtcaaaaaaaaaaaaaaaaaaaaaaaaaaaaaaaaaaaaagggggggggggcgggggggggggggaggttgaagtcaggggtgtcaggggttaacaacagatgggc

SEQ ID NO: 17 - DNA, 730 bp, Myceliophthora thermophila
  promoter of cdt-2
tcaagtccagcatagcgacaaccaaggagcattcgatatgcaacccgatctcaagcagagaaaggtgttagggccgacgaaaagaccagtcttgcgagccaacctgcgctggacctatggtactgcgtgtgtgtgggcggataccgagtgtactccgtaaggagggttggtctcatgcctcttggcgggagccgcccgataactagtataactagttgtaactccgtatccggttacggaaacggaaaggcccgctcggctgttctccggcggctccccgatcgctgatcagagcatggaacagatgtcaattacatcactcccgcgtaaacgaaccatagttatcgaaccacagttatcgaaccacagagccagcccatgggaacgtctgaacagctcggaggatgcaaccgatattgcaatgcaaaacgtcacccatgctacaattaattccctgcacaactacttgtaagccgcgaggcctagaacacagttgcagaacctgggtatcgtgcctgtggtctgatgcagatatgtgtcaccactcaagaccccgccaacacgccgcttcgaggccctgaacagtacaaagggcgcttcaaattcgtacaagcccccccgaggccgttttcaagtctttgtatgaccatctattttccgattgacgtccctcacggattctctttcgttgctgacctccttgtgaccacaaacatcgccaacaacagac

SEQ ID NO: 18 - DNA, 28 bp, Artificial sequence
  Primer
tcatcttctgtggttgtggtggtggtgg

SEQ ID NO: 19 - DNA, 30 bp, Artificial sequence
  Primer
taccgtcaaaatgtctgaaattactttggg

SEQ ID NO: 20 - DNA, 5496 bp, Myceliophthora thermophila
  The donor DNA fragment donor-cdt2 of cdt-1 gene
cgtcaggtgtctgttctcgcgaaatacatataatctcctcggttagatttgttcaggagaaagatgatggtgccaaactctacagtttaggcttctctaccgaggaaagagaatgagaggtcggaccaccagcgtgctaacaggacgagacttgagccaatttgtcaatagacaagatagtctcgtgtgccaagccaacatcatcatccgatctcagctgagcccctccttctgaaaagagtataattacagcagcttcagacgttgaggcatcactgttgctgccgcacgtcagtcaaccacttaatccgctctgtgtgaccggtgacggattcgatcattctacgatccgctccgcatcgccggttaaagggggtgacgagttatttattacctttacggattcaagatgcctcagccaaacgcagcagatgtcaaggtcagtgaagaggattgtttcgaattactcagaaatgaaacacctgcttacctaggtacacgttctcatgctccgcccaacggatagtcaatcaagatcgttatcgtcggtgccggctcggtaggcgtgacgactgcgtactcaagtccagcatagcgacaaccaaggagcattcgatatgcaacccgatctcaagcagagaaaggtgttagggccgacgaaaagaccagtcttgcgagccaacctgcgctggacctatggtactgcgtgtgtgtgggcggataccgagtgtactccgtaaggagggttggtctcatgcctcttggcgggagccgcccgataactagtataactagttgtaactccgtatccggttacggaaacggaaaggcccgctcggctgttctccggcggctccccgatcgctgatcagagcatggaacagatgtcaattacatcactcccgcgtaaacgaaccatagttatcgaaccacagttatcgaaccacagagccagcccatgggaacgtctgaacagctcggaggatgcaaccgatattgcaatgcaaaacgtcacccatgctacaattaattccctgcacaactacttgtaagccgcgaggcctagaacacagttgcagaacctgggtatcgtgcctgtggtctgatgcagatatgtgtcaccactcaagaccccgccaacacgccgcttcgaggccctgaacagtacaaagggcgcttcaaattcgtacaagcccccccgaggccgttttcaagtctttgtatgaccatctattttccgattgacgtccctcacggattctctttcgttgctgacctccttgtgaccacaaacatcgccaacaacagacatgggcatcttcaacaagaagcccgtggctcaggccgtcgacctcaatcagatacaggaggaggctcctcagtttgagagggttgactggaagaaggaccccggtcttcgcaagctctacttctacgccttcattctttgcattgcttcggccaccaccggttacgatggcatgttcttcaactcggtgcagaacttcgagacctggattaagtactttggcgacccgcgaggatccgagcttggtctgctcggtgctctctaccagattggttccattggctccatcccctttgtccccctccttaccgacaactttggccgcaagacccccatcatcatcggctgcgttatcatgatcgtcggtgccgttctccaggccacggccaagaacctcgatacattcatgggcggccgtaccatgctcggctttggcaactccctcgcccagatcgcctcccccatgcttctcaccgagctcgcccatcctcaacaccgcgctcgtctcaccaccatctacaactgcttgtggaacgttggtgccctcgtcgtctcgtggttggcctttggcaccaactacatcaacaacgactggtcatggcgcattcccgccttgctccaggctttcccctccatcattcagctcctcggtatctggtgggttcccgagtctccccgtttcctcatcgccaaggacaagcacgacgaggccctccacatcctcgccaagtaccacgccaacggcgaccccaaccaccccaccgtccagtttgagttccgcgagatcaaggagaccatccgcctcgagatggaatcgaccaagaacagcagctacctcgacttcttcaagagccgcggcaaccgctaccgcctcgccatcctcctctcgctcggcttcttctcccaatggtccggcaacgccatcatctccaactactcctccaagctgtacgagaccgccggcgtcaccgactccaccgccaaactcggtctttccgccggacagaccggtctcgcgctcatcgtgtccgtcaccatggcgctgctcgtcgacaagctcggtcgtcgtcttgctttcctcgcttccacgggcggcatgtgcggcacctttgtcatttggacgttgacagccggcctgtacggcgagcaccgcctcaagggcgccgacaaggccatgatcttctttatctgggtgttcggcatcttctactcgctcgcctggtccgggttgctggtcggctacgccatcgaaatcctcccttaccgacttcgcggcaaggggttgatggtcatgaacatgtcggtgcagtgcgcgctgacgctcaatacttatgcgaaccctgttgcgtttgattactttggtcctgatcactcgtggaagctttatcttatttacacttgctggatcgccgccgagttcgtcttcgtcttcttcatgtacgtcgagaccaagggccccacgctcgaggagcttgccaaggtcattgatggcgatgaggccgatgttgcccacatcgacattcaccaggtcgagaaggaggtggagattcacgagcatgagggcaagtctgttgcttgagatatcgaattcgggatccacgaacctctctgaaggaggttctgagacacgcgcgattcttctgtatatagttttatttttcactctggagtgcttcgctccaccagtacataaaccttttttttcacgtaacaaaatggcttcttttcagaccatgtgaaccatcttgatgccttgacctcttcagttctcactttaacgtagttcgcgtttgtctgtatgtcccagttgcatgtagttgagataaatacccctggaagtgggtctgggcctttgtgggacggagccctctttctgtggtctggagagcccgctctctaccgcctaccttcttaccacagtacactactcacacattgctgaactgacccatcatactcgacagaagatgatattgaagaagcactttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtgaacgacccggtcataccttcttaagttcgcccttcctccctttatttcagattcaatctgacttacctattctacccaagcaaagcttcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggactgcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgaacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtattccgggacgtagtgccccgcatcgcacgccacgcgcccgacaccatcctcgtcgtggccaccaacccggtcgacgtgctcacgcacgcggcccaccacctctccggcttcccactcgagagggtgatcgggtccggcacggccatggacaccaccaggttccggcacgagctgggcaagcactttggggtcaacccgcggaacgtgcacgccatgatcgtgggcgagcacggcgacagccagctgcccgtgtggtcgctcgccaccatctgcgggatgcggctgcacgactactgcagggcggcccgcatggagcacgacgaggccgcactcgaggcctgcgccaagcggaccagggaggccgcctacgagatcatccggcgcaagggcaagaccaactacggcgtcgcctcggtgctcgtcagcatcctgcagcccatcgtcaccgacagcgacgccatcatgacggtctcgagggtcggcacgtacgccggaatccaggacgtggccctcagcatgccctgcaagctgaaccggcatggcgcgtaccaggacgtgcccctactcctcagcgagttggag

SEQ ID NO: 21 - DNA, 31 bp, Artificial sequence
  Primer
accacaaccacagaagatgatattgaaggag

SEQ ID NO: 22 - DNA, 32 bp, Artificial sequence
  Primer
gtcttcttctgaaagaaggattacctctaaac

SEQ ID NO: 23 - DNA, 30 bp, Artificial sequence
  Primer
tccttctttcagaagaagacgttcgaggtc

SEQ ID NO: 24 - DNA, 23 bp, Artificial sequence
  Primer
tccatgtgcgacgagagggcggc

SEQ ID NO: 25 - DNA, 45 bp, Artificial sequence
  Primer
gatttcagtaacgttaagtggatccttattgcttagcgttggtag

SEQ ID NO: 26 - DNA, 567 bp, Myceliophthora thermophila
  sgRNA expression plasmids U6p-mpd-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcgacttggttacctgctccgtgttttagagctagaaatagcaagttaaaataaggctagtccgttatcaacttgaaaaagtggcaccgagtcggtgcttttttt

SEQ ID NO: 27 - DNA, 2197 bp, Myceliophthora thermophila
  the donor DNA fragments donor-ldh2
agtttatgtggaaaatggcatttgaaatgaggggtattcggaatctagggcgtttgctactgcgattagatgacgtcaggtgtctgttctcgcgaaatacatataatctcctcggttagatttgttcaggagaaagatgatggtgccaaactctacagtttaggcttctctaccgaggaaagagaatgagaggtcggaccaccagcgtgctaacaggacgagacttgagccaatttgtcaatagacaagatagtctcgtgtgccaagccaacatcatcatccgatctcagctgagcccctccttctgaaaagagtataattacagcagcttcagacgttgaggcatcactgttgctgccgcacgtcagtcaaccacttaatccgctctgtgtgaccggtgacggattcgatcattctacgatccgctccgcatcgccggttaaagggggtgacgagttatttattacctttacggattcaagatgcctcagccaaacgcagcagatgtcaaggtcagtgaagaggattgtttcgaattactcagaaatgaaacacctgcttacctaggtacacgttctcatgctccgcccaacggatagtcatattgaaggagcactttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtgaacgacccggtcataccttcttaagttcgcccttcctccctttatttcagattcaatctgacttacctattctacccaagcaaagcttcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaagattgaatcctgtactagtgatccgtcggcgactacgaggactgcgcccacgcggcggcggtcatcatcacggcgggcgtcaaccaaaagcccggccagacgcgcctcgacctcgtcaagaccaactacgcgctattccgggacgtagtgccccgcatcgcacgccacgcgcccgacaccatcctcgtcgtggccaccaacccggtcgacgtgctcacgcacgcggcccaccacctctccggcttcccactcgagagggtgatcgggtccggcacggccatggacaccaccaggttccggcacgagctgggcaagcactttggggtcaacccgcggaacgtgcacgccatgatcgtgggcgagcacggcgacagccagctgcccgtgtggtcgctcgccaccatctgcgggatgcggctgcacgactactgcagggcggcccgcatggagcacgacgaggccgcactcgaggcctgcgccaagcggaccagggaggccgcctacgagatcatccggcgcaagggcaagaccaactacggcgtcgcctcggtgctcgtcagcatcctgcagcccatcgtcaccgacagcgacgccatcatgacggtctcgagggtcggcacgta

SEQ ID NO: 28 - DNA, 1200 bp, Myceliophthora thermophila
  the donor DNA fragments donor-mpd
cctgactgtataagagaaatggcctcccctcggagtctgagcgcttcctttcctttatctgcagacttttcagtcatttcatcttctttatcctaaggttttctgccaactcgtattaaggcccttgccgttgttaagaacacacgtcatcatctcgtcttcgactgcctttcctattgcaccgagattctgagccgtcgatcatctatctctctttcgacttccttcttcaaccctcaacctactcaacacatcctaccttccaaaatggcgcaaaggaatgcccccaagaaggccgtccacttcggtgctggaaacatcggtatgaatcctcttgggagattggcatcgacgaccgaccttgactgaccgtgatgatttccaggtcgtggcttcgtcgcctgctttttgcacaattcaggctacgaggtcgtcttctctgaggttaacgacgcaactgtatccaagctcaactcccaaaagagctatcgggtgattgaagtcggcgccgacgggacgtcggagtccattatcaccaattaccgcgccatcaactcgaggacccacgaggccgaactggttcgggaaattgccacggcgggagcttactccggtcgctgtcattgcctgcgagaacgccatcggggccaccgatactcttgctggtttcatcaagtcgccagagaacaccaatcctgaccggcttgccgactacgataagcgtgctcggttcgccaactccgccatcgaccgcatcgtccccgcacaggaccccaacgccggtctcgacgttaggctcgagaagttctacgagtgggtggtagacagcaccccatttgccgaccatgccccccccgacatcaagggtgttcgctgggtgaacaacctgcagcccttcatcgagcgcaagctgtacacggtcaacacgggccacgccacggccgcgtaccacggctacatccggcgtaagagcaccgtctacgacgcgctgcaagaccgcgagattcaagaagaggtcaagaaggcgctgcagaacacggccgacctcatcacgcagaagcacggcatctcgcacgaggagcagcaggcctacgtcgagaagattgtccgccgtatcggcaatcctcacctcgaggacgccgtcgagcgcgtcggccgcgccccgttgcgcaagctcagccgtaaggaacgcttcattgg

SEQ ID NO: 29 - DNA, 6134 bp, Artificial sequence
  cas9 expression frame
  misc_feature (5718)..(5718): n is a, c, g, or t
  misc_feature (5926)..(5926): n is a, c, g, or t
  misc_feature (6017)..(6017): n is a, c, g, or t
tcctccgaggttcgacatcagggttcgtcatagggagtgaaacacccgccatgattccgtagccgcgcgcgaagatacgaagcagatatttcacggacatggcggagatacttgtttcccgtactaaggtagtcatgtcggagacatctgaacgacagagctggccaagagaaccgaccagttgccccaggacgatctagacaaaaaaaaagagagatgagtgggccacttttgccacaacatcgacggccctgcgaccgcccccaggcaaacaaacaaaccgccgaacaataatacttttgtcattttaggaggagcgttgtatggataaaaacaacatctcgttgctgcagaatgtggacttcaaacttgcagaaaatgggaggcggatttgcatgatcggagggtagttgactcacgccgcaggctgcaaatccgtcctccattattccatgaacaacttcgtaaggttgggctgagcgccaatgcctaacggaccgggggccacagcgcaacgtcccacttaaaggccagcgtgacatgccagttccataccaagtagtggcaccagaggcggccaatgctcagtaagggcagggagggaggctcaaacgattggcaaaaagaggggcttgccagttcagttccctgtgcgagcgcgagaggggcagtttcaaatctggaggggtgtgttgcgctggtctgaagagaaagagaagactgtacttaataattgttcaaagagtccatcatcgcgttgcggactcctctagctgtatttagagccctatcattacttgtcgggtgcgaatcaaaataccgggatgcagccctctggcgatttgcatgcggttgtggaggaagtgaagcctgaatcgcggggctgggcggcaaagcacgacgtgaaattcctggcgaaattcgagggcttgccccaccgtggttgaagtttttgtgctgcgtaaccccaccaacccgccttgcccctcccgcctgcccataaaaacttcgacccctcctcaaatcttcttcgattcttcctcttcacttccttcgtcggcatacctgattcaagcaatcacctgccactttcaagtgcgtataccatcatcgatacactggttcttgacaagtacatcgtctctaactttcctttttgcagttttcattaagcgcaagtcgccagtttcgttcttcagaaagcttatggactacaaggaccatgatggcgattacaaggaccacgacatcgattataaggatgatgatgacaagcctccgaggaaacgtgccaaaacagaagatgagatggataagaagtactccatcggcctcgacatcggcaccaactccgtcggctgggccgtcatcaccgatgagtacaaggtcccttccaagaagttcaaggtcctcggcaacaccgatcgccattccatcaagaagaacctgatcggcgccctcctgttcgattccggcgaaaccgccgaggccacccgccttaaacgcaccgcccgtcgccgctacacccgccgcaagaaccgcatctgctacctccaagaaatcttctccaacgagatggccaaggtcgatgatagcttcttccaccgcctcgaagagtccttcctggtcgaagaggataagaagcacgagcgccatcctatcttcggcaacatcgtcgatgaggtcgcctaccatgagaagtaccctaccatctaccatctccgcaagaagctcgtcgattccaccgataaggccgatctccgcctcatctacctcgccctcgcccatatgatcaagttccgcggccatttcctcatcgagggcgatctcaaccctgataactccgatgtcgataagctgttcatccagctcgtccagacctacaaccagctgttcgaggaaaaccctatcaacgcctccggcgtcgatgccaaggccatcctctccgctcgcctctccaagtctcgccgccttgagaaccttatcgcccagctccctggcgagaagaagaacggcctcttcggcaacctgatcgccctctccctcggcctcacccctaacttcaagtccaacttcgatctcgccgaggatgccaagctccagctctccaaggatacctacgatgatgatctcgataacctcctcgcccagatcggcgatcagtacgccgatctgttcctcgccgccaagaacctctccgatgccatcctcctctccgacatcctccgcgtcaacaccgagatcaccaaggcccctctgtccgcctccatgatcaagcgctacgatgagcatcatcaggacctcaccctgctcaaggccctcgtccgccagcagctccctgagaagtacaaagagattttcttcgatcagtccaagaacggctacgccggctacatcgatggcggcgcttcccaagaagagttctacaagttcatcaagcctatccttgagaagatggatggcaccgaggaactcctcgtcaagctcaaccgcgaggacctcctccgcaagcagcgcaccttcgataacggctccatccctcatcaaatccatctcggcgagctgcatgccatcttgcgccgccaagaggatttctacccattcctcaaggataaccgcgagaagatcgaaaagattctcaccttccgcatcccttactacgtcggccctctcgctcgcggcaactcccgcttcgcctggatgacccgcaagtccgaggaaaccatcaccccttggaacttcgaggaagtcgtcgataagggcgcctccgcccagtccttcatcgagcgcatgaccaacttcgataagaacctccctaacgagaaggtcctccctaagcactccctgctctacgagtacttcaccgtctacaacgagctgaccaaggtcaagtacgtcaccgagggtatgcgcaagcctgccttcctgtccggcgagcagaagaaggccatcgtcgatctgctgttcaagaccaaccgcaaggtcaccgtcaagcagctcaaagaggattacttcaagaaaatcgagtgcttcgattccgtcgagatcagcggcgtcgaggaccgcttcaacgcctccctcggaacctaccatgatctcctcaagattatcaaggataaggatttcctcgacaacgaggaaaacgaggacatccttgaggacatcgtcctcaccctcaccctcttcgaggaccgcgaaatgatcgaggaacgcctcaagacctacgcccatctcttcgatgataaggtcatgaagcagctcaagcgccgtcgctacaccggctggggtcgcctctcccgcaagctcatcaacggcatccgcgataagcagtccggcaagactatcctcgatttcctcaagtccgatggcttcgccaaccgcaacttcatgcagctcatccatgatgattccctcaccttcaaagaggacatccagaaggcccaggtcagcggccagggcgattccctccatgagcatatcgccaacctcgccggctcccctgccatcaagaagggcatcctccagaccgtcaaggtcgtcgatgagctggtcaaggtcatgggccgccataagcctgagaacatcgtcatcgagatggcccgcgagaaccagaccacccagaagggccagaagaactcccgcgagcgcatgaagcgcatcgaggaaggcatcaaagagctgggcagccaaatcctcaaagagcatcctgtcgagaacacccagctccagaacgagaagctctacctctactacctccagaacggccgcgatatgtacgtcgatcaagagctggacatcaaccgcctctccgattacgatgtcgatcatatcgtccctcagtccttcctgaaggatgattccatcgataacaaggtcctcacccgctccgataagaaccgcggcaagtccgataacgtcccttccgaagaggtcgtcaagaagatgaagaactactggcgccagctcctcaacgccaagctcatcacccagcgcaagttcgataacctcaccaaggccgagcgcggtggcctctccgagctggataaggccggcttcatcaagcgccagctcgtcgaaacccgccagatcaccaagcacgtcgcccaaatcctcgattcccgcatgaacaccaagtacgatgagaacgataagctcatccgcgaagtcaaggtcatcaccctcaagtccaagctcgtcagcgatttccgcaaggatttccagttctacaaggtccgcgagatcaacaactaccatcatgcccatgatgcctacctcaacgccgtcgtcggcaccgccctcatcaagaagtaccccaagctcgaatccgagttcgtctacggtgattacaaggtctacgatgtccgcaagatgatcgccaagtccgagcaagagatcggcaaggctaccgccaagtacttcttctactccaacatcatgaatttcttcaagaccgaaatcaccctcgccaacggcgaaatccgcaagcgccctctcatcgagactaacggcgagactggcgagatcgtctgggataagggccgcgatttcgccaccgtccgcaaggtcctctccatgcctcaggtcaacatcgtcaagaaaaccgaggtccagaccggcggcttctccaaagagtccatcctccccaagcgcaactccgataagctgatcgcccgcaagaaggattgggaccctaagaagtacggcggcttcgattcccctaccgtcgcctactccgtcctcgtcgtcgccaaggtcgagaagggcaagtccaagaagctcaagtccgtcaaagagctgctcggcatcactattatggaacgctccagcttcgagaagaaccctatcgatttccttgaggccaagggctacaaagaggtcaagaaggacctcatcatcaagctccccaagtactccctgttcgagcttgagaacggccgcaagcgcatgctcgcctccgccggtgagcttcagaagggcaacgagctggccctgccttccaagtacgtcaacttcctctacctcgcctcccattacgagaagctcaagggctcccctgaggataacgagcagaagcagctgttcgtcgagcagcataagcactacctcgatgagatcatcgagcagatcagcgagttctccaagcgcgtcatcctcgccgatgccaacctcgataaggtcctgtccgcctacaacaagcaccgcgataagcctatccgcgagcaggccgagaacatcatccatctcttcaccctcaccaacctcggtgcccctgccgccttcaagtacttcgataccaccatcgatcgcaagcgctacacctccaccaaagaggtcctggacgccaccctcatccatcagtccatcaccggcctctacgaaacccgcatcgatctctcccagctcggcggcgaccctccgaggaaacgtgccaaaacagaagatgagtgatgaggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccaga

SEQ ID NO: 30 - DNA, 45 bp, Artificial sequence
  Primer
tctgcagatctttaattaactcgagcacccgccatgattccgtag

SEQ ID NO: 31 - DNA, 37 bp, Artificial sequence
  Primer
tttcagacattttgacggtatttgtgttctgaagaac

SEQ ID NO: 32 - DNA, 567 bp, Myceliophthora thermophila
  sgRNA expression plasmid U6p-mdh-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcggctacctgcccgccaacgagttttagagctagaaatagcaagttaaaataaggctagtccgttatcaacttgaaaaagtggcaccgagtcggtgcttttttt

SEQ ID NO: 33 - DNA, 2373 bp, Myceliophthora thermophila
  the donor DNA fragment donor-mdh
gggtggtgtgcatggggccgtctcggcggggtgtgtgaacgcggggcagaactgagtgttcccgcggttcgacgggccttcaaagcaagctgccccacgaccccgccaactctctgagcatgacttaggtacttaagctaccaaagcttcgagtctgcaacatcgtgaccttccaactttcccagcgacttacttgtttcaaccattgcatcactttccaggtcagtgctccagtctaatattcgcactctgcgaactccaattgcatttttacaatcgagagctgaccctcagactgcgagacgtcaacagttcccccacgatcctccgctgcctccgatcctaactccaactccaaacaaaatggtcaaagctggtaagcacacgacgccatcttctgggcgatgtaggcatcaactggtctgacattgttcctaaacagtcgttgctggcgcctccggcggtattggacaggttcgtatgccctcaagctctctggagccagcccgctgacgccatcggtaccccgcagccgctgtcgctgctcctgaagctctctccgctcgtcagcgagctcgcactctacgatgttgtcaacacccccggtgttgcgaccgacctgtcgcacatttcatccaacgccgtaagtggaaaagccgttaaacccggcagcgttctcagacccggctgaccctgctgcatattgaaggagcactttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtgaacgacccggtcataccttcttaagttcgcccttcctccctttatttcagattcaatctgacttacctattctacccaagcaaagcttcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaaaagcccggcatgacccgtgatgacctcttcaacatcaacgccggcatcgtgaagggcctcattgaggtggccgccgaggtcgcccccaaggccttcatcctggttatttccaaccccgtcaactcgaccgtccccatctcggccgaggtgctcaaggccaagggcgtcttcaacccccagcgcctcttcggtgtcaccaccctcgacattgtccgtgccgagaccttcgttgccgagattgccggcaagagcaacccccaggagttgactgtccccgtcgttggcggtcactccggcgagaccattgttccgcttttcagcaagatcgctccgtctgtcacgattccggacgacaagtacgacgctcttgtcaaccgcgtccagttcggtggtgatgaggttgtcaaggccaaggacggcgctggttctgccacactttcgatggcgtatgctggttacaggtaagcgccagacagtaaccacccttcgacttctatttttgctaaccgctatcaaggttcgccgagaagctcctcaaggccgccgcgggcgtcaagggcctcgttgaaccaagctacgtctaccttcccggcatcccaggtggcaaggagattgccgagaagaccggtgtcgagttcttctccgttcccatcgagcttggcgtaagtcacaaagaggatagaggaagagcttcagtacg

SEQ ID NO: 34 - DNA, 567 bp, Myceliophthora thermophila
  sgRNA expression frame U6p-gpd-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcggagaatgtcacaatccccggttttagagctagaaatagcaagttaaaataaggctagtccgttatcaacttgaaaaagtggcaccgagtcggtgcttttttt

SEQ ID NO: 35 - DNA, 1400 bp, Myceliophthora thermophila
  donor DNA expression vector donor-gpd
tgggacggcgagagcgacgggttttggccgaggtgggcgggttcgtcaacggccttggcgctgaacgactgaaggcggtcaatgttttctgccgccccttgcgtcatggctcgcagaatgggccaacggaatttcgtaccaggaggaaaatttccttgtttggcggaaaaccgccgggagggggtcgtctgatgaaccacgaggcgatgcacccagaaggctgcacaggtagagcgtttttgcctacatagctgtgttcagttttcttgagtttgctgtgtaagggtccaggatggtacggggcgggcttttgcggccgcccctactgcccacataccagagcagcggtgcaattttgtcggcgttttcctgcgacccatcgtccctccccagcgtcagacaccggtcaccgcctgcctttttttctacgcccaacgtgacccatttccaccacaggcctacccacttaagaggctgcacgttccgactgcccaagcccaaaccttgtcagccaagtcggttcttcatatgccaagcgtctcaccgtcccaatctttcttcctgctctcgtccgaagtctttgccggcaatccgtggccgctctctcaaacagacaaactcggctcccgctctcattccccctcgcagcatccgtctgttatcgcacacagcaaaaatggcacgatccacagactctgccttctcgacgccgtcagggactcttccattcttatcttcaacctcccgcacgagttcatcggcaacatttgcaagcaaatcaagggtcatatcctcccctttgcccggggaatcagctgcatcaagggggtaacagtgacggaggacaaggtcgagctgatttgcgagtatattggcgagacgctgggtatttactgcggcgccctgagcggggccaacattgccaatgagatcgccaacgagctatggtgcgagacgaccatcgcatacaacgtaccaccgtgcgactcgcggggcgagaacggcgagagcggcgtcaatggagagcactatcgtgacgcgcgcggtgcaatcagcaaaacgcagctcgctcccgtacctcaggagtacccgccagtggaccatgacgtgctccacaagctctttaaccggccttattttaccgtctccatggtgtcggacgtggttggtgtctcgctcgccggtgcgctcaagaacatcgttgcgctcgcctgcggcttcattgaaggtcacggttggaacatgacggctaagacggccgttatgcgccgtggcatgctggagacgattcagttctgccaagaattcttccccgagacggtcgacgagatgacgttctgggagagcgccggctggagcgacatgattgtgagctgcacctcggcacgcaactggcgcta

SEQ ID NO: 36 - DNA, 40 bp, Artificial sequence
  Primer
aagaggagtccaccatggtactcgacagaagatgatattg

SEQ ID NO: 37 - DNA, 28 bp, Artificial sequence
  Primer
aacgttaagttcagatctcggtgacggg

SEQ ID NO: 38 - DNA, 4227 bp, Artificial sequence
  dCas9 nucleic acid fragment
aaggttgataagaagtactccatcggcctcgccatcggcaccaactccgtcggctgggccgtcatcaccgatgagtacaaggtcccttccaagaagttcaaggtcctcggcaacaccgatcgccattccatcaagaagaacctgatcggcgccctcctgttcgattccggcgaaaccgccgaggccacccgccttaaacgcaccgcccgtcgccgctacacccgccgcaagaaccgcatctgctacctccaagaaatcttctccaacgagatggccaaggtcgatgatagcttcttccaccgcctcgaagagtccttcctggtcgaagaggataagaagcacgagcgccatcctatcttcggcaacatcgtcgatgaggtcgcctaccatgagaagtaccctaccatctaccatctccgcaagaagctcgtcgattccaccgataaggccgatctccgcctcatctacctcgccctcgcccatatgatcaagttccgcggccatttcctcatcgagggcgatctcaaccctgataactccgatgtcgataagctgttcatccagctcgtccagacctacaaccagctgttcgaggaaaaccctatcaacgcctccggcgtcgatgccaaggccatcctctccgctcgcctctccaagtctcgccgccttgagaaccttatcgcccagctccctggcgagaagaagaacggcctcttcggcaacctgatcgccctctccctcggcctcacccctaacttcaagtccaacttcgatctcgccgaggatgccaagctccagctctccaaggatacctacgatgatgatctcgataacctcctcgcccagatcggcgatcagtacgccgatctgttcctcgccgccaagaacctctccgatgccatcctcctctccgacatcctccgcgtcaacaccgagatcaccaaggcccctctgtccgcctccatgatcaagcgctacgatgagcatcatcaggacctcaccctgctcaaggccctcgtccgccagcagctccctgagaagtacaaagagattttcttcgatcagtccaagaacggctacgccggctacatcgatggcggcgcttcccaagaagagttctacaagttcatcaagcctatccttgagaagatggatggcaccgaggaactcctcgtcaagctcaaccgcgaggacctcctccgcaagcagcgcaccttcgataacggctccatccctcatcaaatccatctcggcgagctgcatgccatcttgcgccgccaagaggatttctacccattcctcaaggataaccgcgagaagatcgaaaagattctcaccttccgcatcccttactacgtcggccctctcgctcgcggcaactcccgcttcgcctggatgacccgcaagtccgaggaaaccatcaccccttggaacttcgaggaagtcgtcgataagggcgcctccgcccagtccttcatcgagcgcatgaccaacttcgataagaacctccctaacgagaaggtcctccctaagcactccctgctctacgagtacttcaccgtctacaacgagctgaccaaggtcaagtacgtcaccgagggtatgcgcaagcctgccttcctgtccggcgagcagaagaaggccatcgtcgatctgctgttcaagaccaaccgcaaggtcaccgtcaagcagctcaaagaggattacttcaagaaaatcgagtgcttcgattccgtcgagatcagcggcgtcgaggaccgcttcaacgcctccctcggaacctaccatgatctcctcaagattatcaaggataaggatttcctcgacaacgaggaaaacgaggacatccttgaggacatcgtcctcaccctcaccctcttcgaggaccgcgaaatgatcgaggaacgcctcaagacctacgcccatctcttcgatgataaggtcatgaagcagctcaagcgccgtcgctacaccggctggggtcgcctctcccgcaagctcatcaacggcatccgcgataagcagtccggcaagactatcctcgatttcctcaagtccgatggcttcgccaaccgcaacttcatgcagctcatccatgatgattccctcaccttcaaagaggacatccagaaggcccaggtcagcggccagggcgattccctccatgagcatatcgccaacctcgccggctcccctgccatcaagaagggcatcctccagaccgtcaaggtcgtcgatgagctggtcaaggtcatgggccgccataagcctgagaacatcgtcatcgagatggcccgcgagaaccagaccacccagaagggccagaagaactcccgcgagcgcatgaagcgcatcgaggaaggcatcaaagagctgggcagccaaatcctcaaagagcatcctgtcgagaacacccagctccagaacgagaagctctacctctactacctccagaacggccgcgatatgtacgtcgatcaagagctggacatcaaccgcctctccgattacgatgtcgatgctatcgtccctcagtccttcctgaaggatgattccatcgataacaaggtcctcacccgctccgataagaaccgcggcaagtccgataacgtcccttccgaagaggtcgtcaagaagatgaagaactactggcgccagctcctcaacgccaagctcatcacccagcgcaagttcgataacctcaccaaggccgagcgcggtggcctctccgagctggataaggccggcttcatcaagcgccagctcgtcgaaacccgccagatcaccaagcacgtcgcccaaatcctcgattcccgcatgaacaccaagtacgatgagaacgataagctcatccgcgaagtcaaggtcatcaccctcaagtccaagctcgtcagcgatttccgcaaggatttccagttctacaaggtccgcgagatcaacaactaccatcatgcccatgatgcctacctcaacgccgtcgtcggcaccgccctcatcaagaagtaccccaagctcgaatccgagttcgtctacggtgattacaaggtctacgatgtccgcaagatgatcgccaagtccgagcaagagatcggcaaggctaccgccaagtacttcttctactccaacatcatgaatttcttcaagaccgaaatcaccctcgccaacggcgaaatccgcaagcgccctctcatcgagactaacggcgagactggcgagatcgtctgggataagggccgcgatttcgccaccgtccgcaaggtcctctccatgcctcaggtcaacatcgtcaagaaaaccgaggtccagaccggcggcttctccaaagagtccatcctccccaagcgcaactccgataagctgatcgcccgcaagaaggattgggaccctaagaagtacggcggcttcgattcccctaccgtcgcctactccgtcctcgtcgtcgccaaggtcgagaagggcaagtccaagaagctcaagtccgtcaaagagctgctcggcatcactattatggaacgctccagcttcgagaagaaccctatcgatttccttgaggccaagggctacaaagaggtcaagaaggacctcatcatcaagctccccaagtactccctgttcgagcttgagaacggccgcaagcgcatgctcgcctccgccggtgagcttcagaagggcaacgagctggccctgccttccaagtacgtcaacttcctctacctcgcctcccattacgagaagctcaagggctcccctgaggataacgagcagaagcagctgttcgtcgagcagcataagcactacctcgatgagatcatcgagcagatcagcgagttctccaagcgcgtcatcctcgccgatgccaacctcgataaggtcctgtccgcctacaacaagcaccgcgataagcctatccgcgagcaggccgagaacatcatccatctcttcaccctcaccaacctcggtgcccctgccgccttcaagtacttcgataccaccatcgatcgcaagcgctacacctccaccaaagaggtcctggacgccaccctcatccatcagtccatcaccggcctctacgaaacccgcatcgatctctcccagctcggcggcgaccctccgaggaaacgtgccaaaacagaagatgagagcgctggaggaggtggaagcggaggaggaggaagcggaggaggaggtagcggacctaagaaaaagaggaaggtggcggccgcttaa

SEQ ID NO: 39 - DNA, 32 bp, Artificial sequence
  Primer
cgagatctgaacttaacgttactgaaatcatc

SEQ ID NO: 40 - DNA, 273 bp, Artificial sequence
  human zinc finger protein 10 after codon optimization
atggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatga

SEQ ID NO: 41 - DNA, 38 bp, Artificial sequence
  Primer
gctagcgttaacactagtcacctctaaacaagtgtacc

SEQ ID NO: 42 - DNA, 567 bp, Myceliophthora thermophila
  sgRNA expression frame vector U6p-2312390-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcgggctcagcacaaagtctgggttttagagctagaaatagcaagttaaaataaggctagtccgttatcaacttgaaaaagtggcaccgagtcggtgcttttttt

SEQ ID NO: 43 - DNA, 31 bp, Artificial sequence
  Primer
gcagatctcatgtaccttgacgtcctccgag

SEQ ID NO: 44 - DNA, 33 bp, Artificial sequence
  Primer
aaacactagttctgaagaacgaaactggcgact

SEQ ID NO: 45 - DNA, 40 bp, Artificial sequence
  Primer
tttaaacgatatcgaattcgagcatacaatcaactatctc

SEQ ID NO: 46 - DNA, 39 bp, Artificial sequence
  Primer
gatttcagtaacgttaagtgctttaaaatttgtatacac

SEQ ID NO: 47 - DNA, 43 bp, Artificial sequence
  Primer
ccacatcacagaaatcaaaactagtatgttcctgggcagccgc

SEQ ID NO: 48 - DNA, 35 bp, Artificial sequence
  Primer
tttctgggatagacataccgagaacggcgaccttg

SEQ ID NO: 49 - DNA, 29 bp, Artificial sequence
  Primer
cgttctcggtatgtctatcccagaaactc

SEQ ID NO: 50 - DNA, 50 bp, Artificial sequence
  Primer
tgttaataacccacgcgtgggcggatccttatttagaagtgtcaacaacg

SEQ ID NO: 51 - DNA, 38 bp, Artificial sequence
  Primer
agccatggagaggtttagcacgaacctctctgaaggag

SEQ ID NO: 52 - DNA, 19 bp, Artificial sequence
  Primer
gtatgatgggtcagttcag

SEQ ID NO: 53 - DNA, 27 bp, Artificial sequence
  Primer
cagccgcatccagacccgcgccttctc

SEQ ID NO: 54 - DNA, 18 bp, Artificial sequence
  Primer
atgggtctcttctcgaaa

SEQ ID NO: 55 - DNA, 18 bp, Artificial sequence
  Primer
ctaaacctctccatggct

SEQ ID NO: 56 - DNA, 38 bp, Artificial sequence
  Primer
aagaggagtccaccatggtacttgcatcgtcccaaagc

SEQ ID NO: 57 - DNA, 38 bp, Artificial sequence
  Primer
tttcgagaagagacccattttgatttctgtgatgtggg

SEQ ID NO: 58 - DNA, 37 bp, Artificial sequence
  Primer
tcacagaaatcaaaactagaatgtcgtctcacggctc

SEQ ID NO: 59 - DNA, 46 bp, Artificial sequence
  Primer
ttaagtggatccgaattcgatatcctaagcaacgatagcttcggac

SEQ ID NO: 60 - DNA, 38 bp, Artificial sequence
  Primer
aagaggagtccaccatggtacaggatattgataacacc

SEQ ID NO: 61 - DNA, 38 bp, Artificial sequence
  Primer
cgatctctagaagtcctcgtcccgagccaaacacttgg

SEQ ID NO: 62 - DNA, 38 bp, Artificial sequence
  Primer
caggtacacttgtttagaggccgctggacaggttcgtg

SEQ ID NO: 63 - DNA, 38 bp, Artificial sequence
  Primer
gctagcgttaacactagtcagcccatctgttgttaacc

SEQ ID NO: 64 - DNA, 37 bp, Artificial sequence
  Primer
acgaggacttctagagatcgttgcatcgtcccaaagc

SEQ ID NO: 65 - DNA, 20 bp, Artificial sequence
  Primer
cctctaaacaagtgtacctg

SEQ ID NO: 66 - DNA, 19 bp, Artificial sequence
  Primer
tcaagtccagcatagcgac

SEQ ID NO: 67 - DNA, 20 bp, Artificial sequence
  Primer
cctctaaacaagtgtacctg

SEQ ID NO: 68 - DNA, 38 bp, Artificial sequence
  Primer
aagaggagtccaccatggtacgtcaggtgtctgttctc

SEQ ID NO: 69 - DNA, 37 bp, Artificial sequence
  Primer
gtcgctatgctggacttgagtacgcagtcgtcacgcc

SEQ ID NO: 70 - DNA, 38 bp, Artificial sequence
  Primer
caggtacacttgtttagaggtattccgggacgtagtgc

SEQ ID NO: 71 - DNA, 38 bp, Artificial sequence
  Primer
gctagcgttaacactagtcactccaactcgctgaggag

SEQ ID NO: 72 - DNA, 31 bp, Artificial sequence
  Primer
ttatttagaagtgtcaacaacgtatctacca

SEQ ID NO: 73 - DNA, 30 bp, Artificial sequence
  Primer
ccggcgcatgatgtctgaaattactttggg

SEQ ID NO: 74 - DNA, 45 bp, Artificial sequence; identical to SEQ ID NO: 25
  Primer

SEQ ID NO: 75 - DNA, 44 bp, Artificial sequence
  Primer
ccacatcacagaaatcaaaactagtatgttggcttctcgtcagc

SEQ ID NO: 76 - DNA, 20 bp, Artificial sequence
  Primer
agtttatgtggaaaatggca

SEQ ID NO: 77 - DNA, 38 bp, Artificial sequence
  Primer
ctcgtagtcgccgacggactatccgttgggcggagcat

SEQ ID NO: 78 - DNA, 36 bp, Artificial sequence
  Primer
ccaacggatagtccgtcggcgactacgaggactgcg

SEQ ID NO: 79 - DNA, 20 bp, Artificial sequence
  Primer
tacgtgccgaccctcgagac

SEQ ID NO: 80 - DNA, 25 bp, Artificial sequence
  Primer
tcctccgaggttcgacatcagggtt

SEQ ID NO: 81 - DNA, 24 bp, Artificial sequence
  Primer
ctctaaacaagtgtacctgtgcat

SEQ ID NO: 82 - DNA, 20 bp, Artificial sequence
  Primer
agtttatgtggaaaatggca

SEQ ID NO: 83 - DNA, 20 bp, Artificial sequence
  Primer
tacgtgccgaccctcgagac

SEQ ID NO: 84 - DNA, 31 bp, Artificial sequence
  Primer
acggatagtcatattgaaggagcactttttg

SEQ ID NO: 85 - DNA, 29 bp, Artificial sequence
  Primer
gtcgccgacggatcactagtacaggattc

SEQ ID NO: 86 - DNA, 22 bp, Artificial sequence
  Primer
cggattcaagatgcctcagcca

SEQ ID NO: 87 - DNA, 20 bp, Artificial sequence
  Primer
gaccgggttggtggccacga

SEQ ID NO: 88 - DNA, 21 bp, Artificial sequence
  Primer
cctgactgtataagagaaatg

SEQ ID NO: 89 - DNA, 40 bp, Artificial sequence
  Primer
agcgaccggagtaagctcccgccgtggcaatttcccgaac

SEQ ID NO: 90 - DNA, 37 bp, Artificial sequence
  Primer
aaattgccacggcgggagcttactccggtcgctgtca

SEQ ID NO: 91 - DNA, 23 bp, Artificial sequence
  Primer
ccaatgaagcgttccttacggct

SEQ ID NO: 92 - DNA, 22 bp, Artificial sequence
  Primer
aggatcggtggagtgaagttcg

SEQ ID NO: 93 - DNA, 40 bp, Artificial sequence
  Primer
acggagcaggtaaccaagtcgaggaaagaaagaaaagaag

SEQ ID NO: 94 - DNA, 40 bp, Artificial sequence
  Primer
gacttggttacctgctccgtgttttagagctagaaatagc

SEQ ID NO: 95 - DNA, 23 bp, Artificial sequence
  Primer
aaaaaaagcaccgactcggtgcc

SEQ ID NO: 96 - DNA, 23 bp, Artificial sequence
  Primer
tgactgaccgtgatgatttccag

SEQ ID NO: 97 - DNA, 22 bp, Artificial sequence
  Primer
agcctaacgtcgagaccggcgt

SEQ ID NO: 98 - DNA, 23 bp, Artificial sequence
  Primer
gggtggtgtgcatggggccgtct

SEQ ID NO: 99 - DNA, 39 bp, Artificial sequence
  Primer
aaagtgctccttcaatatgcagcagggtcagccgggtct

SEQ ID NO: 100 - DNA, 40 bp, Artificial sequence
  Primer
cggctgaccctgctgcatattgaaggagcactttttgggc

SEQ ID NO: 101 - DNA, 35 bp, Artificial sequence
  Primer
ggtcatgccgggcttttaagaaactttattgccaa

SEQ ID NO: 102 - DNA, 41 bp, Artificial sequence
  Primer
ggcaataaagtttcttaaaagcccggcatgacccgtgatga

SEQ ID NO: 103 - DNA, 25 bp, Artificial sequence
  Primer
cgtactgaagctcttcctctatcct

SEQ ID NO: 104 - DNA, 39 bp, Artificial sequence
  Primer
ttggcgggcaggtagccgaggaaagaaagaaaagaagag

SEQ ID NO: 105 - DNA, 40 bp, Artificial sequence
  Primer
ctacctgcccgccaacgagttttagagctagaaatagcaa

SEQ ID NO: 106 - DNA, 22 bp, Artificial sequence
  Primer
aggatcggtggagtgaagttcg

SEQ ID NO: 107 - DNA, 23 bp, Artificial sequence
  Primer
aaaaaaagcaccgactcggtgcc

SEQ ID NO: 108 - DNA, 28 bp, Artificial sequence
  Primer
tttcagacatcatgcgccggagaccaag

SEQ ID NO: 109 - DNA, 24 bp, Artificial sequence
  Primer
atgtctgaaattactttgggtaaa

SEQ ID NO: 110 - DNA, 22 bp, Artificial sequence
  Primer
tgggacggcgagagcgacgggt

SEQ ID NO: 111 - DNA, 31 bp, Artificial sequence
  Primer
gcgtcgagaaggcagagtctgtggatcgtgc

SEQ ID NO: 112 - DNA, 28 bp, Artificial sequence
  Primer
agactctgccttctcgacgccgtcaggg

SEQ ID NO: 113 - DNA, 23 bp, Artificial sequence
  Primer
tagcgccagttgcgtgccgaggt

SEQ ID NO: 114 - DNA, 40 bp, Artificial sequence
  Primer
cggggattgtgacattctccgaggaaagaaagaaaagaag

SEQ ID NO: 115 - DNA, 40 bp, Artificial sequence
  Primer
ggagaatgtcacaatccccggttttagagctagaaatagc

SEQ ID NO: 116 - DNA, 22 bp, Artificial sequence
  Primer
aggatcggtggagtgaagttcg

SEQ ID NO: 117 - DNA, 23 bp, Artificial sequence
  Primer
aaaaaaagcaccgactcggtgcc

SEQ ID NO: 118 - DNA, 21 bp, Artificial sequence
  Primer
cgctctcattccccctcgcag

SEQ ID NO: 119 - DNA, 22 bp, Artificial sequence
  Primer
atatactcgcaaatcagctcga

SEQ ID NO: 120 - DNA, 62 bp, Artificial sequence
  Primer
aagcttatggaccccaagaagaaacgcaaggttgataagaagtactccatcggcctcgccat

SEQ ID NO: 121 - DNA, 32 bp, Artificial sequence
  Primer
tgagggacgatagcatcgacatcgtaatcgga

SEQ ID NO: 122 - DNA, 32 bp, Artificial sequence
  Primer
cgatgtcgatgctatcgtccctcagtccttcc

SEQ ID NO: 123 - DNA, 23 bp, Artificial sequence
  Primer
agcggccgccaccttcctctttt

SEQ ID NO: 124 - DNA, 33 bp, Artificial sequence
  Primer
gggtttaaacatggaccccaagaagaaacgcaa

SEQ ID NO: 125 - DNA, 28 bp, Artificial sequence
  Primer
tagcatccatagcggccgccaccttcct

SEQ ID NO: 126 - DNA, 29 bp, Artificial sequence
  Primer
cggccgctatggatgctaagtcactaact

SEQ ID NO: 127 - DNA, 34 bp, Artificial sequence
  Primer
ccttaattaatgcagtctctgaatcaggatgggt

SEQ ID NO: 128 - DNA, 29 bp, Artificial sequence
  Primer
ccggatctcggatcactagtacaggattc

SEQ ID NO: 129 - DNA, 29 bp, Artificial sequence
  Primer
actagtgatccgagatccggggtgacacc

SEQ ID NO: 130 - DNA, 22 bp, Artificial sequence
  Primer
cagtcgcccacggcccagatgt

SEQ ID NO: 131 - DNA, 22 bp, Artificial sequence
  Primer
aggatcggtggagtgaagttcg

SEQ ID NO: 132 - DNA, 37 bp, Artificial sequence
  Primer
cggcgctctggatgatcgaggaaagaaagaaaagaag

SEQ ID NO: 133 - DNA, 35 bp, Artificial sequence
  Primer
catccagagcgccggcagttttagagctagaaata

SEQ ID NO: 134 - DNA, 23 bp, Artificial sequence
  Primer
aaaaaaagcaccgactcggtgcc

SEQ ID NO: 135 - DNA, 22 bp, Artificial sequence
  Primer
tcaagtgtcatgatctcgcgtg

SEQ ID NO: 136 - DNA, 28 bp, Artificial sequence
  Primer
tcatcttctgggtttggatgaatggatg

SEQ ID NO: 137 - DNA, 31 bp, Artificial sequence
  Primer
catccaaacccagaagatgatattgaaggag

SEQ ID NO: 138 - DNA, 32 bp, Artificial sequence
  Primer
cgcaacttgagaaagaaggattacctctaaac

SEQ ID NO: 139 - DNA, 28 bp, Artificial sequence
  Primer
tccttctttctcaagttgcggggcgcgt

SEQ ID NO: 140 - DNA, 23 bp, Artificial sequence
  Primer
aggactattatcatcctggggga

SEQ ID NO: 141 - DNA, 28 bp, Artificial sequence
  Primer
ccggcgcatgatggccaccaccaagacc

SEQ ID NO: 142 - DNA, 38 bp, Artificial sequence
  Primer
gatgggcgtcttgagctcgaggaaagaaagaaaagaag

SEQ ID NO: 143 - DNA, 39 bp, Artificial sequence
  Primer
gctcaagacgcccatcacgttttagagctagaaatagca

SEQ ID NO: 144 - DNA, 567 bp, Myceliophthora thermophila
  sgRNA expression plasmids U6p-nde1-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcgattggcttctatcagggctgttttagagctagaaatagcaagttaaaataaggctagtccgttatcaacttgaaaaagtggcaccgagtcggtgcttttttt

SEQ ID NO: 145 - DNA, 2266 bp, Myceliophthora thermophila
  donor DNA fragments donor-nde1
cgggctcagccggaacttgctacctctgtaaactttcgactgtggattttggcgctcgcttctgttcgtggacgccccatgacatcagtgaacaatttcataaacccccaccatgttcagacttttgtcaaacctcttctgtcctttggttctttcgtttattattttttttttccgtaaaatgcatctttggggacacaggtagtttacacacagcctcgggttgaatatgtgaatgagcctcgtgatatctatccaggagttggtagcggaaagcagcgttgactgaaccgggatattaagccgccgatatcacctttatctgcctcgaagatgcccctttacggtcctaacatgacttggtgccgccaacttgtaggccggacaaacgctccaagacgatgctttagtatcgctaaaagtaagtactttcggcttcgaaccaagtgcgtccgtctcgttaaccgatcccaccagacaatggcacacacaaggagcgggtagtgatccttggctcaggttgggcagggtatggtttcgctcgcactcttgacccagccaagtacgagcgcattatcatctcgccacgctcctactttgtcttcaccccgttgctagccatattgaaggagcactttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtgaacgacccggtcataccttcttaagttcgcccttcctccctttatttcagattcaatctgacttacctattctacccaagcaaagcttcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaagattgaatcctgtactagtgatccggtcatccccgctccttctccctcatcatcatcagaaaccggcgggccgggcaaggcacccaagggcgacctcatcgagatcccttacgacaagcttgtgatcgcttgcggcgcgtacagccaaacctttggcatcgagggagtccgcgaatatgcacacttcctccgcgacatcggcgacgcccggcgcatccgccttcgcgtcctctccctctttgagcagtgcgcctatccccgcgggtccgaccacctcagcgacgacgacaagcgccagctcctccacttcgccatcgtgggcggcggaccgaccggcatcgaattcgccgccgagctccacgaccttatccgcgatgacctcgcccccatctaccccgagctcgccccgctggtcagcatcacggtctacgacgtggcccccaaggttcttccaatgttcgaccgggcgctcgcaaagtacgccatggacaccttctcacgacaaaacatcaaggtcaagaccgagcaccatctcgagcgcctccgcctggccgacggggagctcggccgccgccgcggagtcctcaagatcaagatcaaggagtacggcgacgaggaagtcggcgcgggcctggtcgtctggagcacaggcctcatgccaaaccccttgatc

SEQ ID NO: 146 - DNA, 567 bp, Myceliophthora thermophila
  sgRNA expression plasmids U6p-nde2-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcgaccatcctccgccagaagagttttagagctagaaatagcaagttaaaataaggctagtccgttatcaacttgaaaaagtggcaccgagtcggtgcttttttt

SEQ ID NO: 147 - DNA, 2341 bp, Myceliophthora thermophila
  donor DNA fragments donor-nde2
tcctcctccctctctcaggcgccctccgccgtgtcctacctcgcgcgctcgtcaattgagccaatggcatccacgtcgagagctctgagcaagctcgcggtgccccaaacgggcgccgttatgcttcagccgtcttcgtccgcgcccagactgctcctccagacatcgcgcaggaccgtaggcgcgaggcccgtcttcggcagctcacaagggcccgctctccggcagttccggcgcggcgtcaccagcgacgccgcccccgtcccgccgcccaagaagcggcgattccgcaagctgcggtgggcatggcggttcacatacctctccgccatcgccagcatcgcctacatcggctacggcgtctaccaggacaggcacccggagccccaggtcgagcccgacccgtccaagaagaccttggtgattctcggtaagttttggccccgtcccgtcatcccgtcacaccgcactgttaaagaagcttgttccgcggttggggggaatgtagaaagggaaaaaaaaaaagagagagagagagagagagaaaatattctccggggctaacaagcatgtgccgcaggaaccggctggggctctgtctcgctgctcaagcgcctcgacacggagaactacaacgtcatcgtcatctccccgcgcaactatattgaaggagcactttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtgaacgacccggtcataccttcttaagttcgcccttcctccctttatttcagattcaatctgacttacctattctacccaagcaaagcttcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaagattgaatcctgtactagtgatccgagatccggggtgacaccaccgagaccgaggtgccgtacgacatgctggtggtcggcgtgggcgccgagaacgccacgttcggcatccccggcgtgcgcgagcactcgtgcttcctcaaggagatcggcgacgcccaaaagatccgcaagcgcatcatggactgcgtcgagacggcggcgttcaaggaccagtcgcccgaggagatcgaccggctcctgcacatggtggtcgtcgggggcggcccgaccggcgtcgagttcgccggcgagctgcaggacttcttcgaggaggacatcaagaagctggtgcccgagatcagcgaccgcttccgggtgacgctcatcgaggcgctgcctaacgtgctgcccatgttctccaagcagctgatcgagtacaccgagagcaccttcaaggaggagaagatcaacatccacaccaagaccgtggtcaagaaggtgacggacaagacggtcgaggccgaggtgacgcgcccggacggcaagaaggagacggtcgtcttcccctacggcctgctcgtctgggccaccggtaacgccgtccgccccgtcgtccgggacctgatgtcgcgcatcccggcccaggccggctcgcgccgcggcctcgccgtcaacgagtacctcgtcgtccagggcacgcgcgacatctgggccgtgggcgactg

SEQ ID NO: 148 - DNA, 22 bp, Artificial sequence
  Primer
aggatcggtggagtgaagttcg

SEQ ID NO: 149 - DNA, 37 bp, Artificial sequence
  Primer
ctgatagaagccaatcgaggaaagaaagaaaagaaga

SEQ ID NO: 150 - DNA, 40 bp, Artificial sequence
  Primer
gattggcttctatcagggctgttttagagctagaaatagc

SEQ ID NO: 151 - DNA, 23 bp, Artificial sequence
  Primer
aaaaaaagcaccgactcggtgcc

SEQ ID NO: 152 - DNA, 24 bp, Artificial sequence
  Primer
cgggctcagccggaacttgctacc

SEQ ID NO: 153 - DNA, 29 bp, Artificial sequence
  Primer
ccttcaatatggctagcaacggggtgaag

SEQ ID NO: 154 - DNA, 31 bp, Artificial sequence
  Primer
gttgctagccatattgaaggagcactttttg

SEQ ID NO: 155 - DNA, 29 bp, Artificial sequence
  Primer
gggatgaccggatcactagtacaggattc

SEQ ID NO: 156 - DNA, 28 bp, Artificial sequence
  Primer
actagtgatccggtcatccccgctcctt

SEQ ID NO: 157 - DNA, 21 bp, Artificial sequence
  Primer
gatcaaggggtttggcatgag

SEQ ID NO: 158 - DNA, 43 bp, Artificial sequence
  Primer
gatttcagtaacgttaagtggatcctcaggcgccgtaggcctt

SEQ ID NO: 159 - DNA, 37 bp, Artificial sequence
  Primer
tctggcggaggatggtcgaggaaagaaagaaaagaag

SEQ ID NO: 160 - DNA, 40 bp, Artificial sequence
  Primer
accatcctccgccagaagagttttagagctagaaatagca

SEQ ID NO: 161 - DNA, 44 bp, Artificial sequence; identical to SEQ ID NO: 75
  Primer

SEQ ID NO: 162 - DNA, 23 bp, Artificial sequence
  Primer
tcctcctccctctctcaggcgcc

SEQ ID NO: 163 - DNA, 29 bp, Artificial sequence
  Primer
ccttcaatatagttgcgcggggagatgac

SEQ ID NO: 164 - DNA, 31 bp, Artificial sequence
  Primer
ccgcgcaactatattgaaggagcactttttg

SEQ ID NO: 165 - DNA, 1332 bp, Myceliophthora thermophila
  The nucleotide sequences of phosphofructokinase2 (PFK2, Mycth_71484, Gene ID 11510101)
atggctccaaagattaacggcactggcgtcatgcccgaggacacgcgcatctgcgttgtcatggtcggccttccggctcggggcaagagttacatagcccaaagagcgcaacgctatctcaagtggctgtcaatcccggcgaagaccttcaacgtgggcaactaccgccggaaagatgcgccacatccatcagccgacttcttcgacaccaacaacgccgaaggcgagagacagcgccgcgcagctgccaacgccgccgtgacagacatgatccagtggttcaaaaccggcggtgttgtcggaatccttgacgctacaaacagcaccaaagaacggcggaaatgggtactcgagcgactctctaaagaggggatcgaggtcctctttgtcgagtccaagtgcgacgacgaggaactgataatggccaacatccgcgatgtcaaaaccacttccccggattaccagggtcaggaccctgagcaggctgcccttgacttccgcgaacgcatccgccattacgagaaggtctacaagtcgatcaacgacgataatgatgaagaccacttgacgtacctcaagataatgaatgtgggcaagaaggtcttcatcaaccgcattcaggactacctacagagccgcgtcgtctacttcctcatgaacctgcatatccggccccgctcggtttggctgtctcgccatggcgagtctgaatacaatttggaagggcggattggcggtgattcattgctctctcaccgcggcgaagagtacgcccgcaaattgcccgaattggttcgccagtctgttggtagcgaccgaccattaacggtctggacgtcgacgctgaaacggaccatcgccacagcccggcacttgccaaaacactacaaccaactgcagtggaaggcgctggatgagctagacgctggtgtctgcgacgggttgacgtaccaggagattgcagaccagtatccggaggactttcaggcgcgcgacgaagataaatacaactaccgatatcgcggcggcgagagctaccgcgacgtcgtcattcgcctcgaacccatcatcatggaactcgagcgcagcgaaaacatcctcatcatctcgcaccaggccgtcatccgctgcatctacgcctacttcatgcaaaagcctcaagaggaaagcccgtgggtgcctgtaccgctgcacaccctaatcaagctcacgccccgcgcctacgacaccctggtggagaagtacgatgcgaaaatcaaggccgtgagcacctggcgcggcaaggggagcaccgccaaacacgaagatccaaccccagaaggcggcatttga

SEQ ID NO: 166 - protein, 443 aa, Myceliophthora thermophila
  phosphofructokinase2 (PFK2, Mycth_71484, Gene ID 11510101)
MAPKINGTGVMPEDTRICVVMVGLPARGKSYIAQRAQRYLKWLSIPAKTFNVGNYRRKDAPHPSADFFDTNNAEGERQRRAAANAAVTDMIQWFKTGGVVGILDATNSTKERRKWVLERLSKEGIEVLFVESKCDDEELIMANIRDVKTTSPDYQGQDPEQAALDFRERIRHYEKVYKSINDDNDEDHLTYLKIMNVGKKVFINRIQDYLQSRVVYFLMNLHIRPRSVWLSRHGESEYNLEGRIGGDSLLSHRGEEYARKLPELVRQSVGSDRPLTVWTSTLKRTIATARHLPKHYNQLQWKALDELDAGVCDGLTYQEIADQYPEDFQARDEDKYNYRYRGGESYRDVVIRLEPIIMELERSENILIISHQAVIRCIYAYFMQKPQEESPWVPVPLHTLIKLTPRAYDTLVEKYDAKIKAVSTWRGKGSTAKHEDPTPEGGI

SEQ ID NO: 167 - DNA, 567 bp, Myceliophthora thermophila
  sgRNA expression frame U6p-pfk2-sgRNA
aggatcggtggagtgaagttcggaatcgaggttcggcgatgggtcgtaagcatggcgacttcgaacttacttgcactggcaagcgttgccagaacggcgagaaaaagaagggtaagcgatattcgcgtcatgatggactgttccttttggaacagtagttgttgtgggaagactatgtcacacttgcccacctgcaaggccagggtcgtggtcgaacgagaccagcctcggcgctgctgggagctcaagatgggcacgtttgattcgttagacgtcaacaaggctggagttcctagtgacagccaaaggcacagccacattaagtggcgctttatctgtccactaaggttcaattgtggctttgagccgcgcagtgtgcagtcgtgcattggccacctagctagcagtatttaagatcctcttctctcccgagatcttcctcctcttcttttctttctttcctcggagattgcagaccagtatcgttttagagctagaaatagcaagttaaaataaggctagtccgttatcaacttgaaaaagtggcaccgagtcggtgcttttttt

SEQ ID NO: 168 - DNA, 2954 bp, Myceliophthora thermophila
  donor DNA fragment donor-pfk2
ggctcgggtcggttaatgggaactgctcgcttcccggtttcagccggccgagtatctagaaggcactctgactggactggaagttgtgcatatttggaaatatgaggagcaattattgattcggcagagggctcgcaatcagacagagcgtggggggaggcgatttgtggttggcattgctagatggcggtcagacgggtccactggccaaaggctgccagctttcgttcctgcagggagaaagcgaaaacccatcgcgtcacgggcgctcgaacccagtcgaacccagcctgagcgcccccacttttagtctgcctcttcgggttcgtccggggaagaacggctggagccaaaaacccgctgttggccaggctctggctgaacttggcggcgccatgatgcccaggaaccaggatcttcgcccgctggcccgccttacttgccgtccctgggaatcccgatgcaacaagacaagtgatgtagctcgatgctaccgaggtgcagaaagcaaaattgcggttgctcttcttcctttcgacctaaaatttccacaccgacgacgtcgccgtcgtccctccgagattttgcagccgttggagcccattgtttcattcccgccgtgcctcgctgcttcgacgcgcggcgacagcgccaaaggtgtgacgtcggaagtgggagagctcccctctcaatagcccactatcgccaaccgccttctcggcccgcagccggtggcccacgaagcgctcacgccatcaacatctacgcacgctccccgctgacatctccggctctgcgctgttcctcggccccaatcaggctgctacatgtctcgatcacctatcctgccctcctccgcctccagaacgttgctacagcggctgtcgctcagcaaagatattgaaggagcactttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtgaacgacccggtcataccttcttaagttcgcccttcctccctttatttcagattcaatctgacttacctattctacccaagcaaagcttcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgacatctgcctcacggggtgaacacgggaggcgtgccaacgtgatttgccgtattgtctgttctcttctctcagcatatctagacacgatgtttcgtttcctatttttggcgacaggctgtgccttgcatttctgggataacatcatatgagaagtccgtggttgaaactgggcggaaacctctgggccggcgttttgcggttgggtgtcagcgttggcttttgctcacggactgagcttttgagtccgacaaggaaatgatacgctactggtaccatctgggagcggttacttggctggtttagacggcaaaggaaaacaaaaaacgggccaactggacgcatggaatggacctaaatcgacggctcgcacggaagcacgtatggacgctgcgtgctctgccaccgggcagtggcattatctacatgcccaattgggccaacatttggcattataaagactaagcattgccgcttttatctcttgaccaagtttccgatgattctgtcacataccatccacgtgtcgaggaaacagcctttggtcaatgggagtgtgggcgccatctccttattgcgtttcgttccttgataccgcgacaaagtcttctgtccgtggcctaactgtgcagggaggattcaacttcccaaatatttgaaacgcatgctttctacaggctagcattgggcattcggctccgtgtggatgtctcagaagagaaaacatggtgctgctcacattatacaatgaatgccagagagtatcaggggcaatgacagtgcttaagatgctaggctaaagcgtccgttttgtcgtgcaaccatccgccgagattgacacacttgcttctgcaccaaattgccccttctagcggtaaacgctgaccatgatagcttgagagcaatacatggcaaaggcacagcagcgcctatcaataatgttggcgggggatgaaacgcatctgggcttgaccaacgtcgaagatgaccagcgcgttttgcatgaatgcgtccccaagaatatatgggcccgagcctccatccgcaatggccgtcatgcaaagccccgtcaccggatcaaccaaggtacgataaaggaggtccaaagggctgatgttgaactttacgccattga

SEQ ID NO: 169 - DNA, 34 bp, Artificial sequence
  Primer
tcttggtggtggccatcatgcgccggagaccaag

SEQ ID NO: 170 - DNA, 1332 bp, Myceliophthora thermophila
  The nucleotide sequences after mutation of phosphofructokinase 2
atggctccaaagattaacggcactggcgtcatgcccgaggacacgcgcatctgcgttgtcatggtcggccttccggctcggggcaagagttacatagcccaaagagcgcaacgctatctcaagtggctgtcaatcccggcgaagaccttcaacgtgggcaactaccgccggaaagatgcgccacatccatcagccgacttcttcgacaccaacaacgccgaaggcgagagacagcgccgcgcagctgccaacgccgccgtgacagacatgatccagtggttcaaaaccggcggtgttgtcggaatccttgacgctacaaacagcaccaaagaacggcggaaatgggtactcgagcgactctctaaagaggggatcgaggtcctctttgtcgagtccaagtgcgacgacgaggaactgataatggccaacatccgcgatgtcaaaaccacttccccggattaccagggtcaggaccctgagcaggctgcccttgacttccgcgaacgcatccgccattacgagaaggtctacaagtcgatcaacgacgataatgatgaagaccacttgacgtacctcaagataatgaatgtgggcaagaaggtcttcatcaaccgcattcaggactacctacagagccgcgtcgtctacttcctcatgaacctgcatatccggccccgctcggtttggctgtctcgcggcggcgagtctgaatacaatttggaagggcggattggcggtgattcattgctctctcaccgcggcgaagagtacgcccgcaaattgcccgaattggttcgccagtctgttggtagcgaccgaccattaacggtctggacgtcgacgctgaaacggaccatcgccacagcccggcacttgccaaaacactacaaccaactgcagtggaaggcgctggatggcctagacgctggtgtctgcgacgggttgacgtaccaggagattgcagaccagtatccggaggactttcaggcgcgcgacgaagataaatacaactaccgatatcgcggcggcgagagctaccgcgacgtcgtcattcgcctcgaacccatcatcatggaactcgagcgcagcgaaaacatcctcatcatctcgggccaggccgtcatccgctgcatctacgcctacttcatgcaaaagcctcaagaggaaagcccgtgggtgcctgtaccgctgcacaccctaatcaagctcacgccccgcgcctacgacaccctggtggagaagtacgatgcgaaaatcaaggccgtgagcacctggcgcggcaaggggagcaccgccaaacacgaagatccaaccccagaaggcggcatttga

SEQ ID NO: 171 - protein, 443 aa, Myceliophthora thermophila
  The amino acid sequences after mutation of phosphofructokinase 2
MAPKINGTGVMPEDTRICVVMVGLPARGKSYIAQRAQRYLKWLSIPAKTFNVGNYRRKDAPHPSADFFDTNNAEGERQRRAAANAAVTDMIQWFKTGGVVGILDATNSTKERRKWVLERLSKEGIEVLFVESKCDDEELIMANIRDVKTTSPDYQGQDPEQAALDFRERIRHYEKVYKSINDDNDEDHLTYLKIMNVGKKVFINRIQDYLQSRVVYFLMNLHIRPRSVWLSRGGESEYNLEGRIGGDSLLSHRGEEYARKLPELVRQSVGSDRPLTVWTSTLKRTIATARHLPKHYNQLQWKALDGLDAGVCDGLTYQEIADQYPEDFQARDEDKYNYRYRGGESYRDVVIRLEPIIMELERSENILIISGQAVIRCIYAYFMQKPQEESPWVPVPLHTLIKLTPRAYDTLVEKYDAKIKAVSTWRGKGSTAKHEDPTPEGGI

SEQ ID NO: 172 - DNA, 28 bp, Artificial sequence
  Primer
ctgtcggcagcgcgagccttggtctccg

SEQ ID NO: 173 - DNA, 22 bp, Artificial sequence
  Primer
atggctccaaagattaacggca

SEQ ID NO: 174 - DNA, 24 bp, Artificial sequence
  Primer
tcagactcgccgccgcgagacagc

SEQ ID NO: 175 - DNA, 24 bp, Artificial sequence
  Primer
gctgtctcgcggcggcgagtctga

SEQ ID NO: 176 - DNA, 24 bp, Artificial sequence
  Primer
cagcgtctaggccatccagcgcct

SEQ ID NO: 177 - DNA, 24 bp, Artificial sequence
  Primer
aggcgctggatggcctagacgctg

SEQ ID NO: 178 - DNA, 24 bp, Artificial sequence
  Primer
atgacggcctggcccgagatgatg

SEQ ID NO: 179 - DNA, 24 bp, Artificial sequence
  Primer
catcatctcgggccaggccgtcat

SEQ ID NO: 180 - DNA, 23 bp, Artificial sequence
  Primer
tcaaatgccgccttctggggttg

SEQ ID NO: 181 - DNA, 45 bp, Artificial sequence
  Primer
gccagtttcgttcttcagaactagtatggctccaaagattaacgg

SEQ ID NO: 182 - DNA, 43 bp, Artificial sequence
  Primer
gatttcagtaacgttaagtggatcctcaaatgccgccttctgg

SEQ ID NO: 183 - DNA, 22 bp, Artificial sequence
  Primer
aggatcggtggagtgaagttcg

SEQ ID NO: 184 - DNA, 40 bp, Artificial sequence
  Primer
atactggtctgcaatctccgaggaaagaaagaaaagaaga

SEQ ID NO: 185 - DNA, 42 bp, Artificial sequence
  Primer
agattgcagaccagtatcgttttagagctagaaatagcaagt

SEQ ID NO: 186 - DNA, 23 bp, Artificial sequence
  Primer
aaaaaaagcaccgactcggtgcc

SEQ ID NO: 187 - DNA, 43 bp, Artificial sequence
  Primer
tacacagtacacgaggacttctagaggctcgggtcggttaatg

SEQ ID NO: 188 - DNA, 28 bp, Artificial sequence
  Primer
ccttcaatatctttgctgagcgacagcc

SEQ ID NO: 189 - DNA, 33 bp, Artificial sequence
  Primer
ctcagcaaagatattgaaggagcactttttggg

SEQ ID NO: 190 - DNA, 28 bp, Artificial sequence
  Primer
gaggcagatgtcagatctcggtgacggg

SEQ ID NO: 191 - DNA, 28 bp, Artificial sequence
  Primer
cgagatctgacatctgcctcacggggtg

SEQ ID NO: 192 - DNA, 50 bp, Artificial sequence
  Primer
aatatcatcttctgtcgaggaattctcaatggcgtaaagttcaacatcag

SEQ ID NO: 193 - DNA, 27 bp, Artificial sequence
  Primer
catcatcgatacactggttcttgacaa

SEQ ID NO: 194 - DNA, 25 bp, Artificial sequence
  Primer
ctcagggtcctgaccctggtaatcc

SEQ ID NO: 195 - protein, 859 aa, Thermoanaerobacterium saccharolyticum
  acetaldehyde dehydrogenase adhE
MATTKTELDVQKQIDLLVSRAQEAQKKFMSYTQEQIDAIVKAMALAGVDKHVELAKMAYEETKMGVYEDKITKNLFATEYVYHDIKNEKTVGIINENIEENYMEVAEPIGVIAGVTPVTNPTSTTMFKCLISIKTRNPIIFSFHPKAIKCSIAAAKVMYEAALKAGAPEGCIGWIETPSIEATQLLMTHPGVSLILATGGAGMVKAAYSSGKPALGVGPGNVPCYIEKSANIKRAVSDLILSKTFDNGVICASEQAVIIDEEIADEVKKLMKEYGCYFLNKDEIKKLEKFAIDEQSCAMSPAVVGQPAAKIAEMAGFKVPEGTKILVAEYEGVGPKYPLSREKLSPILACYTVKDYNEGIKKCEEMTEFGGLGHSAVIHSENQNVINEFARRVRTGRLIVNSPSSQGAIGDIYNTNTPSLTLGCGSMGRNSTTDNVSVKNLLNIKRVVIRKDRMKWFKIPPKIYFESGSLQYLCKVKRKKAFIVTDPFMVKLGFVDKVTYQLDKANIEYEIFSEVEPDPSVDTVMNGVKIMNSYNPDLIIAVGGGSAIDAAKGMWLFYEYPDTEFETLRLKFADIRKRAFKFPELGKKALFIAIPTTSGTGSEVTAFAVITDKKRNIKYPLADYELTPDIAIIDPDLTKTVPPSVTADTGMDVLTHAIEAYVSVMASDYTDALAEKAIKIVFEYLPRAYKNGNDEEAREKMHNASCMAGMAFTNAFLGINHSMAHILGGKFHIPHGRANAILLPYVIRYNAEKPTKFVAFPQYEYPKAAERYAEIAKFLGLPASTVEEGVESLIEAIKNLMKELNIPLTLKDAGINKEQFEKEIEEMSDIAFNDQCTGTNPRMPLTKEIAEIYRKAYGA

SEQ ID NO: 196 - DNA, 2580 bp, Thermoanaerobacterium saccharolyticum
  the DNA sequence of acetaldehyde dehydrogenase adhE after codon optimization
atggccaccaccaagaccgagctcgacgtccagaagcagatcgacctcctcgtcagccgcgcccaggaggcccagaagaagttcatgagctacacccaggagcagatcgacgccatcgtcaaggccatggccctcgccggcgtcgacaagcacgtcgagctcgccaagatggcctacgaggagaccaagatgggcgtctacgaggacaagatcaccaagaacctcttcgccaccgagtacgtctaccacgacatcaagaacgagaagaccgtcggcatcatcaacgagaacatcgaggagaactacatggaggtcgccgagccgatcggcgtcatcgccggcgtcactcccgtaaccaacccgaccagcacaacaatgtttaaatgtctgatatctattaaaactcgaaaccctattatattctctttccacccgaaggcgattaaatgttcgattgccgcagcgaaggtcatgtatgaagctgctctgaaagcaggcgcaccggaaggctgcatcggctggatcgagactccctcaatcgaggccacccagctcttgatgacacatcccggagtttcgttgattctggctactggaggagcaggaatggtaaaggcagcgtattcttccggaaagccggcgctgggcgtggggccgggaaatgtcccgtgctacattgaaaagtcggccaacatcaaacgggcagtgagcgacctcatcctcagcaagaccttcgacaacggcgtcatctgcgcaagtgaacaggccgtcatcatcgacgaagaaattgccgatgaagtcaagaagcttatgaaggagtacgggtgctactttctgaataaggacgagatcaagaagttagagaaattcgcgatcgatgagcagtcatgtgcaatgtctccagcagtagtgggacagccggccgccaagatagcagaaatggctggcttcaaggtcccggagggcaccaagatcctcgtcgccgagtacgagggagttggacccaagtacccacttagtcgtgagaagctcagcccgatcctcgcctgctacaccgtcaaggactacaacgagggcatcaagaagtgcgaggagatgaccgagttcggcggattgggtcactccgcagttatacactccgaaaatcagaacgtaattaacgagtttgctcggcgagttaggacgggccgcttaattgtcaacagcccgagcagccagggcgccatcggcgacatctacaacaccaacacgccttcgctcaccctcggctgcgggtccatggggcgtaattcaaccacggacaatgtttcagttaagaatcttttgaatataaagcgcgtggtgatccgcaaggaccgcatgaagtggttcaagatcccgccgaagatctacttcgagagcggcagcctccagtacctctgcaaggtcaagcgcaagaaggccttcatcgtcaccgacccgttcatggtcaagctcggcttcgtcgacaaggtcacctaccagctcgacaaggccaacatcgagtacgagatcttcagcgaggtcgagccggacccgagcgtcgacaccgtcatgaacggcgtcaagatcatgaacagctacaatcctgatctgattatagccgtcggcggcggcagcgccatcgacgccgccaagggcatgtggctcttctatgaatatcccgatactgagttcgagaccctccgcctcaagttcgccgacatccgcaagcgcgccttcaagttcccggagctcggcaagaaggccctcttcatcgccatcccgaccaccagcggcaccggcagcgaggtcaccgccttcgccgtcatcaccgacaagaagcgcaacatcaaatatccgcttgctgattacgagctcacacccgatatcgccatcatcgatcccgatttgacgaagacagtgccgccgagcgtcaccgccgacaccggcatggacgtcctcacccacgccatcgaggcctatgtgagtgtaatggcaagcgactacaccgacgccctcgccgagaaggctattaagatcgtctttgaatacctcccgcgcgcctacaagaacggcaacgacgaggaggcccgcgagaagatgcacaacgccagctgcatggccggcatggccttcaccaacgccttcctcggcatcaaccacagcatggcccacatcctcggcggcaagttccacatcccgcacggccgcgccaacgccatcctcctcccgtacgtcatccgctacaacgccgagaagccgaccaagttcgtcgccttcccgcagtatgaatatccaaaggccgcggagcgctacgccgagatcgccaagttcctcggcctcccggccagcactgtagaggagggcgtcgagagcttaattgaagctattaagaatttgatgaaagagttaaacatcccgctcaccctcaaggacgccggcatcaacaaggagcagttcgagaaggagatcgaggagatgagcgacatcgccttcaacgaccagtgcaccggcaccaacccgcgcatgccgctcaccaaagagatagctgagatttatcgcaaggcctacggcgcctga

SEQ ID NO: 197 - DNA, 78 bp, Thermoanaerobacterium saccharolyticum
  The mitochondrial localization signal sequence
atgttggcttctcgtcagctcctgggcctcgcccggagccgcgctgtcggcagcgcgagccttggtctccggcgcatg

SEQ ID NO: 198 - protein, 26 aa, Thermoanaerobacterium saccharolyticum
  amino acid sequence of the mitochondrial localization signal sequence
MLASRQLLGLARSRAVGSASLGLRRM

SEQ ID NO: 199 - DNA, 27 bp, Artificial sequence
  Primer
gaaggcgtgttggtgttgtagatgtcg

SEQ ID NO: 200 - DNA, 78 bp, Thermoanaerobacterium saccharolyticum
  The mitochondrial localization signal sequence
atgttcctgggcagccgcatccagacccgcgccttctcggcgtcggcccgaaacctctccaaggtcgccgttctcggt